(19) United States
(12) Patent Application Publication
(10) Pub. No.: US 2015/0293098 A1
(43) Pub. Date: Oct. 15, 2015

(54) METHODS FOR HEAD AND NECK CANCER PROGNOSIS

(71) Applicant: The University of North Carolina at Chapel Hill, Chapel Hill, NC (US)

(72) Inventors: David N. Hayes, Chapel Hill, NC (US); Matthew D. Wilkerson, Chapel Hill, NC (US); Vonn A. Walter, Chapel Hill, NC (US); Ni Zhao, Chapel Hill, NC (US)

(21) Appl. No.: 14/409,058

(22) PCT Filed: Jun. 17, 2013
(86) PCT No.: PCT/US2013/046136

§ 371 Date: Dec. 18, 2014

Related U.S. Application Data
(60) Provisional application No. 61/661,060, filed on Jun. 18, 2012.

(51) Int. Cl. G01N 33/574; C12Q 1/68

(52) U.S. Cl. CPC G01N 33/5743; C12Q 1/6886; G01N 2333/47; C12Q 2600/158; C12Q 2600/118; C12Q 2600/16

(57) ABSTRACT

This invention is directed to improved methods for determining the prognosis of patients with head and neck cancer. The invention is also directed to kits comprising reagents useful for determining head and neck cancer prognosis.

### CROSS REFERENCE TO RELATED APPLICATIONS

This application claims the benefit of 61/661,060 filed Jun. 18, 2012, Hayes et al., entitled “Method for Head and Neck Cancer Prognosis” having Atty. Docket No. UNC12004usv, which is hereby incorporated by reference in its entirety.

### STATEMENT REGARDING FEDERALLY SPONSORED RESEARCH OR DEVELOPMENT

This invention was made with government support under Grant No. K12-RR-023248 awarded by the National Institutes of Health. The government has certain rights in the invention.

### 1. FIELD OF THE INVENTION

This invention relates generally to the discovery of improved methods for determining the prognosis of patients with head and neck cancer. The invention is also directed to kits comprising reagents useful for determining head and neck cancer prognosis.

### 2. BACKGROUND OF THE INVENTION

2.1. HPV and Head and Neck Cancer

Head and neck squamous cell carcinoma (HNSCC) diagnoses constitute approximately 3-5 percent of all cancers with an estimate of 49,000 new cases and 11,000 deaths in 2010 in the US (Jemal et al., 2010; National Cancer Institute, 2005). Recent epidemiological data suggest an increasing incidence rate among younger people who are often non-smokers and non-drinkers (Curado & Hashibe, 2009; Marur et al., 2010; Patel et al., 2011; Schantz & Yu, 2002; Shiboski et al., 2005), which are frequently attributable to human papillomavirus (HPV) infection (Chaturvedi et al., 2011; Dahlstrand et al., 2004; El-Mofty & Lu, 2003; Franceschi et al., 1996; Furniss et al., 2007). HPV positive tumors are typically found in the oropharynx and have better response to treatment (Fakhry et al., 2008) and better disease outcome (Ang et al., 2010; Hafkamp et al., 2008). There is significant consensus that knowledge of patient HPV status will increasingly play a role in the management of this disease.

However, assessment of risk in the context of HPV infection has ongoing challenges. Perhaps chief among these is the fact that the diagnostic tests for the infection have limitations, and secondly, that smoking appears to degrade the favorable outcomes in patients with HPV-associated cancers for reasons that are unclear. There are two broad categories of assays for HPV. In the first category are tests for the virus itself including polymerase chain reaction, immunohistochemistry (IHC), and in situ hybridization. Alternatively, HPV status can be assessed indirectly through the p16 biomarker which is generally highly expressed in the setting of HPV infection. Detection of HPV directly suffers from a variety of limitations including both false positives and false negatives depending on the setting for reasons that have been extensively reviewed (Gillison et al., 2000; Ha et al., 2002; Shroyer & Greer, 1991; Stevens et al., 2011; Termine et al., 2008). Recently, large clinical trials have addressed the false positive concern primarily by assessing HPV only in the oropharynx, assuming that most positive tests outside the oropharynx would be false positives. The concern for false negatives has frequently been addressed with the addition of the biomarker p16 which is highly correlated with HPV infection because it is generally believed that HPV in situ hybridization is less sensitive and more specific than p16 staining (Begum et al., 2007; Schache et al., 2011; Stevens et al., 2011). In fact, recent studies have consistently shown favorable correlation between the two biomarkers, with nearly all HPV positive samples also staining for p16 (Begum et al., 2007). Interestingly, however, there is also a consistent pattern of p16 positive, HPV negative oropharynx tumors on the order of approximately 20% (Ang et al., 2010). Strikingly, p16 negative, HPV positive tumors, are rare, however. Most commonly, the p16 positive, HPV negative case has been attributed to a failed test of HPV, such as the presence of an HPV subtype not assessed by the assay. Such an explanation fails to address the fact that p16 is frequently positive in HNSCC outside the oropharynx, where HPV infection has generally been classified as a rare event. Interestingly, p16 positivity within the oropharynx appears to be at least as good a marker of favorable outcome, independent of whether samples also stained for HPV (Ang et al., 2010; Reimers et al., 2007). Yet outside the oropharynx, p16 has only infrequently been reported as a favorable marker (Harris et al., 2010b)

In addition to the complex story involving tumor site (oropharynx) and the biomarkers p16 and/or HPV is the fact that risk is also modified by smoking (Ang et al., 2010). Patients with greater smoking histories appear to have their favorable outcomes significantly tempered relative to nonsmoking HPV/p16 positive oropharynx cases for reasons that are not explained by the biomarker staining alone. Ang et al. documented at least 30% chance of death at 3 years for HPV positive patients with positive smoking histories (Ang et al., 2010). There is little question that HPV positive/p16 positive nonsmoking patients have more favorable outcomes. However, in patient populations with high or modest smoking rate, it is still valuable to assess patients' survival beyond HPV status.

2.2. Head and Neck Cancer Molecular Subtypes

Risk factors associated with HNSCC include smoking, alcohol use, rare germline cancer syndromes, and infection with the human papilloma virus (HPV). Although tumor site, TNM stage, and HPV status are useful in stratifying patient populations for prognosis and treatment (2), significant shortcomings remain in the characterization of patient outcomes based on these factors alone. For example, while it is widely recognized that HPV+ patients have better outcomes than HPV− patients, the favorable status is significantly attenuated by even modest smoking histories (3). Additionally, within patients who are HPV− and have at least 1 positive lymph node, overall disease mortality can approach 50% with few credible biologic risk factors separating those who do well from those who do not (4). The results of numerous recent studies suggest that molecular markers provide useful information that complements traditional prognostic data. Unfortunately the large number of putative markers and generally small sample sizes challenges the field to identify the most relevant patterns to pursue with primary focus.

Our group and others have suggested molecular subtypes of cancer as a means to prioritize the dominant genomic patterns within a specific tumor group (5-7). Validated subtypes based primarily on gene expression (GE) profiling of breast cancer, lymphoma, glioblastoma, lung cancer, and others have garnered broad interest (5-7). Preliminary work has suggested that such molecular groups are also found in head and neck cancer (8), but no confirmatory studies have been done. One issue limiting the investigation of HNSCC is the fact that cell lines evaluated in the context of the subtypes failed to convey ready models systems. Additionally, no data supporting underlying subtype-specific genomic alterations has yet emerged to suggest specific etiology of the patterns of gene expression. While there was the suggestion of a clinical benefit for one of the HNSCC subtypes, the cohort was small and the finding has not been repeated. In our opinion, for the HNSCC subtypes to move forward as a model for understanding this complex set of diseases the following progress is required. The subtypes should be shown to be statistically validated, genomic alterations underlying the subtypes should be documented, and at least preliminary model systems should be suggested.

Despite recent advances, the challenge of cancer treatment remains to target specific treatment regimens to pathogenically distinct tumor types, and ultimately personalize tumor treatment in order to maximize outcome. In particular, once a patient is diagnosed with cancer, such as head and neck cancer, there is a need for methods that allow the physician to predict the expected course of disease, including the likelihood of cancer recurrence, long-term survival of the patient and the like, and select the most appropriate treatment options accordingly. Such methods should specifically distinguish head and neck cancer patients with a poor prognosis from those with a good prognosis and permit the identification of high-risk, early-stage head and neck cancer patients who are likely to need aggressive therapy.

### 3. SUMMARY OF THE INVENTION

In particular non-limiting embodiments, the present invention provides a method for determining a prognosis for a patient with head and neck cancer which comprises: (a) obtaining a suitable patient sample; (b) measuring a nuclear p16 expression level; and (c) comparing the nuclear p16 expression level from the patient sample with an expression level for a control sample, wherein the nuclear p16 expression level is indicative of the prognosis for the patient with head and neck cancer.

In yet another embodiment, the invention provides a method for determining a prognosis for a patient with head and neck cancer which comprises: (a) obtaining a suitable patient sample; (b) measuring a level of CCND1; and (c) comparing the level of CCND1 from the patient sample with a level of CCND1 for a control sample, wherein the level of CCND1 is indicative of the prognosis for the patient with head and neck cancer.

In alternative embodiments, the invention provides a method for determining a prognosis for a patient with a solid tumor which comprises: (a) obtaining a suitable patient sample; (b) measuring p16 and RB1 genotypes, a CCND1 copy number, and a p16 nuclear protein expression level; and (c) comparing the p16 and RB1 genotypes, the CCND1 copy number, and the p16 nuclear protein expression level from the patient sample with p16 and RB1 genotypes, a CCND1 copy number, and a p16 nuclear protein expression level associated with a control sample, wherein the p16 and RB1 genotypes, the CCND1 copy number, and the p16 nuclear protein expression level are indicative of the prognosis for the patient with the solid tumor.

The invention also provides method for determining an appropriate radiation and/or chemotherapy protocol, the likelihood of cancer recurrence, monitoring the progress of a treatment protocol for a patient with head and neck cancer which comprises: (a) obtaining a suitable patient sample; (b) measuring a nuclear p16 expression level; and (c) comparing the nuclear p16 expression level from the patient sample with a level associated with a control sample, wherein the nuclear p16 expression level is indicative of the appropriate radiation and/or chemotherapy protocol, the likelihood of cancer recurrence, or monitoring the progress of a treatment protocol.

Kits to practice the methods described herein are also provided.

### 4. BRIEF DESCRIPTION OF THE FIGURES

FIG. 1: FIG. 1A (Panel A) shows the CDKN2A locus and the p16INK4a alteration rate. FIG. 1B (Panel B) shows the relationship between the forms of p16INK4a (mutated, methylated, RB1 altered or fusion). FIG. 1C (Panel C) shows the fusion between KIAA1797 and p16INK4a. FIG. 1D (Panel D) shows alterations in p16INK4a, RB1, CDK6 and CCND1.

FIG. 2: Representative examples of p16 immunostaining in head and neck squamous cell carcinoma. Immunohistochemical staining for p16 expression of head and neck squamous cell carcinoma was evaluated by product scores in different cellular compartments separately. From the above left: (Panel A) p16 high expression in both nuclei and cytoplasm; (Panel B) p16 low expression in both nuclei and cytoplasm; (Panel C) High nuclear expression and modest cytoplasmic staining (however, by our scoring this still qualified at the lowest end of “high cytoplasmic”); (Panel D) High cytoplasmic expression and low nuclear expression.

FIG. 3: Distributions of p16 staining product scores

FIGS. 4A and 4B: Kaplan Meier estimates of overall survival (FIG. 4A) and progression free survival (FIG. 4B) according to p16 expression in whole study population. All survival estimates were censored at 60 months. Abbreviations: HN, high nuclear, any cytoplasmic staining; HC, high cytoplasmic, low nuclear staining; LS, low nuclear, low cytoplasmic staining

FIG. 5A-5D: Gene Expression Subtypes in Head and Neck Squamous Cell Carcinoma. Heatmaps of the expression values of the 840 classifier genes: FIG. 5A (A) and select genes associated with HNSCC FIG. 5B (B) for each of the expression subtypes. Validation heatmaps of the centroid-based distances between the centroids of the expression subtypes in the current study and those from Chung et al. FIG. 5C (C) and Wilkerson et al. FIG. 5D (D).

FIG. 6A-6B: Copy Number Gains and Losses in the Expression Subtypes. Plots of the mean copy number values in the HNSCC expression subtypes after smoothing and outlier removal, both genome-wide (FIG. 6A) and for specific chromosomes of interest (FIG. 6B).

FIG. 7A-7B: Average Gene Expression and Copy Number by Expression Subtype. Mean gene-specific copy number (CN) and gene expression (GE) values in the HNSCC expression subtypes for genes in the chr3q amplicon (FIG. 7A) and elsewhere in the genome (FIG. 7B).

FIG. 8A-8D: Recurrence-Free Survival in Expression Subtypes. Kaplan-Meier plots and Log-Rank Test p-values comparing recurrence-free survival times in all expression subtypes (FIG. 8A), HPV+ vs. HPV− subjects (FIG. 8B), all expression subtypes in HPV-subjects (FIG. 8C), and AT vs. non-AT in HPV− subjects (FIG. 8D).

FIG. 9A-9D: Evidence Supporting the Presence of Four Expression Subtypes. (FIG. 9A) Heatmap of the ConsensusClusterPlus dissimilarity matrix for the 138 subjects and 2500 most variable genes (k=4). (FIG. 9B) ConsensusClusterPlus tracking plot for the 138 subjects and 2500 most variable genes. (FIG. 9C) Silhouette plots for 138 subjects and the 840 classifier genes. (FIG. 9D) SigClust p-values for all pairwise comparisons of the expression subtypes.

FIG. 10: Kaplan-Meier Curves for CCND1 Copy Number Gains. Kaplan-Meier curves illustrating recurrence-free survival times for subjects with and without CCND1 copy number gains.

FIG. 11: Kaplan-Meier Curves Illustrating Two Groups with Poor Survival Outcomes. Kaplan-Meier curves illustrating recurrence-free survival times for four mutually exclusive groups of patients: (1) HPV+ subjects (HPV+), (2) HPV− patients with CCND1 gains (CCND1 Gain), (3) HPV− patients without CCND1 gains that are AT (HPV− AT), (4) all remaining patients (Other).

FIG. 12: Genome-Wide Mean Copy Number Values in HNSCC Cell Lines. Genome-wide plot of the mean copy number values for each of the predicted subtypes based on the HNSCC samples in the Cancer Cell Line Encyclopedia data.

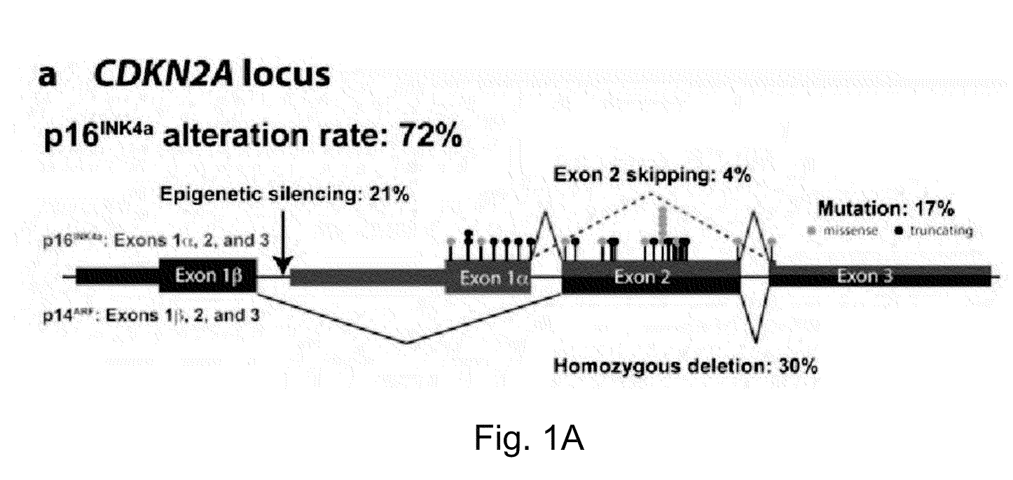

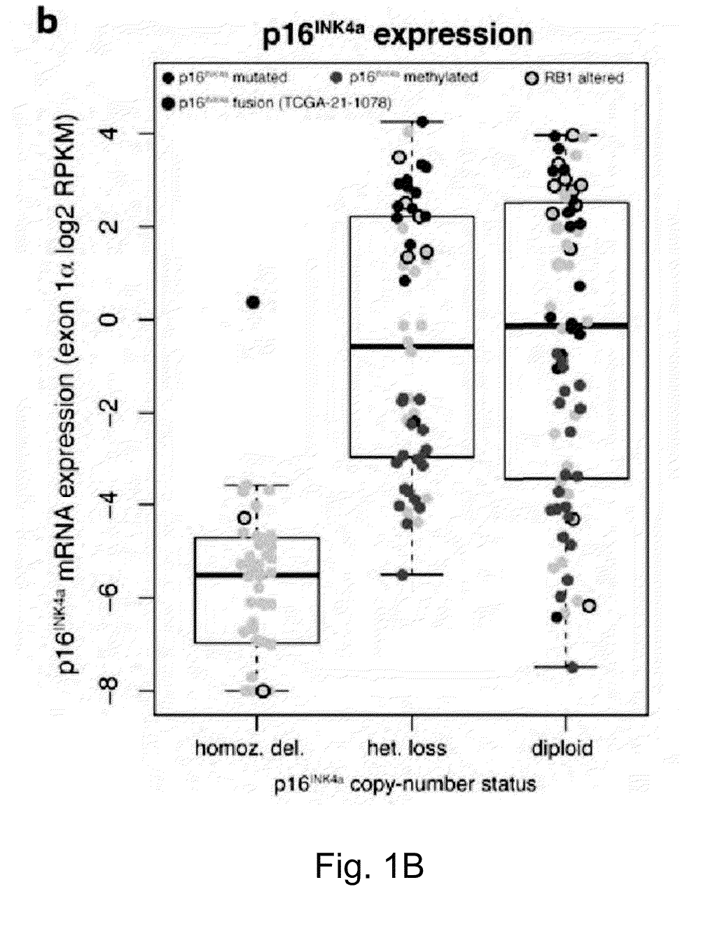

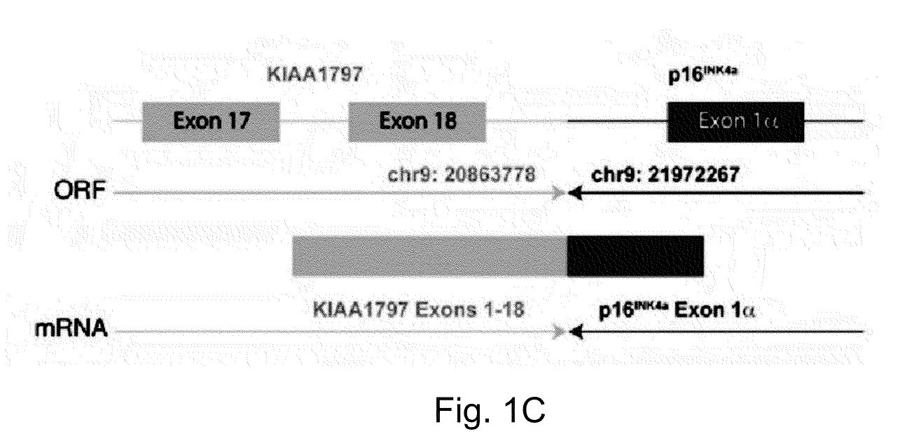

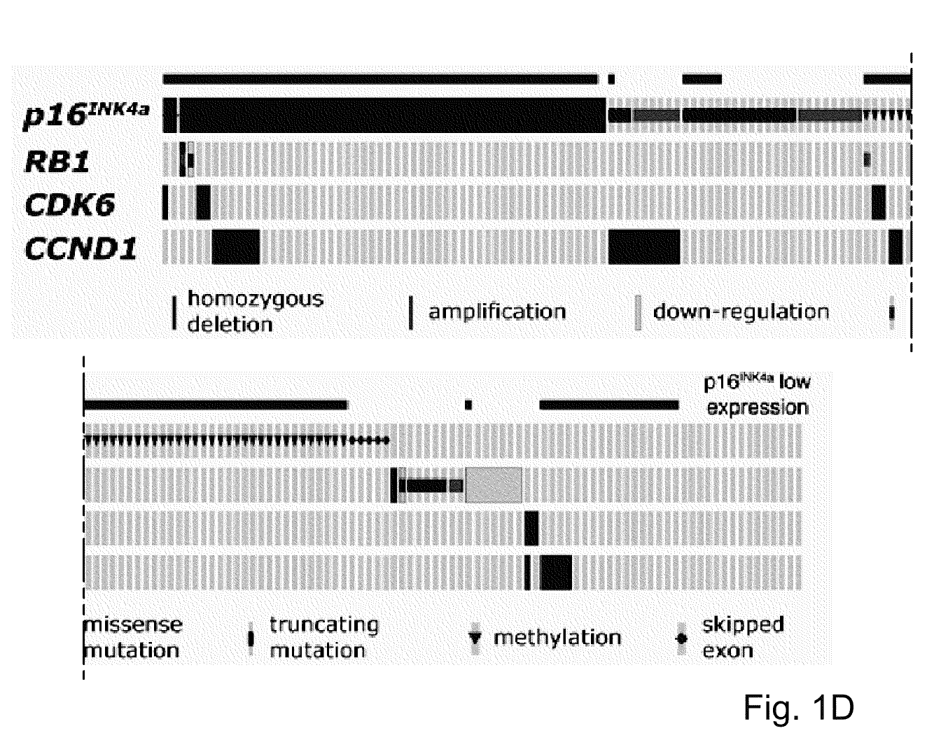

### 5. DETAILED DESCRIPTION OF THE INVENTION

This invention In particular non-limiting embodiments, the present invention provides a method for determining a prognosis for a patient with head and neck cancer which comprises: (a) obtaining a suitable patient sample; (b) measuring a nuclear p16 expression level; and (c) comparing the nuclear p16 expression level from the patient sample with an expression level for a control sample, wherein the nuclear p16 expression level is indicative of the prognosis for the patient with head and neck cancer.

In yet another embodiment, the invention provides a method for determining a prognosis for a patient with head and neck cancer which comprises: (a) obtaining a suitable patient sample; (b) measuring a level of CCND1; and (c) comparing the level of CCND1 from the patient sample with a level of CCND1 for a control sample, wherein the level of CCND1 is indicative of the prognosis for the patient with head and neck cancer.

In alternative embodiments, the invention provides a method for determining a prognosis for a patient with a solid tumor which comprises: (a) obtaining a suitable patient sample; (b) measuring p16 and RB1 genotypes, a CCND1 copy number, and a p16 nuclear protein expression level; and (c) comparing the p16 and RB1 genotypes, the CCND1 copy number, and the p16 nuclear protein expression level from the patient sample with p16 and RB1 genotypes, a CCND1 copy number, and a p16 nuclear protein expression level associated with a control sample, wherein the p16 and RB1 genotypes, the CCND1 copy number, and the p16 nuclear protein expression level are indicative of the prognosis for the patient with the solid tumor.

This embodiment of the invention may further comprise measuring the expression of genes associated with an atypical subtype. The solid tumor may be a solid tumor of epithelial origin, a squamous cell carcinoma or a melanoma.

The invention also provides method for determining an appropriate radiation and/or chemotherapy protocol, the likelihood of cancer recurrence, monitoring the progress of a treatment protocol for a patient with head and neck cancer which comprises: (a) obtaining a suitable patient sample; (b) measuring a nuclear p16 expression level; and (c) comparing the nuclear p16 expression level from the patient sample with a level associated with a control sample, wherein the nuclear p16 expression level is indicative of the appropriate radiation and/or chemotherapy protocol, the likelihood of cancer recurrence, or monitoring the progress of a treatment protocol.

In these methods, the nuclear p16 expression level may be reduced and the reduction is due to mutations or copy number loss. The mutations may be acquired (or somatic) mutations or hereditary mutations. The expression may be reduced due to methylation. The method may further comprise measuring levels of RB1 and p53 and a reduced level of RB1 or p53 in combination with a reduced nuclear p16 expression level indicates a poor prognosis. Alternatively, the method may further comprise measuring levels of CCND1 or levels of expression associated with the atypical subtype wherein increased levels of CCND1 or levels of expression associated with the atypical subtype are indicative of a poor prognosis. The method may also further comprises measuring a cytoplasmic p16 expression level, wherein if the nuclear p16 expression level is reduced and the cytoplasmic p16 level is elevated in indicative of a particularly poor prognosis.

The invention also includes methods of selecting patients for treatment by both radiation and chemotherapy. In particular, low nuclear p16 expression levels indicate a poor prognosis thus a patient that previously would have received just radiation as the standard care should receive both radiation and chemotherapy. Alternatively, elevated nuclear p16 expression levels indicate a good prognosis thus a patient that previously would have received both radiation and chemotherapy as the standard care, should receive only radiation.

The expression levels may be measured by an mRNA assay or a protein assay such as antibodies. The patient sample may be a biopsy sample, a FFPE sample or a lymph node biopsy sample. The head and neck cancer may be a squamous cell carcinoma (SCC). The head and neck cancer may be a hypopharynx, a glottis larynx, a larynx, a lip, a nasopharynx, an oral cavity, a salivary gland, a sinus, or a superglottic larynx cancer.

The invention also includes methods of identifying patients for particular treatments or selecting patients for which a particular treatment would be desirable or contraindicated.

The methods above may be performed by a reference laboratory, a hospital pathology laboratory or a doctor. The methods above may further comprise an algorithm. For example an algorithm to analyze the nuclear p16, RB1 and p53 expression levels or an algorithm to analyze expression levels associated with particular subtypes of head and neck cancer.

Kits to practice the methods described herein are also provided.

Unlike methods previously described, the methods described herein may be widely used in all types of head and neck cancer. These methods are independent of smoking status or HPV status.

P16 Invention

Background: Recently the management of head and neck squamous cell carcinoma (HNSCC) has focused considerable attention on biomarkers, which may influence outcomes. Tests for human papilloma infection, including direct assessment of the virus as well as an associated tumor suppressor gene p16, are considered reproducible. Tumors from familial melanoma syndromes, have suggested that nuclear localization of p16 might play a further role in risk stratification. We hypothesized p16 staining that considered nuclear localization might be informative for predicting outcomes in a broader set of HNSCC tumors not limited to the oropharynx, HPV status or by smoking status.

Methods: Patients treated for HNSCC from 2002 to 2006 at UNC hospitals that had banked tissue available were eligible for this study. Tissue microarrays (TMA) were generated in triplicate Immunohistochemical (IHC) staining for p16 was performed and scored separately for nuclear and cytoplasmic staining. Human papilloma virus (HPV) staining was also carried out using monoclonal antibody E6H4. p16 expression, HPV status and other clinical features were correlated with progression-free (PFS) and overall survival (OS).

Results: 135 patients had sufficient sample for this analysis. Median age at diagnosis was 57 years (range 20-82), with 68.9% males, 8.9% never smokers and 32.6% never drinkers. Three year OS rate and PFS rate was 63.0% and 54.1%, respectively. Based on the p16 staining score, patients were divided into three groups: high nuclear, any cytoplasmic staining group (HN), low nuclear, low cytoplasmic staining group (LS) and high cytoplasmic, low nuclear staining group (HC). The HN and the LS groups had significantly better overall survival than the HC group with hazard ratios of 0.1 and 0.37, respectively, after controlling for other factors, including HPV status. These two groups also had significantly better progression-free survival than the HC staining group. This finding was consistent for sites outside the oropharynx, and did not require adjustment for smoking status.

Conclusions: Different p16 protein localization suggested different survival outcomes in a manner that does not require limiting the biomarker to the oropharynx and does not require assessment of smoking status. A biomarker that more precisely captures the biology of both smoking and tumor site, and that unifies the frequent discrepancies between HPV staining and p16 staining would be welcome.

Recently our group reported that p16 staining was prognostic in a set of young patients with HNSCC who were confirmed HPV negative by PCR and in situ hybridization, (Harris et al., 2010b), leading us to question whether p16 alone could be extended to evaluate risk outside the oropharynx.

Smoking and HPV infection are two important etiologies of p16 alteration in HNSCC. In HPV infected patients, the protein RB1 is inactivated by viral oncoprotein E7, leading to a high and nuclear localized p16 expression (Andl et al., 1998; Li et al., 2004; Marur et al., 2010; Wiest et al., 2002). In contrast, in situations where p16 is retained but altered in function by mutation or other genetic events, we may still observe modest to high p16 expression, but with abnormal cellular localization. In many additional smoking patients, p16 can be lost via more deleterious genetic or epigenetic changes, such as homozygous deletion, nonsense mutation, or perhaps methylation and gene silencing. On the basis of these etiologic differences, we expected to observe distinct patterns in p16 IHC staining. Similar hypotheses of p16's role in prognosis have been tested in other tumor types. For example, in high-grade astrocytoma, a study has shown that nucleus-located p16 is associated with better disease outcome while cytoplasmic p16 indicates worse patients' survival (Arifin et al., 2006). In other tumor types, including endometrial cancers, melanoma, and astrocytomas (Arifin et al., 2006; Emig et al., 1998; Ghiorzo et al., 2004; Milde-Langosch et al., 2001; Salvesen et al., 2000; Straume et al., 2000), reports also exist where p16 localization is associated with disease outcomes. As p16 protein acts as a cell cycle inhibitor in the nucleus, we proposed that nuclear p16 staining and cytoplasmic p16 staining may have a distinct prognostic effect in HNSCC. We tested this hypothesis in a population-based patient cohort—the Carolina Head and Neck Cancer Study (CHANCE).

HNSCC Subtypes

Head and neck squamous cell carcinoma (HNSCC) is a heterogeneous disease whose underlying etiology has not been explained by traditional prognostic factors such as tumor site, TNM stage, and HPV status. Although previous studies have detected molecular subtypes of HNSCC, these subtypes have not been validated in independent datasets or detected in cell lines, nor has the benefit of such a classification scheme been fully realized. We show that molecular subtypes of HNSCC exist; that these subtypes have distinct patterns of chromosomal gain and loss, some of which affect canonical oncogenes and tumor suppressors; and that the subtypes are biologically and clinically relevant. In addition, we validate our findings in independent tumor, cell line, and tissue microarray datasets. These subtypes provide new insight into HNSCC etiology, as well as a valuable method for classifying HNSCC tumors.

The biomarkers of the invention include genes and proteins. Such biomarkers include DNA comprising the entire or partial sequence of the nucleic acid sequence encoding the biomarker, or the complement of such a sequence. The biomarker nucleic acids also include RNA comprising the entire or partial sequence of any of the nucleic acid sequences of interest. A biomarker protein is a protein encoded by or corresponding to a DNA biomarker of the invention. A biomarker protein comprises the entire or partial amino acid sequence of any of the biomarker proteins or polypeptides. Fragments and variants of biomarker genes and proteins are also encompassed by the present invention. By “fragment” is intended a portion of the polynucleotide or a portion of the amino acid sequence and hence protein encoded thereby. Polynucleotides that are fragments of a biomarker nucleotide sequence generally comprise at least 10, 15, 20, 50, 75, 100, 150, 200, 250, 300, 350, 400, 450, 500, 550, 600, 650, 700, 800, 900, 1,000, 1,200, or 1,500 contiguous nucleotides, or up to the number of nucleotides present in a full-length biomarker polynucleotide disclosed herein. A fragment of a biomarker polynucleotide will generally encode at least 15, 25, 30, 50, 100, 150, 200, or 250 contiguous amino acids, or up to the total number of amino acids present in a full-length biomarker protein of the invention. “Variant” is intended to mean substantially similar sequences. Generally, variants of a particular biomarker of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that biomarker as determined by sequence alignment programs.

The biomarkers of the invention include genes and proteins. Such biomarkers include DNA comprising the entire or partial sequence of the nucleic acid sequence encoding the biomarker, or the complement of such a sequence. The biomarker nucleic acids also include RNA comprising the entire or partial sequence of any of the nucleic acid sequences of interest. A biomarker protein is a protein encoded by or corresponding to a DNA biomarker of the invention. A biomarker protein comprises the entire or partial amino acid sequence of any of the biomarker proteins or polypeptides. Fragments and variants of biomarker genes and proteins are also encompassed by the present invention. By “fragment” is intended a portion of the polynucleotide or a portion of the amino acid sequence and hence protein encoded thereby. Polynucleotides that are fragments of a biomarker nucleotide sequence generally comprise at least 10, 15, 20, 50, 75, 100, 150, 200, 250, 300, 350, 400, 450, 500, 550, 600, 650, 700, 800, 900, 1,000, 1,200, or 1,500 contiguous nucleotides, or up to the number of nucleotides present in a full-length biomarker polynucleotide disclosed herein. A fragment of a biomarker polynucleotide will generally encode at least 15, 25, 30, 50, 100, 150, 200, or 250 contiguous amino acids, or up to the total number of amino acids present in a full-length biomarker protein of the invention. “Variant” is intended to mean substantially similar sequences. Generally, variants of a particular biomarker of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that biomarker as determined by sequence alignment programs.

A “biomarker” is a gene or protein whose level of expression in a tissue or cell is altered compared to that of a normal or healthy cell or tissue. The biomarkers of the present invention are genes and proteins whose overexpression correlates with cancer, particularly head and neck cancer, prognosis. As used herein, “overexpression” means expression greater than the expression detected in normal, non-cancerous tissue. For example, an RNA transcript or its expression product that is overexpressed in a cancer cell or tissue may be expressed at a level that is 1.5 times higher than in a in normal, non-cancerous cell or tissue, such as 2 times higher, 3 times higher, 5 times higher, or more times higher.

In some embodiments, overexpression, such as of an RNA transcript or its expression product, is determined by normalization to the level of reference RNA transcripts or their expression products, which can be all measured transcripts (or their products) in the sample or a particular reference set of RNA transcripts (or their products). Normalization is performed to correct for or normalize away both differences in the amount of RNA assayed and variability in the quality of the RNA used. Therefore, an assay typically measures and incorporates the expression of certain normalizing genes, including well known housekeeping genes, such as, for example, GAPDH and/or β-Actin. Alternatively, normalization can be based on the mean or median signal of all of the assayed biomarkers or a large subset thereof (global normalization approach).

In particular embodiments, selective overexpression of a biomarker or combination of biomarkers of interest in a patient sample is indicative of a poor cancer prognosis. By “indicative of a poor prognosis” is intended that overexpression of the particular biomarker or combination of biomarkers is associated with an increased likelihood of relapse or recurrence of the underlying cancer or tumor, metastasis or death. For example, “indicative of a poor prognosis” may refer to an increased likelihood of relapse or recurrence of the underlying cancer or tumor, metastasis, or death within ten years, such as five years. In other aspects of the invention, the absence of overexpression of a biomarker or combination of biomarkers of interest is indicative of a good prognosis. As used herein, “indicative of a good prognosis” refers to an increased likelihood that the patient will remain cancer-free. In some embodiments, “indicative of a good prognosis” refers to an increased likelihood that the patient will remain cancer-free for ten years, such as five years.

5.1. Samples

In particular embodiments, the methods for evaluating head and neck cancer prognosis include collecting a patient body sample having a cancer cell or tissue, such as a head and neck tissue sample or a primary head and neck tumor tissue sample. The head and neck sample may be from the larynx with following three anatomical regions: (i) supraglottic larynx includes the epiglottis, false vocal cords, ventricles, aryepiglottic folds, and arytenoids; (ii) glottis includes the true vocal cords and the anterior and posterior commissures; and the subglottic region begins about 1 cm below the true vocal cords and extends to the lower border of the cricoid cartilage or the first tracheal ring. The sample may be from the lip or the oral cavity, e.g., buccal mucosa, lower gingiva, upper gingiva, hard palate, lip, floor of mouth, retromolar trigone, or anterior two thirds of tongue. The sample may be from the oropharynx, e.g., the base of the tongue including the pharyngoepiglottic folds and the glossoepiglottic folds; the tonsillar region including the fossa and the anterior and posterior pillars; the soft palate, including the uvula; or the pharyngeal walls.

By “body sample” is intended any sampling of cells, tissues, or bodily fluids in which expression of a biomarker can be detected. Examples of such body samples include, but are not limited to, biopsies and smears. Bodily fluids useful in the present invention include blood, lymph, urine, saliva, nipple aspirates, gynecological fluids, or any other bodily secretion or derivative thereof. Blood can include whole blood, plasma, serum, or any derivative of blood. In some embodiments, the body sample includes head and neck cells, particularly head and neck tissue from a biopsy, such as a head and neck tumor tissue sample. Body samples may be obtained from a patient by a variety of techniques including, for example, by scraping or swabbing an area, by using a needle to aspirate cells or bodily fluids, or by removing a tissue sample (i.e., biopsy). Methods for collecting various body samples are well known in the art. In some embodiments, a head and neck tissue sample is obtained by, for example, fine needle aspiration biopsy, core needle biopsy, or excisional biopsy. Fixative and staining solutions may be applied to the cells or tissues for preserving the specimen and for facilitating examination. Body samples, particularly head and neck tissue samples, may be transferred to a glass slide for viewing under magnification. In one embodiment, the body sample is a formalin-fixed, paraffin-embedded (FFPE) head and neck tissue sample, particularly a primary head and neck tumor sample.

5.2. Compositions and Kits

The invention provides compositions and kits for determining the prognosis of a patient with head and neck cancer which comprises: (a) a means for measuring a nuclear p16 expression level; and (b) instructions for comparing the nuclear p16 expression level from patient sample with a nuclear p16 expression level for a patient control, wherein a reduced nuclear p16 expression level is indicative a poor prognosis for the patient with head and neck cancer.

Alternatively, the invention provides a kit comprising: a reagent selected from a group consisting of: (a) nucleic acid probes capable of specifically hybridizing with nucleic acids from p16; (b) a pair of nucleic acid primers capable of PCR amplification of p16; (c) antibodies specific for p16; and (d) instructions for use in measuring nuclear p16 expression levels in a tissue sample from a patient with head and neck cancer.

Any methods available in the art for detecting expression of biomarkers are encompassed herein. The expression of a biomarker of the invention can be detected on a nucleic acid level (e.g., as an RNA transcript) or a protein level. By “detecting expression” is intended determining the quantity or presence of an RNA transcript or its expression product of a biomarker gene. Thus, “detecting expression” encompasses instances where a biomarker is determined not to be expressed, not to be detectably expressed, expressed at a low level, expressed at a normal level, or overexpressed. In order to determine overexpression, the body sample to be examined can be compared with a corresponding body sample that originates from a healthy person. That is, the “normal” level of expression is the level of expression of the biomarker in, for example, a head and neck tissue sample from a human subject or patient not afflicted with head and neck cancer. Such a sample can be present in standardized form. In some embodiments, determination of biomarker overexpression requires no comparison between the body sample and a corresponding body sample that originates from a healthy person. For example, detection of overexpression of a biomarker indicative of a poor prognosis in a head and neck tumor sample may preclude the need for comparison to a corresponding head and neck tissue sample that originates from a healthy person. Moreover, in some aspects of the invention, no expression, underexpression, or normal expression (i.e., the absence of overexpression) of a biomarker or combination of biomarkers of interest provides useful information regarding the prognosis of a head and neck cancer patient.

Methods for detecting expression of the biomarkers of the invention, that is, gene expression profiling, include methods based on hybridization analysis of polynucleotides, methods based on sequencing of polynucleotides, immunohistochemistry methods, and proteomics-based methods. The most commonly used methods known in the art for the quantification of mRNA expression in a sample include northern blotting and in situ hybridization (Parker and Barnes, Methods Mol. Biol. 106:247-83, 1999), RNAse protection assays (Hod, Biotechniques 13:852-54, 1992), PCR-based methods, such as reverse transcription PCR(RT-PCR) (Weis et al., TIG 8:263-64, 1992), and array-based methods (Schena et al., Science 270:467-70, 1995). Alternatively, antibodies may be employed that can recognize specific duplexes, including DNA duplexes, RNA duplexes, and DNA-RNA hybrid duplexes, or DNA-protein duplexes. Representative methods for sequencing-based gene expression analysis include Serial Analysis of Gene Expression (SAGE) and gene expression analysis by massively parallel signature sequencing.

The term “probe” refers to any molecule that is capable of selectively binding to a specifically intended target biomolecule, for example, a nucleotide transcript or a protein encoded by or corresponding to a biomarker. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Hybridization Analysis of Polynucleotides

In some embodiments, the expression of a biomarker of interest is detected at the nucleic acid level. Nucleic acid-based techniques for assessing expression are well known in the art and include, for example, determining the level of biomarker RNA transcripts (i.e., mRNA) in a body sample. Many expression detection methods use isolated RNA. The starting material is typically total RNA isolated from a body sample, such as a tumor or tumor cell line, and corresponding normal tissue or cell line, respectively. Thus RNA can be isolated from a variety of primary tumors, including breast, lung, colon, prostate, brain, liver, kidney, pancreas, spleen, thymus, testis, ovary, uterus, and the like, or tumor cell lines. If the source of mRNA is a primary tumor, mRNA can be extracted, for example, from frozen or archived paraffin-embedded and fixed (e.g., formalin-fixed) tissue samples.

General methods for mRNA extraction are well known in the art and are disclosed in standard textbooks of molecular biology, including Ausubel et al., ed., Current Protocols in Molecular Biology, John Wiley & Sons, New York 1987-1999. Methods for RNA extraction from paraffin embedded tissues are disclosed, for example, in Rupp and Locker (Lab Invest. 56:A67, 1987) and De Andres et al. (Biotechniques 18:42-44, 1995). In particular, RNA isolation can be performed using a purification kit, a buffer set and protease from commercial manufacturers, such as Qiagen (Valencia, Calif.), according to the manufacturer's instructions. For example, total RNA from cells in culture can be isolated using Qiagen RNeasy mini-columns. Other commercially available RNA isolation kits include MasterPure™ Complete DNA and RNA Purification Kit (Epicentre, Madison, Wis.) and Paraffin Block RNA Isolation Kit (Ambion, Austin, Tex.). Total RNA from tissue samples can be isolated, for example, using RNA Stat-60 (Tel-Test, Friendswood, Tex.). RNA prepared from a tumor can be isolated, for example, by cesium chloride density gradient centrifugation. Additionally, large numbers of tissue samples can readily be processed using techniques well known to those of skill in the art, such as, for example, the single-step RNA isolation process of Chomczynski (U.S. Pat. No. 4,843,155).

Isolated mRNA can be used in hybridization or amplification assays that include, but are not limited to, Southern or Northern analyses, PCR analyses and probe arrays. One method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250, or 500 nucleotides in length and sufficient to specifically hybridize under stringent conditions to an mRNA or genomic DNA encoding a biomarker of the present invention. Hybridization of an mRNA with the probe indicates that the biomarker in question is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probes are immobilized on a solid surface and the mRNA is contacted with the probes, for example, in an Agilent gene chip array. A skilled artisan can readily adapt known mRNA detection methods for use in detecting the level of mRNA encoded by the biomarkers of the present invention.

An alternative method for determining the level of biomarker mRNA in a sample involves the process of nucleic acid amplification, for example, by RT-PCR (U.S. Pat. No. 4,683,202), ligase chain reaction (Barany, Proc. Natl. Acad. Sci. USA 88:189-93, 1991), self-sustained sequence replication (Guatelli et al., Proc. Natl. Acad. Sci. USA 87:1874-78, 1990), transcriptional amplification system (Kwoh et al., Proc. Natl. Acad. Sci. USA 86:1173-77, 1989), Q-Beta Replicase (Lizardi et al., Bio/Technology 6:1197, 1988), rolling circle replication (U.S. Pat. No. 5,854,033), or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, biomarker expression is assessed by quantitative fluorogenic RT-PCR (i.e., the TaqMan® System). For PCR analysis, well known methods are available in the art for the determination of primer sequences for use in the analysis.

Biomarker expression levels of RNA may be monitored using a membrane blot (such as used in hybridization analysis such as Northern, Southern, dot, and the like), or microwells, sample tubes, gels, beads, or fibers (or any solid support comprising bound nucleic acids). See, for example, U.S. Pat. Nos. 5,770,722, 5,874,219, 5,744,305, 5,677,195 and 5,445,934. The detection of biomarker expression may also comprise using nucleic acid probes in solution.

In one embodiment of the invention, microarrays are used to detect biomarker expression. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning Hybridization intensities for each probe on the array are determined and converted to a quantitative value representing relative gene expression levels. See, for example, U.S. Pat. Nos. 6,040,138, 5,800,992 and 6,020,135, 6,033,860, and 6,344,316. High-density oligonucleotide arrays are particularly useful for determining the gene expression profile for a large number of RNAs in a sample.

Techniques for the synthesis of these arrays using mechanical synthesis methods are described in, for example, U.S. Pat. No. 5,384,261. Although a planar array surface is generally used, the array can be fabricated on a surface of virtually any shape or even a multiplicity of surfaces. Arrays can be nucleic acids (or peptides) on beads, gels, polymeric surfaces, fibers (such as fiber optics), glass, or any other appropriate substrate. See, for example, U.S. Pat. Nos. 5,770,358, 5,789,162, 5,708,153, 6,040,193 and 5,800,992. Arrays can be packaged in such a manner as to allow for diagnostics or other manipulation of an all-inclusive device. See, for example, U.S. Pat. Nos. 5,856,174 and 5,922,591.

In a specific embodiment of the microarray technique, PCR amplified inserts of cDNA clones are applied to a substrate in a dense array. For example, at least 10,000 nucleotide sequences are applied to the substrate. The microarrayed genes, immobilized on the microchip at 10,000 elements each, are suitable for hybridization under stringent conditions. Fluorescently labeled cDNA probes can be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance.

With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93:106-49, 1996). Microarray analysis can be performed by commercially available equipment, following manufacturer's protocols, such as by using the Affymetrix GenChip technology, or Agilent ink jet microarray technology. The development of microarray methods for large-scale analysis of gene expression makes it possible to search systematically for molecular markers of cancer classification and outcome prediction in a variety of tumor types.

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. See, Velculescu et al. (Science 270:484-87, 1995; Cell 88:243-51, 1997).

An additional method of biomarker expression analysis at the nucleic acid level is gene expression analysis by massively parallel signature sequencing (MPSS), as described by Brenner et al. (Nat. Biotech. 18:630-34, 2000). This is a sequencing approach that combines non-gel-based signature sequencing with in vitro cloning of millions of templates on separate 5 μM diameter microbeads. First, a microbead library of DNA templates is constructed by in vitro cloning. This is followed by the assembly of a planar array of the template-containing microbeads in a flow cell at a high density (typically greater than 3.0×10⁶ microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

Epigenetic Modifications

The methods of the present invention may also be accompanied by and/or supplemented by methods for detecting post-translational modifications or epigenetic changes such as acetylation, methylation, phosphorylation, sumoylation, or ubiquitylation. Such epigenetic changes may occur on proteins, such as histone acetylation, kinase phosphorylation, or nucleic acids such as the 5′ methyl cytosine or 5′hydromethyl cytosine formation at CpG sites.

Methods for measuring epigenetic changes are known in the art, e.g., for nucleic acids: EP 1488008 B1 (Berlin), U.S. Pat. No. 7,960,112 (Budiman et al.), U.S. Pat. No. 7,666,589 (Levenson & Gartenhaus); U.S. Pat. No. 7,611,869 (Fan), U.S. Pat. No. 7,364,855 (Anderson et al.); PCT Pub. Nos. WO 2010/086389 (Weinhausel et al.); WO 2005/071106 (Berlin); WO 2005/033332 (Distler); WO 2003/023065 (Wang et al.); WO 1997/046705 (Herman & Baylin); for proteins U.S. Pat. No. 7,074,578 (Kouzarides and Santos-Rosa).

Immunohistochemistry

Immunohistochemistry methods are also suitable for detecting the expression levels of the biomarkers of the present invention. In one embodiment, a patient head and neck tissue sample is collected by, for example, biopsy techniques known in the art. Samples can be frozen for later preparation or immediately placed in a fixative solution. Tissue samples can be fixed by treatment with a reagent, such as formalin, gluteraldehyde, methanol, or the like and embedded in paraffin. Methods for preparing slides for immunohistochemical analysis from formalin-fixed, paraffin-embedded tissue samples are well known in the art.

In some instances, samples may need to be modified in order to make the biomarker antigens accessible to antibody binding. For example, formalin fixation of tissue samples results in extensive cross-linking of proteins that can lead to the masking or destruction of antigen sites and, subsequently, poor antibody staining As used herein, “antigen retrieval” or “antigen unmasking” refers to methods for increasing antigen accessibility or recovering antigenicity in, for example, formalin-fixed, paraffin-embedded tissue samples. Any method for making antigens more accessible for antibody binding may be used in the practice of the invention, including those antigen retrieval methods known in the art. See, for example, Hanausek and Walaszek, eds. (1998) Tumor Marker Protocols (Humana Press, Inc., Totowa, N.J.) and Shi et al., eds. (2000) Antigen Retrieval Techniques: Immunohistochemistry and Molecular Morphology (Eaton Publishing, Natick, Mass.).

Antigen retrieval methods include but are not limited to treatment with proteolytic enzymes (e.g., trypsin, chymotrypsin, pepsin, pronase, and the like) or antigen retrieval solutions. Antigen retrieval solutions of interest include, for example, citrate buffer, pH 6.0, Tris buffer, pH 9.5, EDTA, pH 8.0, L.A.B. (“Liberate Antibody Binding Solution,” Polysciences, Warrington, Pa.), antigen retrieval Glyca solution (Biogenex, San Ramon, Calif.), citrate buffer solution, pH 4.0, Dawn® detergent (Proctor & Gamble, Cincinnati, Ohio), deionized water, and 2% glacial acetic acid. In some embodiments, antigen retrieval comprises applying the antigen retrieval solution to a formalin-fixed tissue sample and then heating the sample in an oven (e.g., at 60° C.), steamer (e.g., at 95° C.), or pressure cooker (e.g., at 120° C.) at specified temperatures for defined time periods. In other aspects of the invention, antigen retrieval may be performed at room temperature. Incubation times will vary with the particular antigen retrieval solution selected and with the incubation temperature. For example, an antigen retrieval solution may be applied to a sample for as little as 5, 10, 20, or 30 minutes or up to overnight. The design of assays to determine the appropriate antigen retrieval solution and optimal incubation times and temperatures is standard and well within the routine capabilities of those of ordinary skill in the art.

Following antigen retrieval, samples are blocked using an appropriate blocking agent (e.g., hydrogen peroxide). An antibody directed to a biomarker of interest is then incubated with the sample for a time sufficient to permit antigen-antibody binding. In particular embodiments, at least five antibodies directed to five distinct biomarkers are used to evaluate the prognosis of a head and neck cancer patient. Where more than one antibody is used, these antibodies may be added to a single sample sequentially as individual antibody reagents, or simultaneously as an antibody cocktail. Alternatively, each individual antibody may be added to a separate tissue section from a single patient sample, and the resulting data pooled.

Techniques for detecting antibody binding are well known in the art. Antibody binding to a biomarker of interest can be detected through the use of chemical reagents that generate a detectable signal that corresponds to the level of antibody binding, and, accordingly, to the level of biomarker protein expression. For example, antibody binding can be detected through the use of a secondary antibody that is conjugated to a labeled polymer. Examples of labeled polymers include but are not limited to polymer-enzyme conjugates. The enzymes in these complexes are typically used to catalyze the deposition of a chromogen at the antigen-antibody binding site, thereby resulting in cell or tissue staining that corresponds to expression level of the biomarker of interest. Enzymes of particular interest include horseradish peroxidase (HRP) and alkaline phosphatase (AP). Commercial antibody detection systems, such as, for example the Dako Envision+system (Glostrup, Denmark) and Biocare Medical's Mach 3 system (Concord, Calif.), can be used to practice the present invention.

The terms “antibody” and “antibodies” broadly encompass naturally occurring forms of antibodies and recombinant antibodies such as single-chain antibodies, chimeric and humanized antibodies and multi-specific antibodies as well as fragments and derivatives of all of the foregoing, which fragments and derivatives have at least an antigenic binding site. Antibody derivatives may comprise a protein or chemical moiety conjugated to the antibody. The antibodies used to practice the invention are selected to have specificity for the biomarker proteins of interest. Methods for making antibodies and for selecting appropriate antibodies are known in the art. See, for example, Celis, ed. (2006) Cell Biology: A Laboratory Handbook, 3rd edition (Elsevier Academic Press, New York). In some embodiments, commercial antibodies directed to specific biomarker proteins can be used to practice the invention. The antibodies of the invention can be selected on the basis of desirable staining of histological samples. That is, the antibodies are selected with the end sample type (e.g., formalin-fixed, paraffin-embedded head and neck tumor tissue samples) in mind and for binding specificity.

Detection of antibody binding can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, and acetylcholinesterase. Examples of suitable prosthetic group complexes include streptavidin/biotin and avidin/biotin. Examples of suitable fluorescent materials include umbelliferone, fluorescein, fluorescein isothiocyanate, rhodamine, dichlorotriazinylamine fluorescein, dansyl chloride, and phycoerythrin. An example of a luminescent material is luminol Examples of bioluminescent materials include luciferase, luciferin and aequorin. Examples of suitable radioactive materials include ¹²⁵I, ¹³¹I, ³⁵S, and ³H.

In regard to detection of antibody staining in the immunohistochemistry methods of the invention, there also exist in the art, video-microscopy and software methods for the quantitative determination of an amount of multiple molecular species (e.g., biomarker proteins) in a biological sample where each molecular species present is indicated by a representative dye marker having a specific color. Such methods are also known in the art as colorimetric analysis methods. In these methods, video-microscopy is used to provide an image of the biological sample after it has been stained to visually indicate the presence of a particular biomarker of interest. See, for example, U.S. Pat. Nos. 7,065,236 and 7,133,547, which disclose the use of an imaging system and associated software to determine the relative amounts of each molecular species present based on the presence of representative color dye markers as indicated by those color dye markers' optical density or transmittance value, respectively, as determined by an imaging system and associated software. These techniques provide quantitative determinations of the relative amounts of each molecular species in a stained biological sample using a single video image that is “deconstructed” into its component color parts.

Proteomics

The term “proteome” is defined as the totality of the proteins present in a sample (e.g., tissue, organism or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE) or liquid/gas chromatography; (2) identification of the individual proteins recovered from the gel or contained within a column fraction, for example, by mass spectrometry or N-terminal sequencing, and (3) analysis of the data using bioinformatics. Proteomics methods are valuable supplements to other methods of gene expression profiling, and can be used, alone or in combination with other methods, to detect the products of the biomarkers of the present invention.

Kits

Kits for practicing the methods of the invention are further provided. By “kit” is intended any manufacture (e.g., a package or a container) including at least one reagent, such as a nucleic acid probe, an antibody or the like, for specifically detecting the expression of a biomarker of the invention. The kits can be promoted, distributed or sold as units for performing the methods of the present invention. Additionally, kits can contain a package insert describing the kit and methods for its use.

In particular embodiments, kits for diagnosing and for evaluating the prognosis of a head and neck cancer patient including detecting biomarker overexpression at the nucleic acid level are provided. Such kits are compatible with both manual and automated nucleic acid detection techniques (e.g., gene arrays). These kits include, for example, at least five nucleic acid probes that specifically bind to five distinct biomarker nucleic acids or fragments thereof

In other embodiments, kits for practicing the immunohistochemistry methods of the invention are provided. Such kits are compatible with both manual and automated immunohistochemistry techniques (e.g., cell staining). These kits include at least five antibodies for specifically detecting the expression of at least five distinct biomarkers. Each antibody can be provided in the kit as an individual reagent or, alternatively, as an antibody cocktail comprising at least five antibodies directed to at least five different biomarkers.

Any or all of the kit reagents can be provided within containers that protect them from the external environment, such as in sealed containers. Positive and/or negative controls can be included in the kits to validate the activity and correct usage of reagents employed in accordance with the invention. Controls can include samples, such as tissue sections, cells fixed on glass slides, RNA preparations from tissues or cell lines, and the like, known to be either positive or negative for the presence of at least five different biomarkers. The design and use of controls is standard and well within the routine capabilities of those of ordinary skill in the art.

A method of identifying a compound that prevents or treats head and neck cancer, the method comprising the steps of: (a) contacting a tissue or an animal model with a compound; (b) measuring nuclear p16 expression levels; and (c) comparing the nuclear p16 expression levels in the animal model with a level associated with a control; and determining a functional effect of the compound on the bacteria levels, thereby identifying a compound that prevents or treats head and neck cancer.

The article “a” and “an” are used herein to refer to one or more than one (i.e., to at least one) of the grammatical object of the article. By way of example, “an element” means one or more element.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. The present invention may suitably comprise, consist of, or consist essentially of, the steps and/or reagents described in the claims.

The following Examples further illustrate the invention and are not intended to limit the scope of the invention.

### 6. EXAMPLES

### 6.1. Different Cellular p16^{INK4a} Localization May Signal Different Survival Outcomes in Head and Neck Cancer

The Carolina Head and Neck Cancer Study (CHANCE) was a population-based case-control study of incident HNSCC conducted from 2002 to 2006 in 46 counties in Central and Eastern North Carolina (Divaris et al., 2010). The subcohort of 143 patients from this study who were treated at UNC hospitals and had banked tissue available were eligible. Patients with cancers of all head and neck subsites except nasopharynx (oral cavity, oropharynx, larynx and hypopharynx) were included. Treatment decisions were recommended by the UNC Head and Neck multidisciplinary team, and based on patient age, tumor extent, site, comorbidities and performance status. Clinical information was extracted from patient charts. Patients who received complete medical care at UNC were followed by retrospective review of the medical record for outcomes including relapse and death. Patients who had follow up in local institutions outside UNC were followed by requesting medical records from the local institution or in cases where there was no return of information from the outside institution, patients deaths were queried from the Social Security Death Index and local obituaries in compliance with the CHANCE study protocols. Patients without sufficient tumor sample for p16 staining were excluded, leaving 135 patients in the analysis. An independent UNC TMA cohort was available for validation which our group has reported on previously (Harris et al., 2010a).

Tissue Microarray

Tissue microarrays (TMAs) were constructed using core samples from formalin-fixed paraffin-embedded tumor blocks. Hematoxylin and eosin stained slides were reviewed by two pathologists to confirm the original diagnosis. One mm microarray blocks were constructed on a manual tissue microarrayer-1 from Beecher Instruments (Sun Prairie WI 53590) in triplicate. Sequential four micrometer sections were cut from each tissue microarray. Sectioned slides were coated in paraffin and stored at 4° C. until staining. A second confirmatory tissue resource was also used for the current analysis the construction and results of which have been previously reported (Harris et al., 2010a). Briefly, a TMA (designated young nonsmoking oral cavity cohort, YNOCC) was constructed in a similar manner as above that included a cohort of 42 HNSCC between the age of 18 and 39. Processing of tissue and reagents is otherwise consistent with the current methods.

p16 Immunohistochemical Staining (IHC)

p16 IHC staining was carried out in the Bond Autostainer (Leica Microsystems Inc, Norwell Mass. 02061) according to the manufacturer's IHC protocol. Slides were put in a 60 degree oven to remove excess paraffin. Slides were then placed in the autostainer and dewaxed in Bond Dewax solution (AR9222) and hydrated in Bond Wash solution (AR9590). Antigen retrieval was performed for 30 min at 100° C. in Bond-Epitope Retrieval solution 1 (pH 6.0, AR9961). Slides were then incubated with p16INK4a antibody (mouse monoclonal anti-p16 antibody (MAB4133), Chemicon® International Company/Millipore Corporation, Temecula Calif. 92590) for 15 minutes. Antibody detection was performed using the Bond Polymer Refine Detection System (DS9800). Stained slides were dehydrated and coverslips added. IHC was performed in the Translational Pathology Lab at UNC. After completion of IHC, slides are stored at room temperature in our laboratory and a virtual scanned copy of all TMA slides will be kept for further reference.

HPV In Situ Hybridization

HPV in situ hybridization was carried out in Ventana Benchmark XT autostainer. Slide deparaffinization, conditioning, and staining with INFORM HPV III Family 16 Probe (B; Ventana Medical Systems) were performed on the autostainer according to the manufacturer's protocol. The probes have affinities to HPV subtypes 16, 18, 31, 33, 35, 39, 45, 51, 52, 56, 58 and 66. Slides were scored as positive for HPV if a punctate or diffuse pattern of signal was observed in the tumor nuclei.

p16 Protein Expression

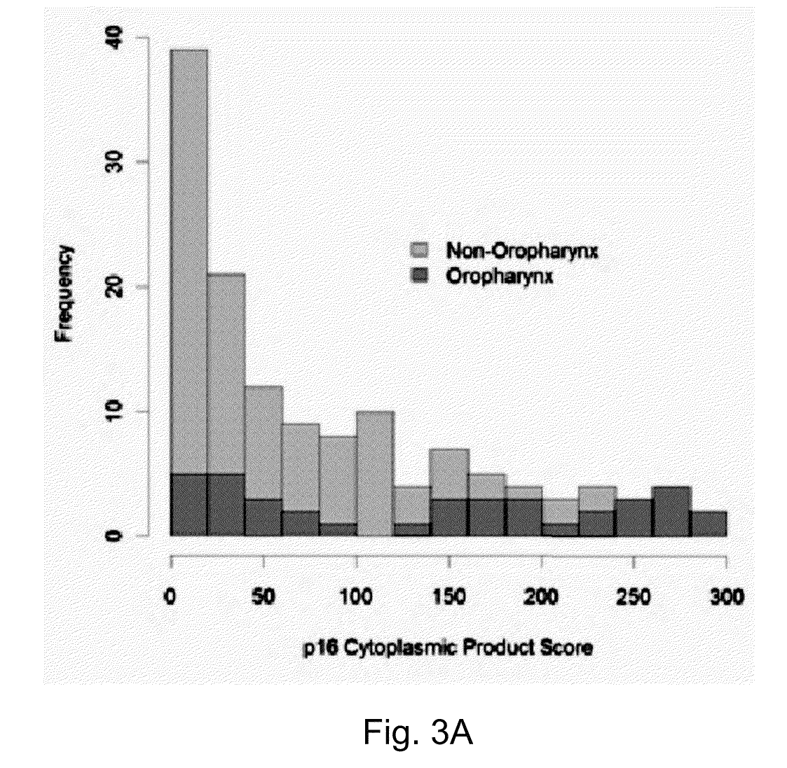

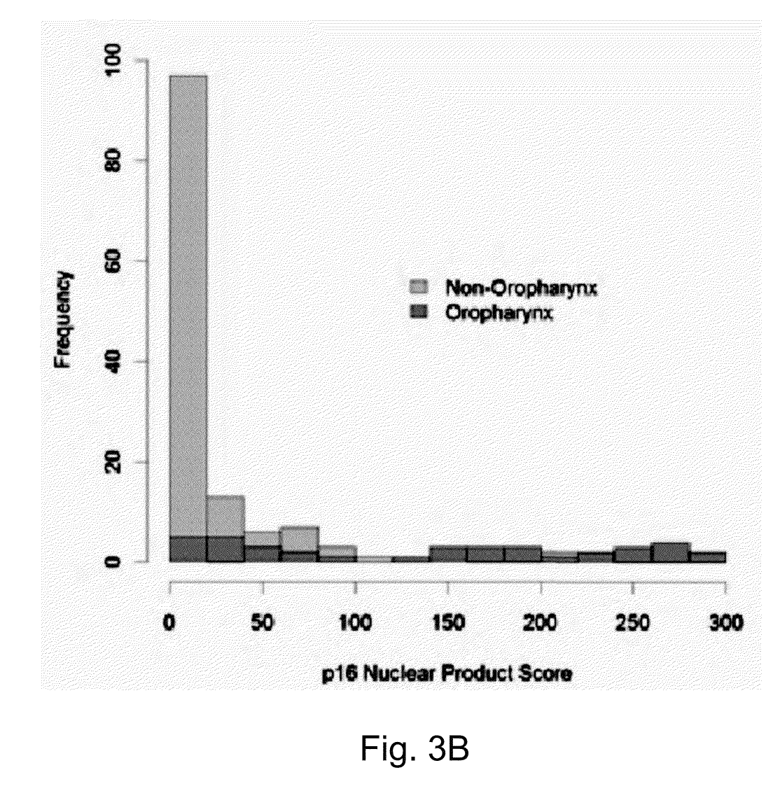

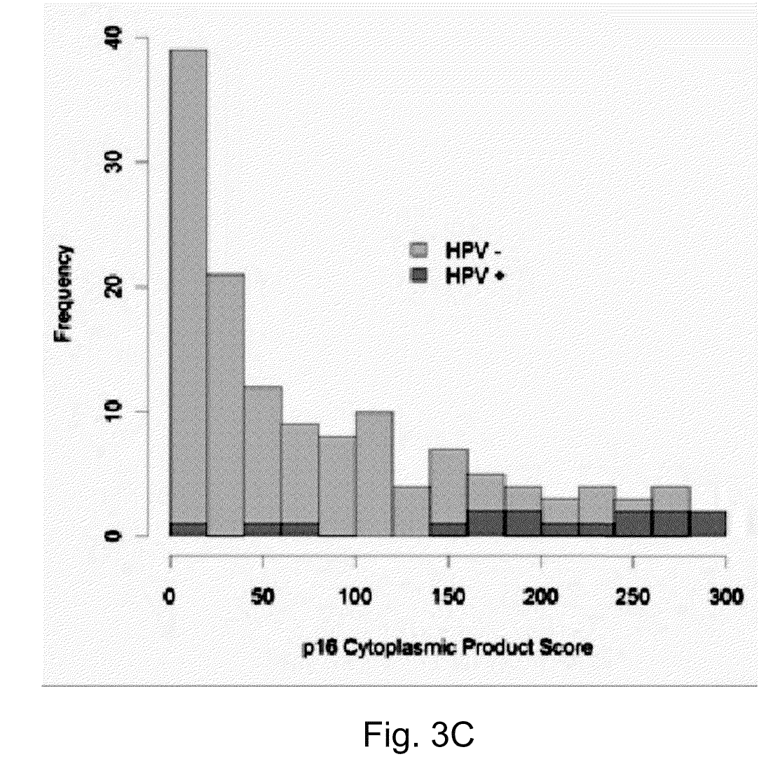

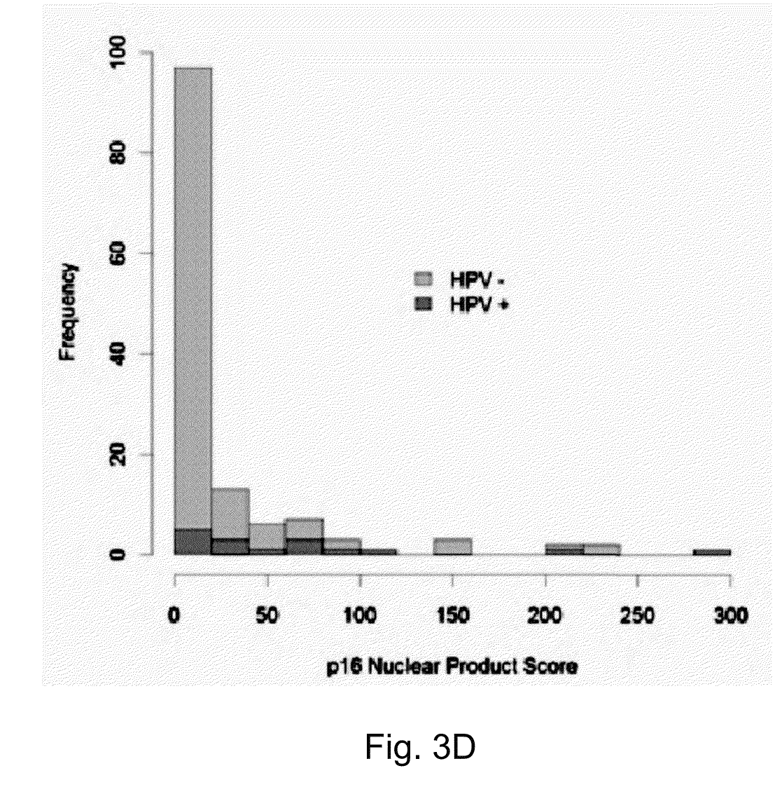

p16 expression was assessed by pathologists who were blinded as to the clinical data for the patients. The CHANCE TMA and the YNOCC TMA were read by two pathologists, with any indeterminate scores evaluated by a third pathologist. Digital images of cells were captured (magnification×200) using Aperio Scanscope. Tissue samples previously shown to be p16 overexpressors (endometrium) were used as a positive control for intensity scoring. Each sample was given a cytoplasmic intensity score and nuclear intensity score on a scale of 0 to 3, with intensity scored 0 equal to no staining; 1, faint or focal cytoplasmic staining; 2, moderate, diffuse staining; 3, intense and diffuse staining. The percent of tumor cells with positive nuclei was determined by scoring 10 microscopic fields of 100 tumor cells each. A semi-quantitative percentage score was generated for cytoplasm and nucleus staining for each specimen, ranging from 0 to 100. The TMA was constructed with the goal to obtain 3 cores per patient block. Not every block had sufficient tissues and some cases resulted with only one or two cores. For samples that had multiple cores, mean intensity or percentage scores across the cores were used as the final intensity or percentage score for that sample. A composite product score was calculated by multiplying the mean intensity score and mean percentage score in cytoplasm or nucleus. Based on a bimodal distribution of the scores in oropharynx patients (dark grey in FIG. 3), a nuclear product score of 100 was used as a cutoff for nuclear staining. The 75% percentile of cytoplasmic staining (133.4) was considered to be a cutoff for cytoplasmic staining. All samples that had high nuclear staining also had high cytoplasmic staining, resulting in three categories in total. Patients with a nuclear product score ≧100 were considered high nuclear staining (HN). Patients with a product score at or above the 75th percentile of the cytoplasmic score (133.4) were considered high cytoplasmic staining (HC) if they were not in the HN group. Patients who failed to meet criteria either for high nuclear or high cytoplasmic score were categorized in the low staining group (LS). Based on this empirical separation, the patients were divided into three groups; high nuclear, any cytoplasmic staining (HN), high cytoplasmic, low nuclear staining (HC), and low nuclear and cytoplasmic staining (LS).

Statistical Analysis

All statistical analysis was performed using R 2.9.2 software (http://cran.r-project.org). Baseline characteristics of patients from each group (HN, HC, LS) were compared using Fisher's exact test for categorical variables and one way analysis of variance (ANOVA) for continuous variables. Overall survival (OS) was calculated as the time from diagnosis date to death date or the last documented follow-up date. Progression-free survival (PFS) was defined as the time from diagnosis date to the date of disease progression or the last documented follow-up date or death date from any cause. Disease progression was defined as any documented tumor progression (local or distant) as indicated in the clinical record. All observations were censored at 60 months. Survival curves were calculated using the Kaplan-Meier method and compared non-parametrically using the log-rank test. Cox proportional hazard model was used to estimate the hazard ratio between different p16 staining groups, adjusting for patient drinking status, tumor stage, tumor site and HPV staining. All statistical tests were two sided with a significance level of 0.05 and all reported confidence intervals are constructed at a two sided 95% confidence level.

Results

Patient Characteristics

143 patients were identified during the study period, of which 135 had sufficient tumor samples for p16 staining. The median follow up time for these patients was 6.67 years, with only 5 patients lost to follow up before 5 years. The baseline characteristics for these patients were summarized in Table 1. The median age of patients at diagnosis was 57 (range 20-82). 68.9% of the patients were males, which is comparable to the national average (Ries LAG, 2007). Most patients had smoking histories and/or alcohol use with only 12 (9%) never-smokers and 44 (approximately 30%) never-drinkers. Furthermore, all of the 123 smokers, except two, had smoked more than 10 pack years. Approximately 30% of the patients received single modality treatment with surgery or radiation alone. Other patients received a combination of different treatment methods. Sixteen (11.9%) patients were detected as HPV positive, of which 14 had oropharyngeal tumors and the other two had tumors in the oral cavity.

p16 Expression

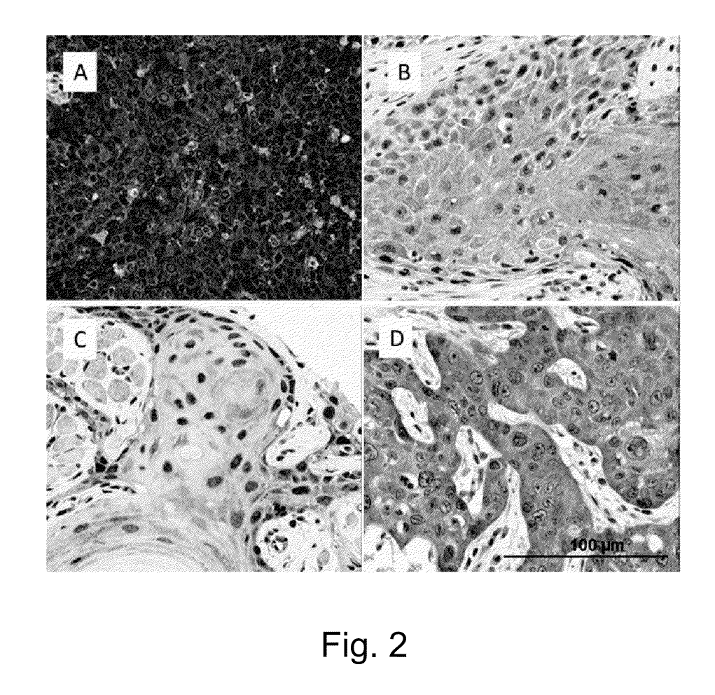

In the sample set p16 showed baseline cytoplasmic and nuclear staining in at least one of the three cores for every patient. Examples of IHC images of p16 staining are shown in FIG. 2. Overall, oropharyngeal cancers and HPV-positive cancers had stronger p16 staining in both cytoplasm and nucleus compared to tumors of other types (FIG. 3). The median nuclear product score was 22 in oropharyngeal tumor samples compared with 0 in non-oropharyngeal samples (permutation test of equal density p-value <0.001). The median cytoplasmic product score was 150 in oropharyngeal tumor samples compared to a median product score of 38 in non-oropharyngeal samples (permutation test of equal density p-value <0.001). Nine patients had high nuclear and high cytoplasmic p16 staining (HN), 25 patients had high cytoplasmic, low nuclear staining (HC) and 101 had low p16 staining (LS). There was no significant difference in age, gender, smoking status, T stages and clinical stages between different staining groups. However, patients with high nuclear or cytoplasmic p16 staining have more oropharyngeal tumors and earlier nodal stage (N0-N1) compared to low p16 staining group.

HPV In Situ Hybridization

Table 2 summarized the distribution of tumor sites with respect to HPV positivity and smoking status. Overall, 16 of the 143 patients stained positively for HPV, with fourteen of them having tumors in oropharynx and two in the oral cavity. The HPV positivity rates were lower than some of the clinical trials and other university based reports (Chuang et al., 2008; Fakhry et al., 2008), due to, at least in part, the very high smoking rate in our study population (Ang et al., 2010; D'Souza et al., 2007). 58% ( 14/24) of oropharyngeal tumors were stained HPV positive in this study, comparable to previous reports such as D'Souza and colleagues (D'Souza et al., 2007), which reported 64% HPV positive in oropharyngeal cancers. HPV positive staining outside oropharyngeal tumors was rare, which is consistent with the general acceptance of a low rate of HPV infection outside the oropharynx (Begum et al., 2007). The vast majority of these HPV positive patients were heavy smokers: 13 of the 16 HPV-infected patients had long histories of smoking, with a minimum of 18 pack years. HPV infection has been strongly associated with both cytoplasmic and nuclear p16 positivity. All but three HPV positive patients were categorized as having high nuclear or high cytoplasmic p16 expression.

Survival Analysis

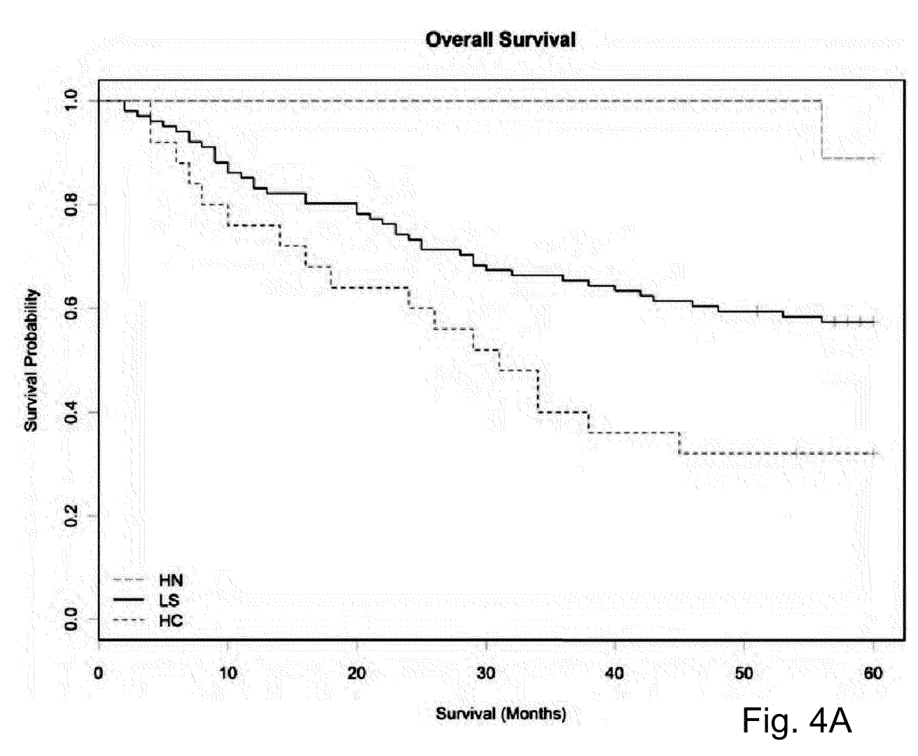

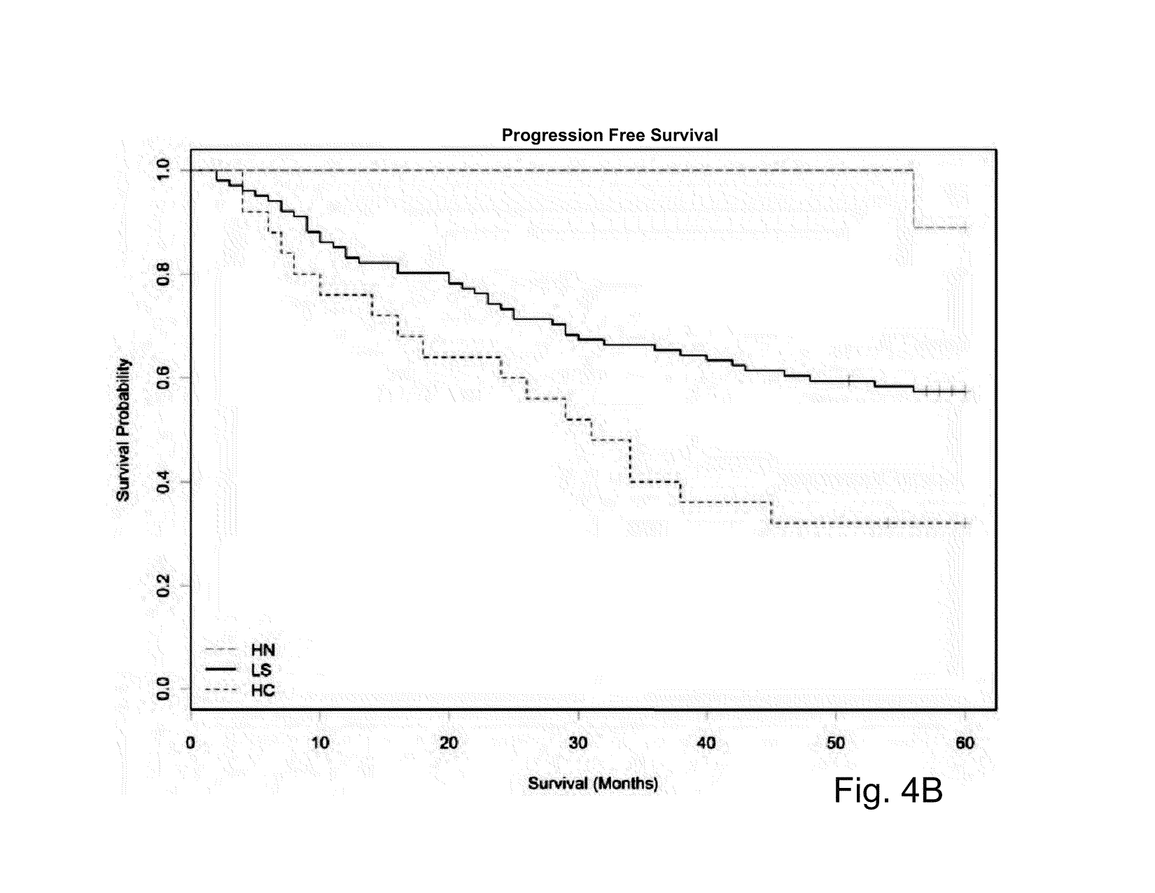

In the full cohort, the three-year overall survival (OS) was 63.0% (95% CI: 55.3%-71.7%) and the three-year progression-free survival (PFS) rate was 54.1% (95% CI: 46.3%-63.2%). Only one death occurred in HN group during the follow up. In the LS group, the three year OS and PFS was estimated as 65.3% (95% CI: 56.7%-75.3%) and 54.5% (95% CI: 45.6%-65.1%) using the Kaplan Meier method. The three year OS and PFS was estimated as 40% (95% CI: 24.7%-64.6%) and 36% (95% CI: 21.3%-60.7%) respectively in the HC group (FIG. 4). The 3 year OS and PFS survival in the HN group was 100% with confidence interval not evaluable. Both OS and PFS results were significantly different between staining groups with a log rank test p values of 0.006 and 0.009 respectively. There is no significant difference in OS or PFS between HPV positive group and HPV negative group (p=0.509 and 0.434 respectively).

Cox proportional hazard model was used to assess the relationship between each variable with OS and PFS (Table 3). p16 expression status was significantly associated with both OS and PFS. The HN group had the best overall survival outcome and the lowest hazard ratio compared with the other groups. Similar results were obtained for progression-free survival, although the difference was not statistically significant. Using the HC group as a reference, the hazard ratio was 0.50 (95% CI 0.29-0.88) for the LS group and 0.10 (95% CI 0.013-0.75) for the HN group. Similarly, the hazard ratio for progression-free survival was 0.61 (95% CI 0.35-1.04) in the LS group and 0.09 (95% CI 0.012-0.67) in the HN staining group. If we consider local recurrence and distant recurrence separately, the three year local recurrence rate and distant relapse rates were 24% and 26.7% for HC and LS group respectively, and the three year distant recurrence rate was 16.0% and 10.9% for HC and LS group, respectively. HN group had no recurrence during three years of follow up. When nuclear staining and cytoplasmic staining were considered separately for their association with OS or PFS, high nuclear staining was significantly associated with PFS (HR=0.13, 95% CI 0.018-0.96) and insignificantly associated with OS (HR=0.17, 95% CI 0.024-1.24). Cytoplasmic staining was not significantly associated with either OS or PFS. In addition to p16 staining status, T3-T4 tumor stage was significantly associated with increased risk of mortality (p-value=0.009). Nodal stages showed borderline significance in affecting overall survival (p-value=0.07). No variable tested except p16 expression status showed significant association with PFS.

Multivariable Cox proportional hazard model showed that p16 expression status was still significantly associated with both OS and PFS (Table 4) after adjusting for tumor site, nodal stage, tumor stage HPV staining and drinking pattern. Both the LS group and the HN staining group had significantly lower hazard than the HC staining group. Subset analysis was carried out for oropharynx patients: after controlling for tumor stages, HPV staining and drinking status, the hazard ratio of OS for LS and HN groups are 0.40 (p=0.18) and 0.12 (p=0.06) respectively, and the hazard ratio of PFS for LS and HN groups are 0.61 (p=0.43) and 0.12 (p=0.06) respectively, using the HC group as reference. Subset analysis for other tumor sites was not conducted because of the small number of patients.

Independent Confirmation in Second Cohort

Using data from the YNOCC TMA, we were able to obtain p16 staining on an additional 42 samples, with 30 from the oral cavity, 6 from the oropharynx, 5 from the larynx and 1 from the hypopharynx. This is a cohort of younger patients who were diagnosed between the age of 20 and 39, with 23 males, 29 with smoking history (median pack year 14.5) and 18 with alcohol consumption history. Previously we had reported a favorable overall outcome for those patients in the cohort who were p16 positive. At that time, we had not evaluated the independent contribution of nuclear staining to outcomes. In this study, we evaluated those patients by the same product score cutoff s an independent validation. The patients were then grouped using the same criteria for this study: 14 patients were placed in the HN group, 4 patients in the HC group and 24 patients in the LS group. Although p values are not statistically significant due to small sample size, strikingly, the HN staining group had superior progression-free survival compared with the other two groups, with similar magnitude to our observations in the CHANCE data set. The hazard ratio of having a recurrence in the HN group and LS group are 0.38 (95% CI 0.092-1.62) and 0.71 (95% CI 0.20-2.52) compared to the HC staining group (p=0.34).

### DISCUSSION

The management of squamous cell carcinoma of the head and neck appears to be at a crossroads, with the possibility that the field may change long held treatment standards based on observations related to the staining for the biomarkers HPV and p16. Pivotal studies have documented significantly improved outcomes for patients staining positively for these markers, yet a closer look at how these biomarkers relate to each other has stimulated researchers to look for the mechanisms behind the beneficial outcome association. Firstly, it is clear that mechanisms in addition to HPV infection itself are at work as evidenced by the modulation of risk caused by smoking. There is also at least circumstantial evidence that alterations of p16, independent of HPV, may convey some of the favorable prognosis seen in HNSCC patients that cannot simply be ascribed to false negative HPV assays. Evidence from tumors outside the head and neck lead us to consider nuclear localization of p16 as a novel biomarker. In this report, the results comparing nuclear localization of p16 to cases where p16 is excluded from the nucleus warrant further study. Furthermore, the results may help suggest a mechanistic role for this biomarker that go beyond an empiric view of p16 as a proxy for HPV of use limited to the oropharynx.

To consider p16 status (as indicated by p16 staining) as a mechanistic marker requires a review of the ways that p16 is altered in cancer. In the case of HPV, p16 overexpression is a result of expression of HPV-derived oncoproteins E6 and E7 and can functionally inactivate the p53 and pRb tumor suppressor protein, resulting in a down-regulation of p53, pRb and a strong up-regulation of p16 at the molecular level (Andl et al., 1998; Li et al., 2004; Marur et al., 2010; Wiest et al., 2002). One could think of p16 expression in the context of HPV infection as a proxy for multiple genotypes that would generally be considered favorable for cancer prognosis (p53 wild type (WT), Rb WT, and p16 WT). However, in the more common setting of tumors, p16 is lowly expressed, possibly by less favorable genetic or epigenetic changes, such as homozygous deletion of p16, nonsense mutation, or perhaps methylation and gene silencing. In those situations, where there are more deleterious mutations such as loss of Rb or perhaps amplification of cyclin D1 (common in HNSCC), the tumors can express high levels of p16 with no inhibition of cell cycling. In these situations, nuclear trafficking might be altered and high p16 expression might indicate particularly unfavorable cancer biology. Smoking could be the means of inactivation of genes downstream of p16 without requiring p16 loss as the disease modifying event associated with worse outcome. To evaluate such an explanation, we attempted to sequence p16 and other targets in the current sample set but were unsuccessful due to the quality of the DNA in these paraffin embedded specimens.

To our knowledge, no previous study has investigated how different p16 expression localization can be related to disease outcomes in HNSCC despite evidence that differential staining patterns similar to what we describe have been shown to be relevant in other tumors, including endometrial cancers, melanoma and astrocytomas (Arifin et al., 2006; Emig et al., 1998; Ghiorzo et al., 2004; Milde-Langosch et al., 2001; Salvesen et al., 2000; Straume et al., 2000). Most strikingly, familial melanoma studies strongly support our hypothesis because of the associated point mutations and the failure to localize p16 to the nucleus (Ghiorzo et al., 2004). In this report, patients without the germline variant displayed a combined nuclear and cytoplasmic staining. The authors demonstrated that p16 mutations in these melanoma patients may impair the cytoplasmic-nuclear shuttling similar to BRCA1 where BRCA1 is shifted to the cytoplasm because of the mutation of nuclear localization signals (NLS) and the HN2-terminal (Arifin et al., 2006; Fabbro et al., 2004; Ghiorzo et al., 2004).

The current study includes limitations that suggest further evaluation of p16 nuclear staining is warranted. Most notably, the current study is relatively small and includes a large number of smokers. Similarly, due to the retrospective nature of the current study, patients are heterogeneous in stage, site, treatment, and other factors that might impact risk in ways that have not been appreciated. However, the prognostic effect of p16 localization remained significant after controlling for these factors. The validation cohort provided extra support for our result. We do provide evidence regarding the use of p16 in nonsmokers with the YNOCC cohort, but this group does not include significant numbers of nonsmoking HPV positive patients. However, because most HNSCC patients are still smokers despite the rising numbers of non-smoking patients, these data are applicable to a larger portion of HNSSC patients. Finally, our cutoff for different p16 groups was based on the empirically observed distributions of p16 staining in oropharynx versus non-oropharynx samples. This cutoff was neither optimized nor cross-validated and cannot be directly used for clinical settings.

In conclusion, we have provided a preliminary investigation into the nuclear staining of p16 as a critical factor in the complex set of conditional biomarkers including HPV, smoking, oropharyngeal carcinomas, and non-localized staining of p16. This biomarker, if validated, is already widely available and could potentially impact clinical care of HNSCC. See also Zhao et al. 2012 Brit J Cancer 107 482-490 (pub. online 2012 Jun. 26) the contents of which are hereby incorporated in their entity.

TABLE 1
Patient characteristics by p16 staining.

| | p16 staining groups | | | | |
|---|---|---|---|---|---|
| | All patients | HN | HC | LS | |
| Characteristics | (column %) | (column %) | (column %) | (column %) | P values |
| # of patients | 135 | 9 | 25 | 101 | |
| Age | | | | | |
| Median | 57 | 56 | 54 | 58 | 0.14 |
| Range | 20-82 | 20-66 | 34-79 | 24-82 | |
| Gender | | | | | |
| Male | 93 (68.9) | 8 (88.9) | 19 (76) | 66 (65.3) | 0.28 |
| Smoking* | 123 (91.1) | 7 (77.8) | 22 (88) | 94 (93.1) | 0.16 |
| Mean pack years (SD)* | 39.8 (25.9) | 41.4 (39.1) | 38.0 (26.0) | 40.0 (24.7) | 0.93 |
| Alcohol* | 91 (67.4) | 6 (66.7) | 18 (72) | 67 (66.3) | 0.90 |
| T stage* | | | | | |
| T1-T2 | 65 (48.1) | 4 (44.4) | 10 (40) | 51 (50.5) | 0.65 |
| T3-T4 | 70 (51.9) | 5 (55.6) | 15 (60) | 50 (49.5) | |
| Nodal stage* | | | | | |
| N0-N1 | 79 (58.5) | 4 (44.4) | 8 (32) | 67 (66.3) | 0.004 |
| N2-N3 | 56 (41.5) | 5 (55.6) | 17 (68) | 34 (33.7) | |
| Stage | | | | | |
| Stage I-II | 43 (31.9) | 1(11.1) | 5 (20) | 37 (36.6) | 0.12 |
| Site | | | | | |
| Oropharynx | 38 (28.1) | 7 (77.8) | 15 (60) | 16 (15.8) | <0.001 |
| Larynx | 35 | 1 (11.1) | 1 (4) | 33 (2.7) | |
| Oral cavity | 54 (40) | 1 (11.1) | 6 (24) | 47 (46.5) | |
| Hypopharynx | 8 (5.9) | 0 | 3 (12) | 5 (5.0) | |
| HPV | | | | | |
| Positive | 16 (11.9) | 3 (33.3) | 10 (40.0) | 3 (3.0) | <0.001 |

*Numbers do not sum to the total due to missing data Abbreviations:
HN, high nuclear, any cytoplasmic staining;
HC, high cytoplasmic, low nuclear staining;
LS, low nuclear,low cytoplasmic staining

TABLE 2
p16 expression by smoking status and tumor site

| p16 Expression | HN | HC | LS |
|---|---|---|---|
| Smokers | | | |
| HPV negative | 4 (1 OC, 3 OP) | 14 (5 OC, 1 LA, | 92 (40 OC, 33 LA, |
| | | 3 HY, 5 OP) | 3 HY, 16 OP) |
| HPV positive | 3 (OP) | 8 (OP) | 2 (1 OC, 1OP) |
| Nonsmokers | | | |
| HPV negative | 2 (OP) | 1 (OC) | 6 (5 OC, 1 OP) |
| HPV positive | 0 | 2 (OP) | 1 (1 OC) |

Abbreviations: HPV, human papillomavirus; OC, oral cavity; LA, larynx; HY, hypopharynx; OP, Oropharynx; HN, high nuclear, any cytoplasmic staining; HC, high cytoplasmic, low nuclear staining; LS, low nuclear, low cytoplasmic staining

TABLE 3
Univariate analyses of prognostic factors for overall or progression-free survival

| | PFS | | | | | OS | | | | |
|---|---|---|---|---|---|---|---|---|---|---|
| | | | | | P | | | | | P |
| Characteristics | #events | PYs | HR | 95% CI | value | #events | PYs | HR | 95% CI | value |
| Age (Years) | | | | | | | | | | |
| ≧57/<57 | 38/34 | 228/197 | 0.97 | 0.61-1.55 | 0.91 | 33/28 | 254/224 | 1.03 | 0.62-1.70 | 0.92 |
| Smoker/non- | 66/6 | 385/41 | 1.19 | 0.52-2.74 | 0.69 | 56/5 | 433/45 | 1.19 | 0.48-2.97 | 0.71 |
| smoker | | | | | | | | | | |
| Drinking | 53/19 | 270/154 | 1.55 | 0.92-2.62 | 0.10 | 45/16 | 305/174 | 1.61 | 0.91-2.84 | 0.10 |
| Site | | | | | | | | | | |
| Larynx | 18 | 111 | 1.17 | 0.61-2.25 | 0.63 | 14 | 134 | 1.01 | 0.49-2.10 | 0.97 |
| Oral Cavity | 29 | 166 | 1.27 | 0.71-2.29 | 0.42 | 26 | 179 | 1.40 | 0.74-2.64 | 0.30 |
| Hypopharynx | 7 | 15.4 | 2.74 | 1.14-6.60 | 0.02 | 6 | 20 | 2.70 | 1.04-6.99 | 0.04 |
| Oropharynx | 18 | 131 | 1.0 (reference) | | | 15 | 145 | 1.0 (reference) | | |
| T stage | | | | | | | | | | |
| T3-T4/T1-T2 | 42/30 | 202/221 | 1.49 | 0.93-2.38 | 0.10 | 39/22 | 220/258 | 2.02 | 1.20-3.41 | 0.009 |
| N stage | | | | | | | | | | |
| N2-N3/N0-N1 | 31/41 | 165/259 | 1.16 | 0.73-1.86 | 0.52 | 30/31 | 177/301 | 1.61 | 0.97-2.65 | 0.07 |
| Stage | | | | | | | | | | |
| Late(III-IV)/ | 52/20 | 281/144 | 1.29 | 0.77-2.17 | 0.33 | 47/14 | 309/169 | 1.79 | 0.98-3.25 | 0.06 |
| Early(I-II) | | | | | | | | | | |
| p16: combined | | | | | | | | | | |
| nuclear and | | | | | | | | | | |
| cytoplasmic staining | | | | | | | | | | |
| HN | 1 | 45 | 0.09 | 0.012-0.67 | 0.067 | 1 | 45 | 0.10 | 0.013-0.75 | 0.025 |
| LS | 53 | 319 | 0.61 | 0.35-1.04 | 0.019 | 43 | 365 | 0.50 | 0.29-0.88 | 0.017 |
| HC | 18 | 61 | 1.0 (reference) | | | 17 | 68 | 1.0 (reference) | | |
| HPV | | | | | | | | | | |
| +/− | 7/65 | 55/368 | 0.73 | 0.34-1.60 | 0.44 | 6/22 | 60/418 | 0.75 | 0.32-1.75 | 0.51 |

Abbreviations: PYs: person-years; PFS, progression-free survival; OS, overall survival; HR, hazard ratio; CI, confidence interval; LS, Low nuclear, low cytoplasmic staining; HN, high nuclear, high cytoplasmic staining; HC, high cytoplasmic, low nuclear staining

TABLE 4
Multivariate analysis of prognostic factors
for survival or progression-free survival

| | PFS | | | OS | | |
|---|---|---|---|---|---|---|
| | | | P | | | P |
| | | | val- | | | val- |
| | HR | 95% CI | ues | HR | 95% CI | ues |
| T3-T4/T1-T2 | 1.32 | 0.77-2.26 | 0.31 | 1.72 | 0.94-3.12 | 0.08 |
| N2-N3/N0-N1 | 0.96 | 0.55-1.68 | 0.90 | 1.24 | 0.68-2.28 | 0.48 |
| Drinking | 1.37 | 0.76-2.49 | 0.29 | 1.21 | 0.64-2.31 | 0.56 |
| Site | | | | | | |
| Larynx | 1.29 | 0.57-2.89 | 0.54 | 1.34 | 0.54-3.30 | 0.53 |
| Oral Cavity | 1.35 | 0.66-2.78 | 0.41 | 1.65 | 0.75-3.60 | 0.21 |
| Hypopharynx | 1.73 | 0.66-4.56 | 0.27 | 1.69 | 0.59-4.84 | 0.33 |
| Oropharynx | 1(reference) | | | 1(reference) | | |
| p16 staining | | | | | | |
| HN | 0.092 | 0.01-0.71 | 0.02 | 0.10 | 0.01-0.78 | 0.03 |
| LS | 0.475 | 0.24-0.95 | 0.03 | 0.37 | 0.18-0.75 | 0.01 |
| HC | 1 (reference) | | | 1 (reference) | | |
| HPV Positives | 0.65 | 0.24-1.81 | 0.41 | 0.54 | 0.179-1.61 | 0.27 |

Abbreviations: PFS, progression-free survival; OS, overall survival; HR, hazard ratio; CI, confidence interval of hazard ratio; HN, high nuclear, high cytoplasmic staining; HC, high cytoplasmic, low nuclear, staining; LS, Low nuclear, low cytoplasmic staining

### 6.2. References (Sec. 2.1 and 6.1)

Allred, D. C., Harvey, J. M., Berardo, M. & Clark, G. M. (1998). Prognostic and predictive factors in breast cancer by immunohistochemical analysis. Mod Pathol, 11, 155-68.
Andl, T., Kahn, T., Pfuhl, A., Nicola, T., Erber, R., Conradt, C., Klein, W., Helbig, M., Dietz, A., Weidauer, H. & Bosch, F. X. (1998). Etiological involvement of oncogenic human papillomavirus in tonsillar squamous cell carcinomas lacking retinoblastoma cell cycle control. Cancer Res, 58, 5-13.
Ang, K. K., Harris, J., Wheeler, R., Weber, R., Rosenthal, D. I., Nguyen-Tan, P. F., Westra, W. H., Chung, C. H., Jordan, R. C., Lu, C., Kim, H, Axelrod, R., Silverman, C. C., Redmond, K. P. & Gillison, M. L. (2010). Human papillomavirus and survival of patients with oropharyngeal cancer. N Engl J Med, 363, 24-35.
Arifin, M. T., Hama, S., Kajiwara, Y., Sugiyama, K., Saito, T., Matsuura, S., Yamasaki, F., Arita, K. & Kurisu, K. (2006). Cytoplasmic, but not nuclear, p16 expression may signal poor prognosis in high-grade astrocytomas. J Neurooncol, 77, 273-7.
Begum, S., Gillison, M. L., Nicol, T. L. & Westra, W. H. (2007). Detection of human papillomavirus-16 in fine-needle aspirates to determine tumor origin in patients with metastatic squamous cell carcinoma of the head and neck. Clin Cancer Res, 13, 1186-91.
Chaturvedi, A. K., Engels, E. A., Pfeiffer, R. M., Hernandez, B. Y., Xiao, W., Kim, E., Jiang, B., Goodman, M. T., Sibug-Saber, M., Cozen, W., Liu, L., Lynch, C. F., Wentzensen, N., Jordan, R. C., Altekruse, S., Anderson, W. F., Rosenberg, P. S. & Gillison, M. L. (2011). Human Papillomavirus and Rising Oropharyngeal Cancer Incidence in the United States. J Clin Oncol.
Chuang, A. Y., Chuang, T. C., Chang, S., Zhou, S., Begum, S., Westra, W. H., Ha, P. K., Koch, W. M. & Califano, J. A. (2008). Presence of HPV DNA in convalescent salivary rinses is an adverse prognostic marker in head and neck squamous cell carcinoma. Oral Oncol, 44, 915-9.
Curado, M. P. & Hashibe, M. (2009). Recent changes in the epidemiology of head and neck cancer. Curr Opin Oncol, 21, 194-200.
D'Souza, G., Kreimer, A. R., Viscidi, R., Pawlita, M., Fakhry, C., Koch, W. M., Westra, W. H. & Gillison, M. L. (2007). Case-control study of human papillomavirus and oropharyngeal cancer. N Engl J Med, 356, 1944-56.
Dahlstrand, H., Dahlgren, L., Lindquist, D., Munck-Wikland, E. & Dalianis, T. (2004). Presence of human papillomavirus in tonsillar cancer is a favourable prognostic factor for clinical outcome. Anticancer Res, 24, 1829-35.
Divaris, K., Olshan, A. F., Smith, J., Bell, M. E., Weissler, M. C., Funkhouser, W. K. & Bradshaw, P. T. (2010). Oral health and risk for head and neck squamous cell carcinoma: the Carolina Head and Neck Cancer Study. Cancer Causes Control, 21, 567-75.
El-Mofty, S. K. & Lu, D. W. (2003). Prevalence of human papillomavirus type 16 DNA in squamous cell carcinoma of the palatine tonsil, and not the oral cavity, in young patients: a distinct clinicopathologic and molecular disease entity. Am J Surg Pathol, 27, 1463-70.
Emig, R., Magener, A., Ehemann, V., Meyer, A., Stilgenbauer, F., Volkmann, M., Wallwiener, D. & Sinn, H. P. (1998). Aberrant cytoplasmic expression of the p16 protein in breast cancer is associated with accelerated tumour proliferation. Br J Cancer, 78, 1661-8.
Fabbro, M., Savage, K., Hobson, K., Deans, A. J., Powell, S. N., McArthur, G. A. & Khanna, K. K. (2004). BRCA1-BARD1 complexes are required for p53Ser-15 phosphorylation and a G1/S arrest following ionizing radiation-induced DNA damage. J Biol Chem, 279, 31251-8.
Fakhry, C., Westra, W. H., Li, S., Cmelak, A., Ridge, J. A., Pinto, H., Forastiere, A. & Gillison, M. L. (2008). Improved survival of patients with human papillomavirus-positive head and neck squamous cell carcinoma in a prospective clinical trial. J Natl Cancer Inst, 100, 261-9.
Franceschi, S., Munoz, N., Bosch, X. F., Snijders, P. J. & Walboomers, J. M. (1996). Human papillomavirus and cancers of the upper aerodigestive tract: a review of epidemiological and experimental evidence. Cancer Epidemiol Biomarkers Prev, 5, 567-75.
Furniss, C. S., McClean, M. D., Smith, J. F., Bryan, J., Nelson, H. H., Peters, E. S., Posner, M. R., Clark, J. R., Eisen, E. A. & Kelsey, K. T. (2007). Human papillomavirus 16 and head and neck squamous cell carcinoma. Int J Cancer, 120, 2386-92.
Ghiorzo, P., Villaggio, B., Sementa, A. R., Hansson, J., Platz, A., Nicolo, G., Spina, B., Canepa, M., Palmer, J. M., Hayward, N. K. & Bianchi-Scarra, G. (2004). Expression and localization of mutant p16 proteins in melanocytic lesions from familial melanoma patients. Hum Pathol, 35, 25-33.
Gillison, M. L., Koch, W. M., Capone, R. B., Spafford, M., Westra, W. H., Wu, L., Zahurak, M. L., Daniel, R. W., Viglione, M., Symer, D. E., Shah, K. V. & Sidransky, D. (2000). Evidence for a causal association between human papillomavirus and a subset of head and neck cancers. J Natl Cancer Inst, 92, 709-20.
Ha, P. K., Pai, S. I., Westra, W. H., Gillison, M. L., Tong, B. C., Sidransky, D. & Califano, J. A. (2002). Real-time quantitative PCR demonstrates low prevalence of human papillomavirus type 16 in premalignant and malignant lesions of the oral cavity. Clin Cancer Res, 8, 1203-9.
Hafkamp, H. C., Manni, J. J., Haesevoets, A., Voogd, A. C., Schepers, M., Bot, F. J., Hopman, A. H., Ramaekers, F. C. & Speel, E. J. (2008). Marked differences in survival rate between smokers and nonsmokers with HPV 16-associated tonsillar carcinomas. Int J Cancer, 122, 2656-64.
Harris, S. L., Kimple, R. J., Hayes, D. N., Couch, M. E. & Rosenman, J. G. (2010a). Never-smokers, never-drinkers: unique clinical subgroup of young patients with head and neck squamous cell cancers. Head Neck, 32, 499-503.
Harris, S. L., Thorne, L. B., Seaman, W. T., Neil Hayes, D., Couch, M. E. & Kimple, R. J. (2010b). Association of p16(INK4a) overexpression with improved outcomes in young patients with squamous cell cancers of the oral tongue. Head Neck.
Jemal, A., Siegel, R., Xu, J. & Ward, E. (2010). Cancer Statistics, 2010. CA Cancer J Clin% R 10.3322/caac.20073, caac.20073.
Li, W., Thompson, C. H., Cossart, Y. E., O'Brien, C. J., McNeil, E. B., Scolyer, R. A. & Rose, B. R. (2004). The expression of key cell cycle markers and presence of human papillomavirus in squamous cell carcinoma of the tonsil. Head Neck, 26, 1-9.
Marur, S., D'Souza, G., Westra, W. H. & Forastiere, A. A. (2010). HPV-associated head and neck cancer: a virus-related cancer epidemic. Lancet Oncol.
Milde-Langosch, K., Bamberger, A. M., Rieck, G., Kelp, B. & Loning, T. (2001). Overexpression of the p16 cell cycle inhibitor in breast cancer is associated with a more malignant phenotype. Breast Cancer Res Treat, 67, 61-70.
National Cancer Institute, T. (2005). http://www.cancer.gov/cancertopics/factsheet/sites-types/head-and-neck.
Patel, S. C., Carpenter, W. R., Tyree, S., Couch, M. E., Weissler, M., Hackman, T., Hayes, D. N., Shores, C. & Chera, B. S. (2011). Increasing incidence of oral tongue squamous cell carcinoma in young white women, age 18 to 44 years. J Clin Oncol, 29, 1488-94.
Reimers, N., Kasper, H. U., Weissenborn, S. J., Stutzer, H., Preuss, S. F., Hoffinann, T. K., Speel, E. J., Dienes, H. P., Pfister, H. J., Guntinas-Lichius, O. & Klussmann, J. P. (2007). Combined analysis of HPV-DNA, p16 and EGFR expression to predict prognosis in oropharyngeal cancer. Int J Cancer, 120, 1731-8.
Ries L A G, Y. J., Keel G E, Eisner M P, Lin Y D, Horner M-J (editors). (2007). SEER Survival Monograph: Cancer Survival Among Adults: U. S. SEER Program, 1988-2001, Patient and Tumor Characteristics. National Cancer Institute, SEER Program, NIH Pub. No. 07-6215, Bethesda, Md., 2007., No. 07-6215.
Salvesen, H. B., Das, S. & Akslen, L. A. (2000). Loss of nuclear p16 protein expression is not associated with promoter methylation but defines a subgroup of aggressive endometrial carcinomas with poor prognosis. Clin Cancer Res, 6, 153-9.
Schache, A. G., Liloglou, T., Risk, J. M., Filia, A., Jones, T. M., Sheard, J., Woolgar, J. A., Helliwell, T. R., Triantafyllou, A., Robinson, M., Sloan, P., Harvey-Woodworth, C., Sisson, D. & Shaw, R. J. (2011). Evaluation of human papilloma virus diagnostic testing in oropharyngeal squamous cell carcinoma: sensitivity, specificity, and prognostic discrimination. Clin Cancer Res, 17, 6262-71.
Schantz, S. P. & Yu, G. P. (2002). Head and neck cancer incidence trends in young Americans, 1973-1997, with a special analysis for tongue cancer. Arch Otolaryngol Head Neck Surg, 128, 268-74.
Shiboski, C. H., Schmidt, B. L. & Jordan, R. C. (2005). Tongue and tonsil carcinoma: increasing trends in the U. S. population ages 20-44 years. Cancer, 103, 1843-9.
Shroyer, K. R. & Greer, R. O., Jr. (1991). Detection of human papillomavirus DNA by in situ DNA hybridization and polymerase chain reaction in premalignant and malignant oral lesions. Oral Surg Oral Med Oral Pathol, 71, 708-13.
Stevens, T. M., Caughron, S. K., Dunn, S. T., Knezetic, J. & Gatalica, Z. (2011). Detection of High-Risk HPV in Head and Neck Squamous Cell Carcinomas: Comparison of Chromogenic In Situ Hybridization and a Reverse Line Blot Method. Appl Immunohistochem Mol Morphol.
Straume, O., Sviland, L. & Akslen, L. A. (2000). Loss of nuclear p16 protein expression correlates with increased tumor cell proliferation (Ki-67) and poor prognosis in patients with vertical growth phase melanoma. Clin Cancer Res, 6, 1845-53.
Termine, N., Panzarella, V., Falaschini, S., Russo, A., Matranga, D., Lo Muzio, L. & Campisi, G. (2008). HPV in oral squamous cell carcinoma vs head and neck squamous cell carcinoma biopsies: a meta-analysis (1988-2007). Ann Oncol, 19, 1681-90.
Wiest, T., Schwarz, E., Enders, C., Flechtenmacher, C. & Bosch, F. X. (2002). Involvement of intact HPV16 E6/E7 gene expression in head and neck cancers with unaltered p53 status and perturbed pRb cell cycle control. Oncogene, 21, 1510-7.

### 6.3. Molecular Subtypes in Squamous Cell Carcinoma of the Head and Neck Cancer Reveal Exhibit Distinct Patterns of Chromosomal Gain and Loss of Canonical Cancer Genes, Including CCND1, CDKN2A, and EGFR

Here we describe the results of an integrated genomic analysis of 183 HNSCC tumor samples, making this one of the largest HNSCC studies to date. Gene expression (GE), DNA copy number (CN), or clinical data was available for all subjects. Multiple GE subtypes were detected, and the resulting expression patterns are similar to those previously found in HNSCC (8) and lung squamous cell carcinoma (LSCC) (7). All of the GE subtypes were also detected in head and neck cancer cell lines. In addition, we show that some CN gain and loss events are common to all subtypes, while others are found only in specific subtypes; that a number of these genomic events affect known oncogenes and tumor suppressors; and that these expression patterns and genomic events have clinical relevance.

Results

Unsupervised Discovery of HNSCC Expression Subtypes

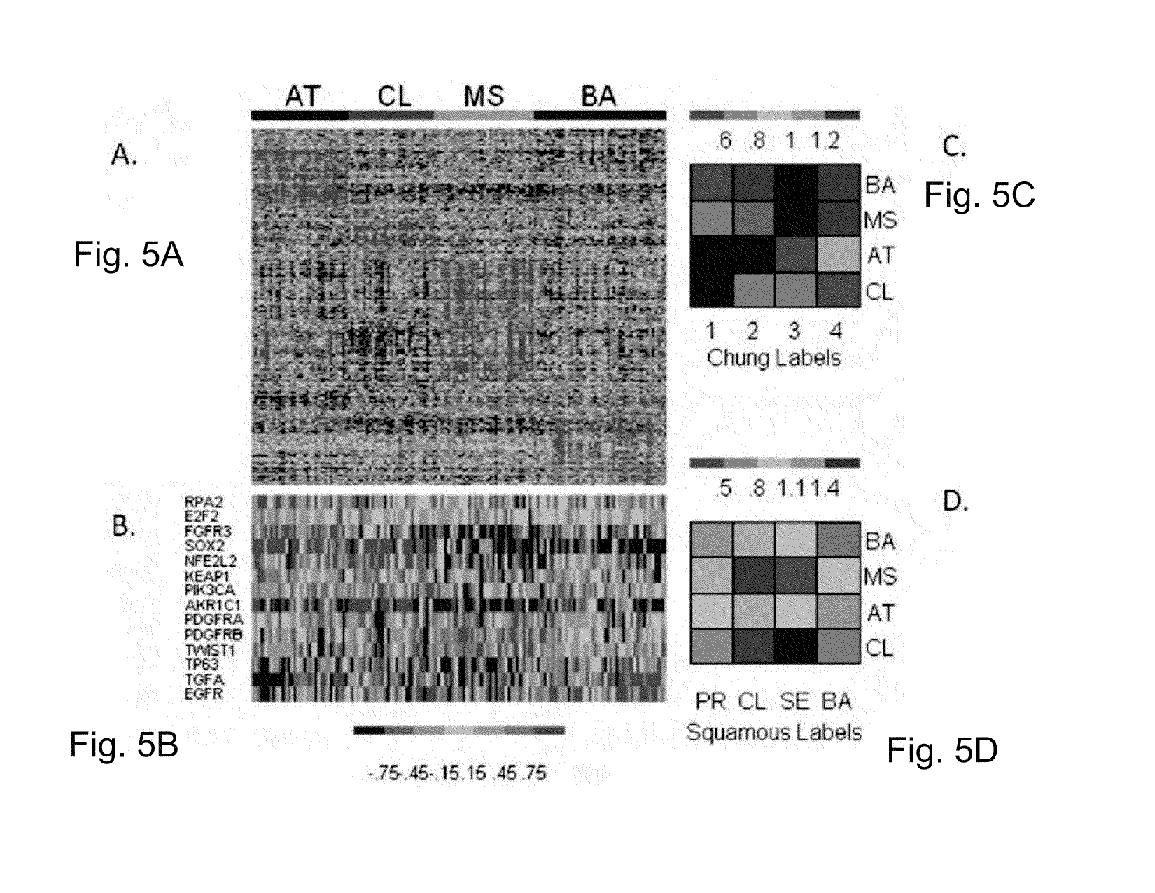

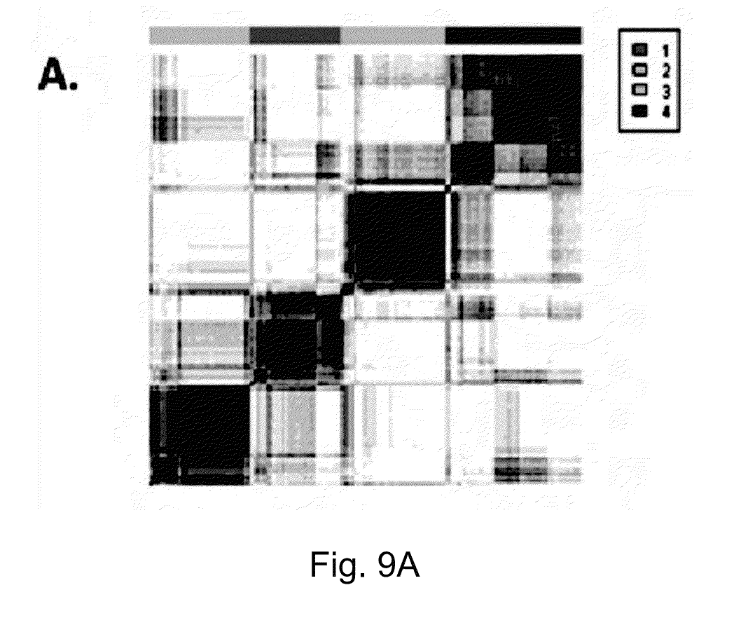

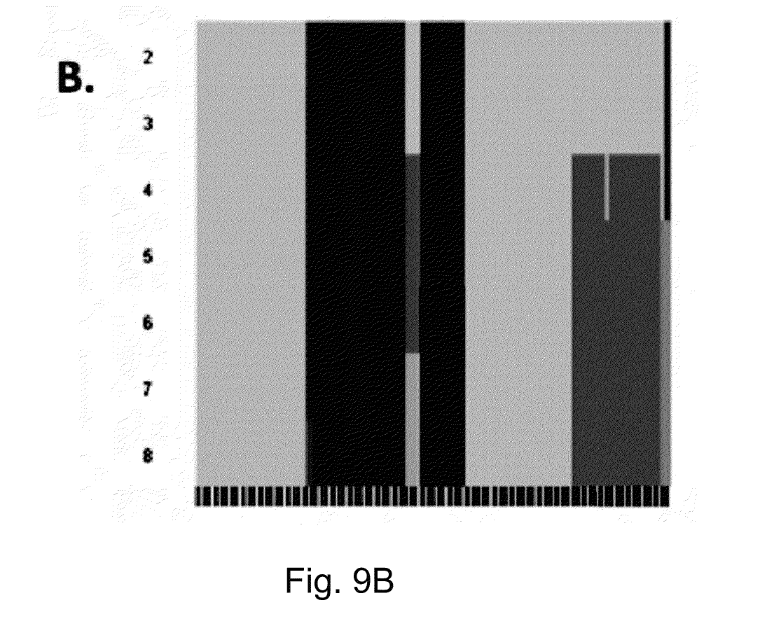

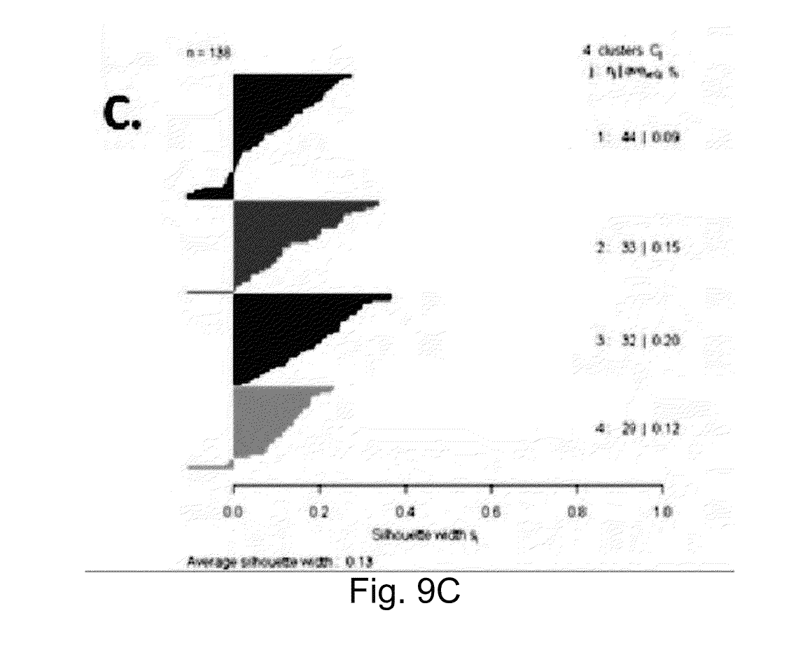

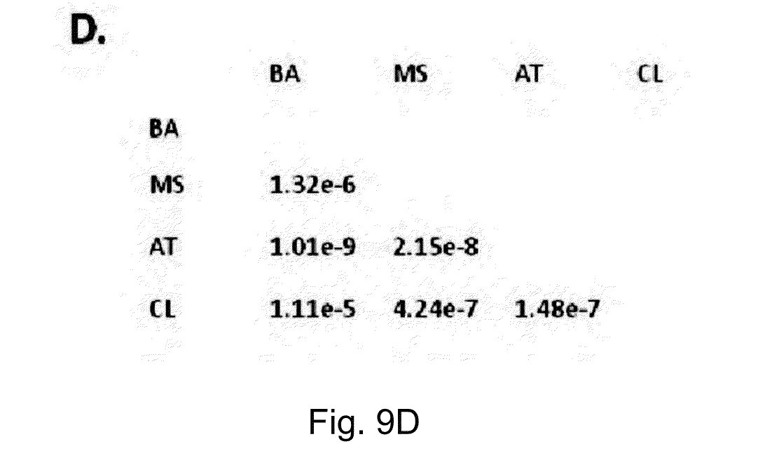

In order to address the question of whether statistically significant molecular subtypes can be elicited in HNSCC, we performed hierarchical clustering in an unsupervised and unbiased manner using well-established and objective techniques (7). As in the prior work by Chung, we document the presence of four gene expression clusters. Plots produced by ConsensusClusterPlus (9) (see FIGS. 9A and 9B) do not support the presence of additional statistically significant clusters in this dataset. To confirm the statistical significance of four clusters, SigClust (10) was applied using an unbiased set of the 2500 most variable genes across the cohort. All pairwise comparisons of the subtypes were examined using 1000 simulated samples and the original covariance estimation method. The SigClust p-values for all of the pairwise comparisons were significant at the 0.05 level after applying a Bonferroni correction for multiple comparisons (FIG. 9D). We refer to the expression subtypes as basal (BA), mesenchymal (MS), atypical (AT), and classical (CL) based on biological characteristics that are discussed below. A representative set of genes known or suspected to be relevant for head and neck cancer is shown (FIG. 5B), and test statistics for the association of all genes in the dataset with tumor subtype are provided in Tables 9-12.

Clinical Characteristics

The clinical characteristics of the patients included in the current study represent a broad cross section of patients with HNSCC that is highly representative of the population seen in a typical clinical practice (Table 5). There is no correlation of tumor subtype with age, gender, alcohol use, pack years, or tumor size. Tumor subtypes were statistically associated with site, although all sites had tumors in each of the expression subtypes, with one exception (hypopharynx showed no BA). Additionally, no site contributed more than 58% of its samples to one expression subtype. No expression subtype was made up of more than 68% of tumors from a single site. Therefore, unlike other molecular markers such as HPV or p16, we conclude that expression subtypes capture a dimension of biology which is not limited to a single anatomic site (11). There were additional statistically significant associations between tumor subtype and HPV status, treatment, node status, and overall stage. While not statistically significant, it is notable that more BA trended towards being well differentiated, whereas 13 of 16 poorly differentiated tumors were either MS or CL.

TABLE 5
Clinical Data. Summaries of select clinical
covariates in the HNSCC expression subtypes.

| | Total | Basal | Mesenchymal | Atypical | Classical | p-Value |
|---|---|---|---|---|---|---|
| Num. Patients | 138 | 44 | 33 | 32 | 29 | |
| Age (Years) | | | | | | 0.75 |
| Median | 57 | 60 | 57 | 56.5 | 58 | |
| Num., 40 | 9 | 5 | 3 | 1 | 0 | |
| Sex | | | | | | 0.64 |
| Female | 43 | 14 | 13 | 8 | 8 | |
| Male | 95 | 30 | 20 | 24 | 21 | |
| Race | | | | | | 0.34 |
| Black | 32 | 8 | 8 | 6 | 10 | |
| White | 104 | 36 | 24 | 26 | 18 | |
| Alcohol Use | | | | | | 0.44 |
| None/Light | 86 | 26 | 24 | 20 | 16 | |
| Heavy | 50 | 18 | 8 | 12 | 12 | |
| Smoking | | | | | | 0.11 |
| Never/Light | 27 | 13 | 6 | 6 | 2 | |
| Current/Former | 109 | 30 | 26 | 26 | 27 | |
| Mean (Packyears) | 36 | 36.7 | 33.1 | 30.1 | 45 | 0.13 |
| Differentiation | | | | | | 0.1 |
| Well | 26 | 14 | 5 | 3 | 4 | |
| Moderate | 92 | 27 | 21 | 25 | 19 | |
| Poor | 19 | 3 | 7 | 3 | 6 | |
| Tumor Site | | | | | | 1e−4* |
| Larynx | 30 | 10 | 4 | 5 | 11 | |
| Oral Cavity | 55 | 30 | 18 | 2 | 5 | |
| Oropharynx | 34 | 3 | 5 | 20 | 6 | |
| Hypopharynx | 13 | 0 | 2 | 5 | 6 | |
| Stage** | | | | | | .034* |
| I | 10 | 2 | 4 | 0 | 4 | |
| II | 14 | 8 | 1 | 2 | 3 | |
| III | 28 | 8 | 8 | 4 | 8 | |
| IVa | 77 | 26 | 16 | 22 | 13 | |
| IVb | 6 | 0 | 3 | 3 | 0 | |
| IVc | 10 | 0 | 0 | 1 | 0 | |
| Tumor Status | | | | | | 0.76 |
| T0-T2 | 40 | 12 | 10 | 8 | 10 | |
| T3-T4 | 77 | 30 | 16 | 16 | 15 | |
| Node Status | | | | | | 0.0026 |
| N0-N1 | 66 | 30 | 14 | 6 | 16 | |
| N2-N3 | 51 | 12 | 12 | 18 | 9 | |
| Treatment | | | | | | 4.50E−06 |
| Primary Chemo/RT | 62 | 11 | 13 | 26 | 12 | |
| Surgery | 74 | 33 | 20 | 5 | 16 | |
| HPV Status | | | | | | 0.035 |
| Negative | 82 | 27 | 21 | 17 | 17 | |
| Positive | 14 | 1 | 3 | 8 | 2 | |
| Chromosomal | 0.056 | 0.052 | 0.048 | 0.036 | 0.136 | 2.20E−04 |
| Instability Index | | | | | | |

Validation of Subtypes

We then turned our attention to the question as to whether the unbiased clusters detected in the current dataset corresponded to those previously reported by Chung et al. Using techniques for cluster validation developed previously (7) and described more fully in the Methods, we compared the centroids for each of the expression subtypes in the present study to the centroids for the subtypes of Chung et al. A clear correspondence was observed (FIG. 5C), with BA, MS, AT, and CL demonstrating the same expression patterns as the previous Chung classes 1, 2, 3, and 4, respectively. Having discovered four classes using independent and unbiased datasets and methods, we consider these four expression subtypes to be validated.

It is well known that squamous cell carcinomas from different sites in the body share some but not all molecular characteristics, such as deletion of chromosome 3p and amplification of chromosome 3q (12, 13). Based on our recently reported data on LSCC expression subtypes (7), we hypothesized that a correspondence to head and neck cancer might be observed. To investigate a broader phenotype of squamous cell carcinomas of the upper aerodigestive track, we extended the centroid predictor methodology and evaluated the correspondence of centroids from LSCC and HNSCC (FIG. 5D). Remarkably, a clear pattern of correlation was observed in which the BA, MS, and CL subtypes of HNSCC corresponded to the basal, secretory, and classical subtypes, respectively, of Wilkerson et al.

Affected Genes Suggest Distinct Biological Processes in Expression Subtypes

The fact that the subtypes exhibit different gene expression patterns suggests that each subtype has distinct biological characteristics. In an effort to clarify these properties we examine specific genes that are highly expressed in each class but not the others.

The basal phenotype, which was originally and perhaps best described in breast cancer (5), is seen in other epithelial cancers, notably LSCC (7, 14). A number of the basal signature genes found by Perou et al. (5) are highly expressed in BA, including CDH3, LAMA3, and COL17A1. Several other genes that are highly expressed in BA are important, including the transcription factor TP63, which we discuss in the following section. In addition, the DAVID (15) results indicate that the KEGG ErbB Signaling Pathway is enriched for genes that are highly expressed in BA, including TGFA, EGFR, MAPK1, and MAP2K1.

Kalluri and Weinberg (16) describe three biological settings in which cells undergo the epithelial-to-mesenchymal transition (EMT), two of which are cancer progression/metastasis and organ fibrosis. These authors indicate that mouse and cell culture studies of cancer cells with the mesenchymal phenotype exhibit high expression of ACTA2, VIM, DES, and TWIST, all of which are seen in MS. HGF, a growth factor that contributes to EMT and HNSCC progression (17), is also highly expressed in MS. Organ fibrosis occurs in various epithelial tissues, and is driven by the release of inflammatory signals and components of the extracellular matrix. Our DAVID analysis shows that the Focal Adhesion KEGG Pathway is over-represented by genes that are highly expressed in MS, including PDGFRA/B, as well as several laminins and collagen subunits.

It is known that EGFR expression is nearly universal in HNSCC (18), but recently unconfirmed reports have emerged that suggest an interaction between HPV+ tumors of the oropharynx and low EGFR expression (19). We observe low EGFR expression in AT, which represents a considerably broader range of tumors that is not limited by HPV status or tumor site. Kumar et al. (19) also find that CDKN2A and EGFR expression are negatively correlated, and we note that CDKN2A is highly expressed in AT when compared to all other classes. Other genes highly expressed in AT include RPA2, LIG1, and E2F2, all of which were found to be more highly expressed in HPV+ tumors than HPV− tumors by Slebos et al. (20). The DAVID results show enrichment for genes in the Fatty Acid Metabolism KEGG pathway, which includes a number of aldehyde dehydrogenase (ALDH) genes that are highly expressed in AT, such as ALDH3A1 and ALDH9A1. This is noteworthy because Muzio et al. (21) indicate that increased levels of these genes and other ALDHs have been seen in normal and cancer stem cells.

Studies in LSCC and normal airway epithelial cells have detected gene expression patterns associated with exposure to cigarette smoke (7, 22, 23). Our DAVID analysis indicates that the Xenobiotic Metabolism KEGG Pathway contains a number of genes that are highly expressed in CL. Among these are AKR1C1, AKR1C3, and GPX2, all of which are associated with smoking and oxidative stress (22, 23). These findings are striking in light of the fact that the heaviest smokers in our cohort are found in CL, a phenotype which has a clear correlate in the similarly-named subtype of LSCC. Additionally, a recent comprehensive investigation of LSCC found that KEAP1 and NFE2L2 are highly expressed in the classical subtype (14). Similar expression patterns are found in CL, which is compelling in light of the fact that NFE2L2 is a transcription factor that regulates genes involved in xenobiotic detoxification. High expression levels and increased copy number of PIK3CA are seen in CL, and previous studies (24, 25) have found associations between PIK3CA copy number gains and smoking status.

DNA Copy Analysis by Subtype

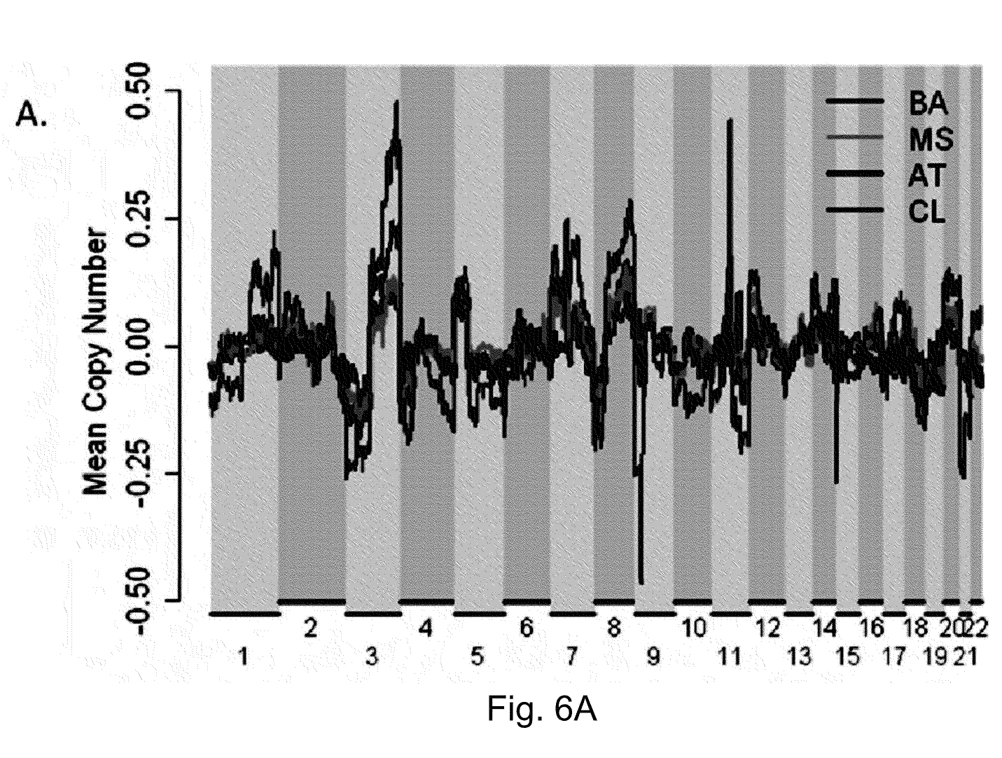

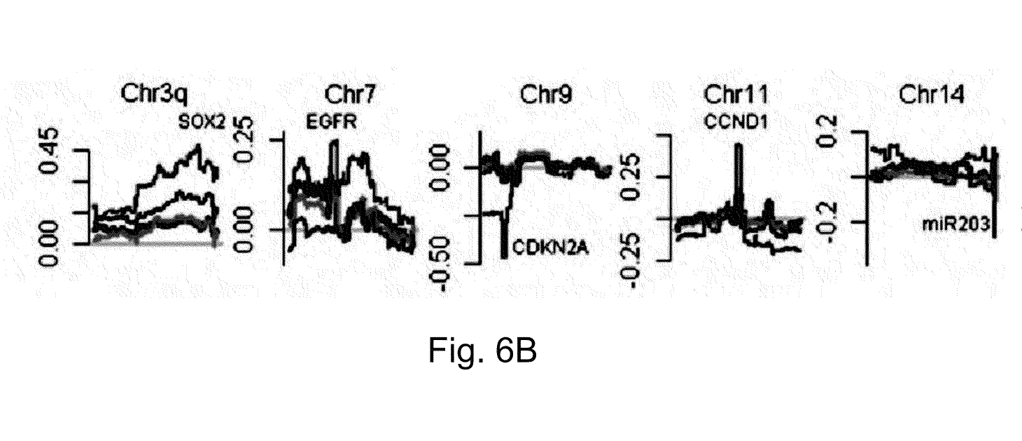

Having established the statistically significant nature of the HNSCC tumor subtypes and their correlation to similar subtypes in lung cancer and known cancer genes, we turned our attention to genomic alterations that might partially explain the subtype origins. To investigate differences in chromosomal abnormalities as potential sources of differential gene expression we generated plots of mean CN as a function of genomic position and tumor subtype (FIG. 6). As has been seen in other tumors, there are both concordant and distinct patterns of copy number alterations in key regions of the genome as a function of tumor subtype. In support of a common identity for this set of tumors, the most striking observation is a statistically significant shared alteration of chromosome 3 in all subtypes, including deletions of chr3p and the presence of a broad amplicon in chr3q that contains focal, high-level gains of PIK3CA and SOX2, and TP63 in some subtypes. By contrast, there are distinct differences in the canonical HNSCC chromosome 7p amplification. Statistically significant gains are also found overall in a broad region of chr7p that contains EGFR, and these are seen in BA, MS, and CL but completely absent in AT.

In addition to broad genomic events, there are striking focal events, some of which are shared, others of which are subtype specific. The well-known focal amplification in chr11q13.3, which contains CCND1, among other genes, is observed across all subtypes. Unexpectedly, a second focal amplification is observed in chr11q22 for BA only. This event is found in multiple samples even though it does not achieve statistical significance. The locus has been reported previously by Imoto et al. (26) in a study of esophageal squamous cell carcinoma that detected copy number gains in chr11q22-23, which contains cIAP1/BIRC2.

Overall, the most significant copy number losses are found in chromosomes 3p, 9p, and 14q. Statistically significant losses of chr3p are found overall and in each of the expression subtypes, but statistically significant losses of chr9p are found in BA and CL only. Losses of CDKN2A are seen in both subtypes, but BA exhibits hemizygous deletions over a broad region of chr9p, whereas CL has focal homozygous deletions. Focal loss is seen in chr14q32.33 for MS, AT, and CL, and these are the most significant losses for MS and AT. This region contains miR203, which is notable because it targets ΔNp63 (27).

Integration of Copy Number Changes and Differential Expression of Canonical HNSCC Genes by Expression Subtype

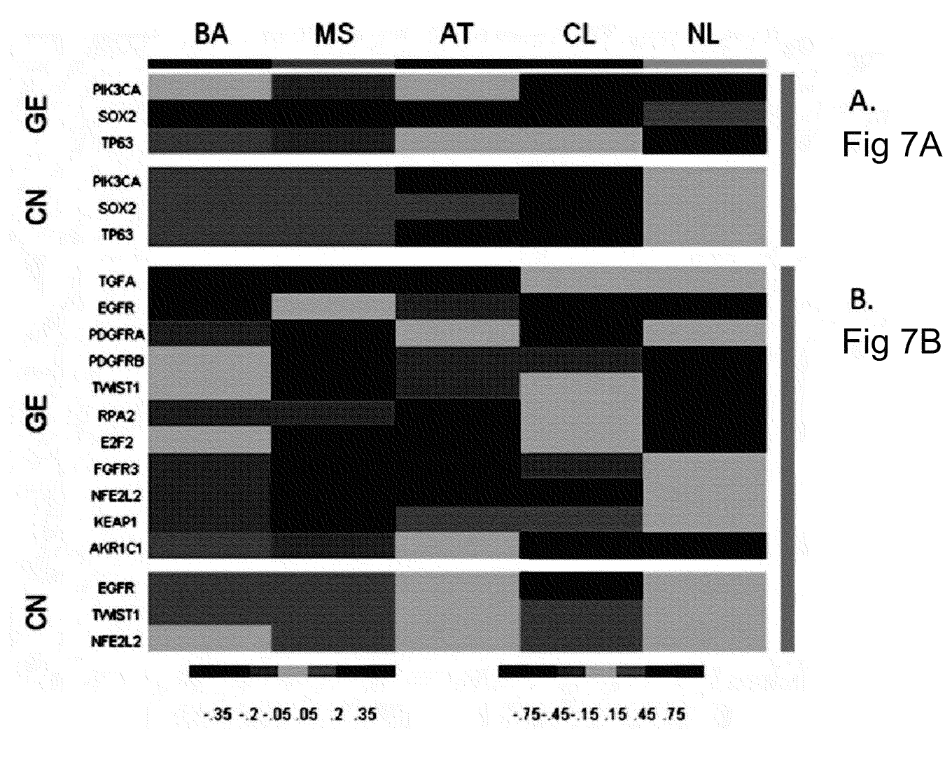

Having identified regions both concordant and discordant in copy number by expression subtype, we then considered whether expression of genes in those regions demonstrated changes that agree with the underlying copy number alterations (FIG. 7A). One of the quintessential genomic alterations associated with squamous cell carcinomas is amplification of chr3q. Unexpectedly, while all subtypes demonstrate amplification of chr3q, there was a distinct differential proportional usage of the three genes typically discussed as the targets of the amplicon: TP63, PIK3CA, and SOX2. The CL and AT subtypes demonstrate proportionally higher expression of SOX2 relative to MS and BA, which in fact appear to express less SOX2 than normal tonsil controls. By contrast, the BA subtype appears to express dramatically higher levels of TP63 than any other group. Similarly, although the MS subtype exhibits the chr3q amplicon, none of the putative target genes appear to be expressed at levels higher than normal tonsil. In sum, we conclude that this observation raises the possibility that the heterogeneity of HNSCC might in part be explained by differential usage of the transcription factors (SOX2 and TP63) and oncogene (PIK3CA) in the chr3q amplicon, which is more complex than has been previously reported (28). Consideration of the EGFR locus on chromosome 7 suggests, similarly, that EGFR may be more consistently targeted by some subtypes than others (FIG. 7B). These observations lend support to the possibility that differential usage of transcription factors and oncogenes, promoted in part by distinct copy number alterations, may contribute to the gene expression signatures that define the expression subtypes.

Focal Copy Number Events Involving Canonical Cancer Genes

In the preceding section we noted that the expression subtypes exhibit distinct patterns of copy number gain and loss. Now we focus our attention on genes known to play a role in HNSCC—CCND1, CDKN2A, and EGFR—and we consider copy number values at the specific gene loci, not the broader regions discussed above. Table 6 shows that copy number events at these genes are significantly associated with tumor subtype or approach significance, as exemplified by the fact that CCND1 focal amplification was present in 63% of CL samples while being distinctly uncommon in AT (16%). Similar results are seen for focal EGFR amplifications—the frequency of gains range from 0% (AT) to 31% (CL)—and CDKN2A losses—the frequency of losses range from 10% (MS) to 63% (CL).

Past studies have detected associations between distinct genomic events, and these findings provided insight into either the underlying biology or the clinical management of cancer patients (Zhu, Xing). In HNSCC, simultaneous CCND1 gains and CDKN2A losses have been studied by Namazie et al. (29) and Okami et al. (30), with Namazie et al. detecting an association between these genomic events. We find that CCND1 CN gains are associated with CDNK2A losses across all subtypes (Table 7), and that the joint event is associated with the expression subtypes (Table 6), thereby confirming and extending the results of Namazie et al.

TABLE 6
Focal Copy Number Events. Summaries of focal copy number events
for specific genes in the HNSCC expression subtypes.

| | Total | Basal | Mesenchymal | Atypical | Classical | P |
|---|---|---|---|---|---|---|
| CCND1Gain | | | | | | |
| No | 53 | 17 | 14 | 16 | 6 | .013 |
| Yes | 29 | 9 | 7 | 3 | 10 | |
| CDNK2A Loss | | | | | | |
| No | 62 | 20 | 19 | 17 | 6 | |
| Yes | 20 | 6 | 2 | 2 | 10 | .001 |
| Joint CCND1/ | | | | | | |
| CDKN2A Event | | | | | | |
| No | 70 | 23 | 20 | 18 | 9 | .006 |
| Yes | 12 | 3 | 1 | 1 | 7 | |
| EGFR Gain | | | | | | |
| No | 70 | 22 | 18 | 19 | 11 | .060 |
| Yes | 12 | 4 | 3 | 0 | 5 | |

TABLE 7
Overall Association of CCND1 Gains and CDKN2A Losses. Two-by-
two table illustrating CCND1 gains and CDKN2A losses, together
with Fisher's Exact Test p-value.

| | No CCND1 Gain | CCND1 Gain | Total | P |
|---|---|---|---|---|
| No CDKN2A Loss | 58 | 21 | 79 | .019 |
| CDKN2A Loss | 13 | 15 | 28 | |
| Total | 71 | 36 | 107 | |

Clinical Outcomes by Expression Subtype and Focal Genomic Alterations

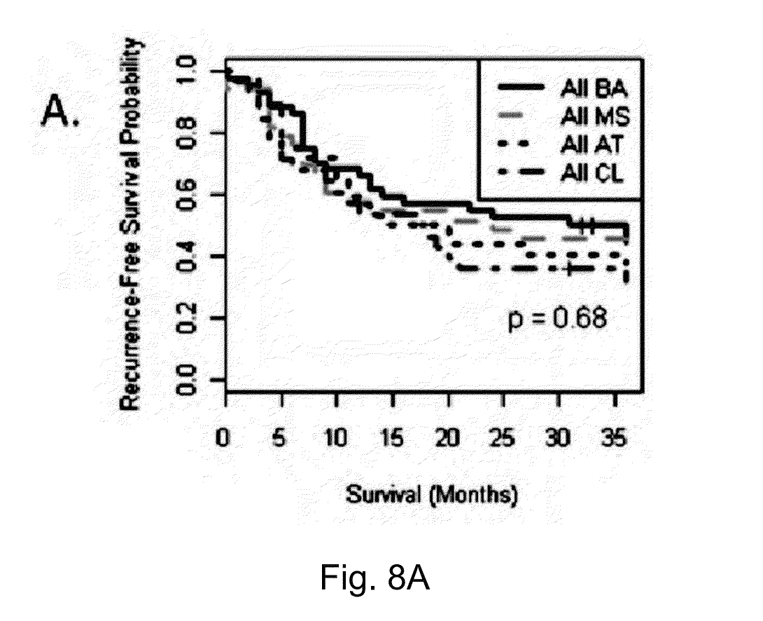

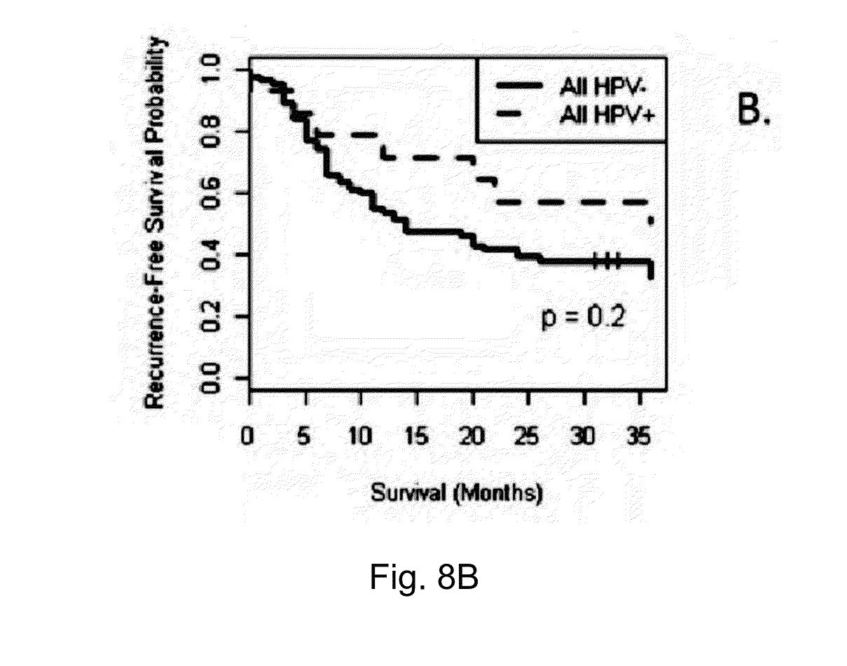

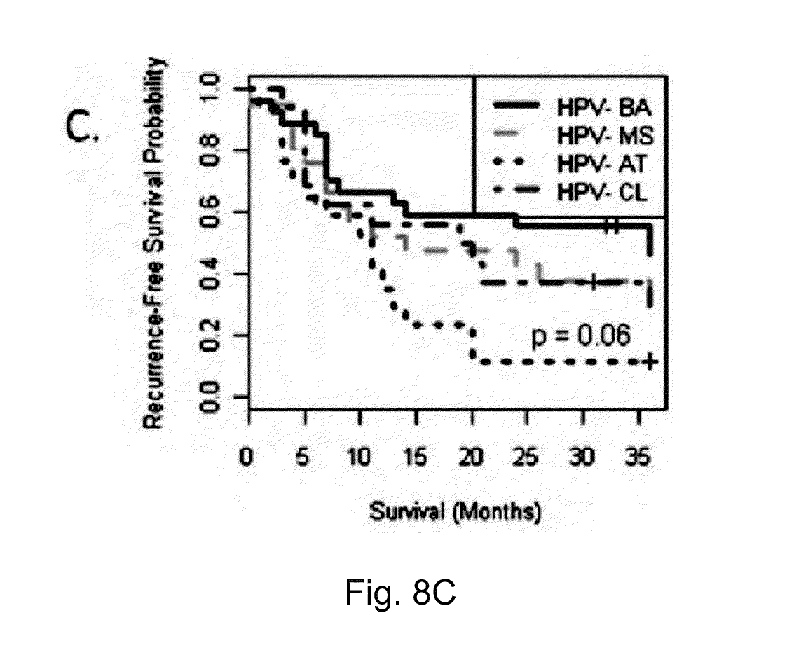

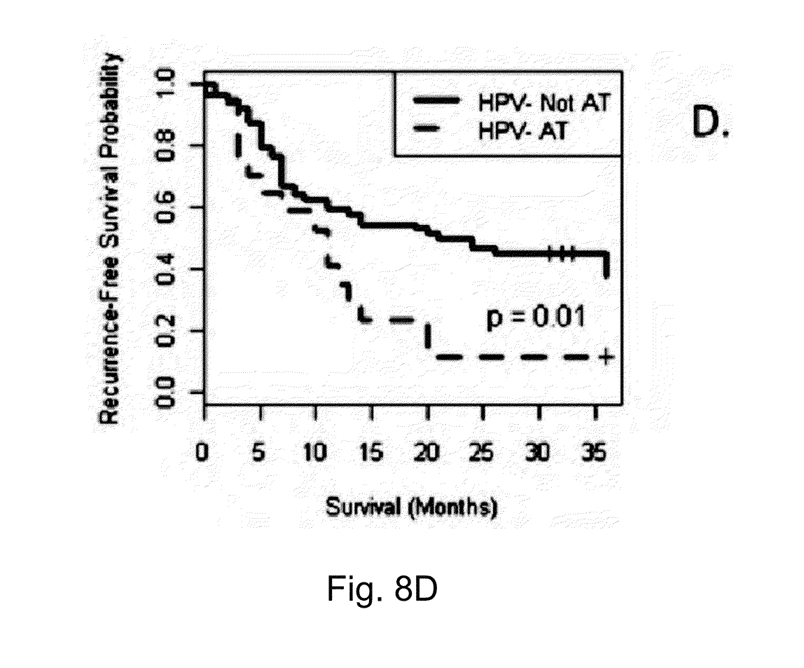

Having parsed the set of nearly 140 HNSCC tumors into expression subtypes, and in light of known risk factors such as HPV, we considered whether additional stratification for patient outcomes could be suggested. We first investigated whether the survival advantage reported by Chung et al. for their class 1 could be reproduced in the current cohort. We were unable to confirm this result, and in the current study there was no association between recurrence-free survival and tumor subtype, either overall (FIG. 8A) or when we restrict to late stage patients (not shown). These differences may be explained by the clinical heterogeneity of the disease combined with the fact that tumor site distributions in the two studies are markedly different.

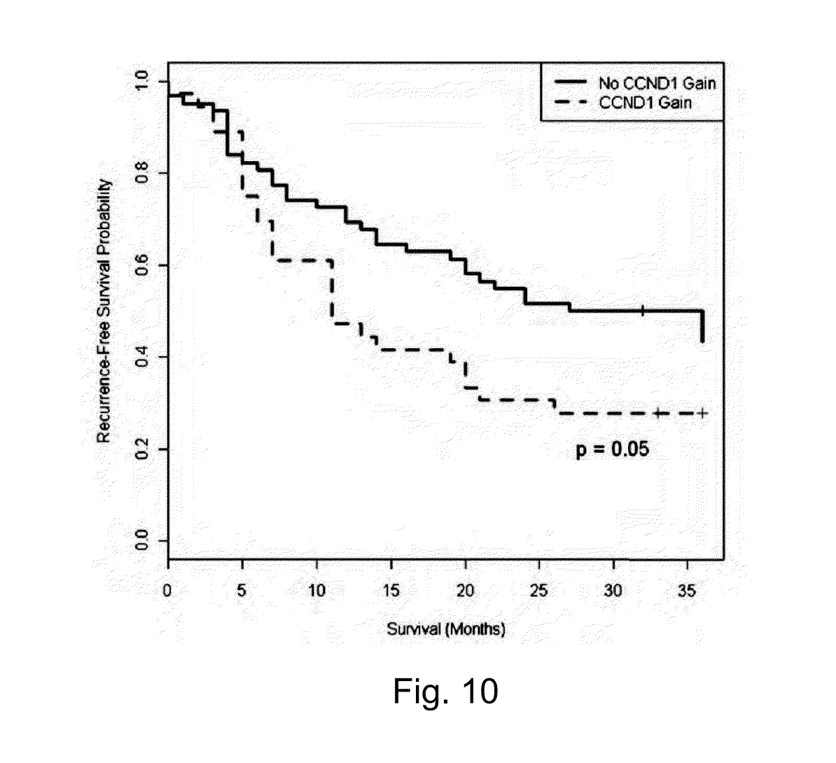

In order to clarify whether known or suspected confounders might affect our ability to detect subtype-specific differences in patient outcome, we evaluated the impact of HPV positivity on overall survival. We observed a relatively large but statistically insignificant effect due to the overall small number of patients. We therefore considered it reasonable to re-evaluate the cohort with HPV+ patients excluded. Exclusion of HPV+ patients revealed that the AT subgroup demonstrated a particularly unfavorable outcome (FIG. 8C), and this difference is statistically significant when compared to all other subtypes combined (FIG. 8D). We then accessed an independent set of tissue microarray (TMA) samples in an effort to validate this finding. It was not feasible to predict the tumor subtype of each TMA sample, so instead we used low EGFR and high p16 staining as a proxy for AT status. Although the difference in survival times is not statistically significant, when we restrict to TMA samples with negative HPV staining we obtain results that support the findings described above (FIG. 10).

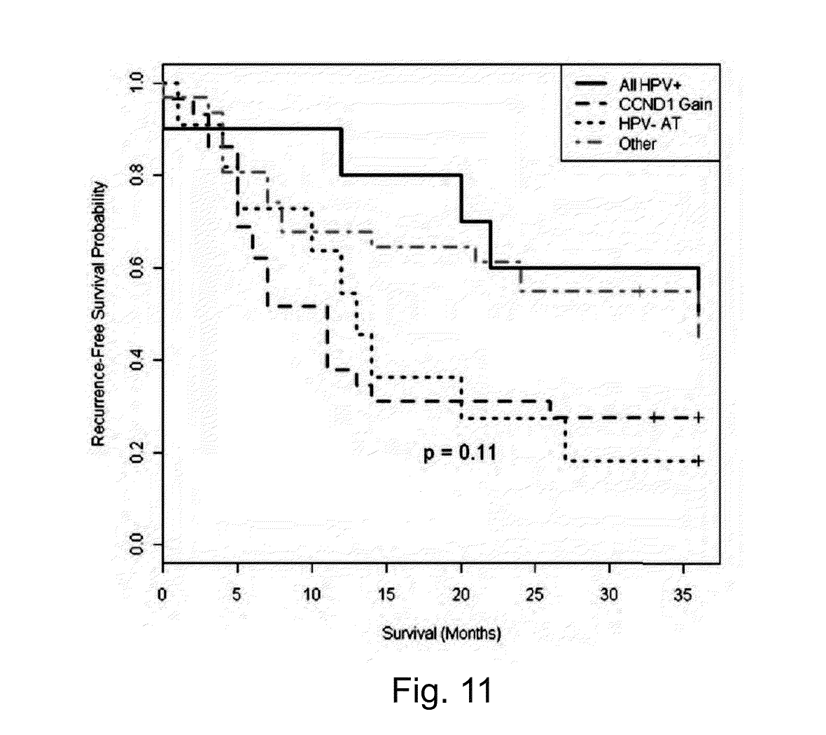

We also investigated whether any focal copy number events were associated with clinical outcome. Previous studies have detected a correlation between CCND1 gains and decreased recurrence-free survival times in HNSCC (31). We obtain similar findings when we examine the CN values for all tumor samples (FIG. 10), although our results are marginally significant (p=0.05). Remarkably few AT subjects exhibited CCND1 gains, and this suggests the presence of two largely distinct groups of patients with poor clinical outcomes: those with CCND1 amplifications and those that are HPV− and AT. FIG. 11 supports this conclusion.

Expression Subtypes in Model Systems

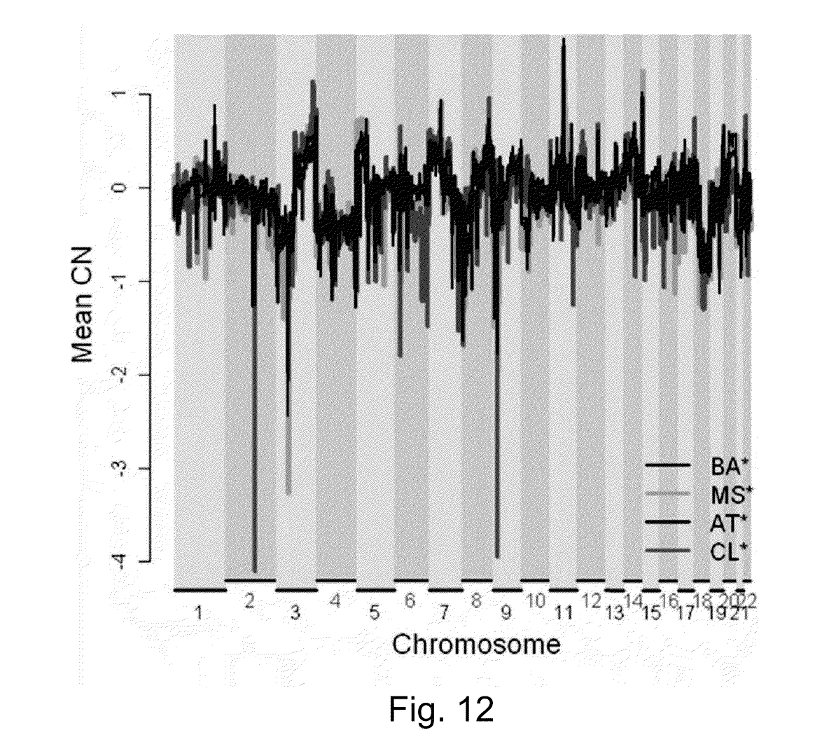

The Cancer Cell Line Encyclopedia (32) contains genomic data from over 900 human cancer cell lines, including both GE and CN data from 17 esophageal and 16 upper aerodigestive tract cell lines. We applied our centroid predictor to these cell lines and found that all four expression subtypes are present (Table 8). Summary plots of the CN values in each of the predicted subtypes show that many of the gain and loss events described earlier are also present in the cell lines (FIG. 12). These findings are particularly compelling in light of the clinical relevance of the expression subtypes because they provide the basis for future studies involving model systems.

TABLE 8
Predicted Expression Subtypes in HNSCC Cell Lines.

| | Predicted |
|---|---|
| Cell Line | Class |
| COLO680N_OESOPHAGUS | MS |
| KYSE140_OESOPHAGUS | CL |
| KYSE140_OESOPHAGUS | BA |
| KYSE180_OESOPHAGUS | CL |
| KYSE270_OESOPHAGUS | MS |
| KYSE30_OESOPHAGUS | AT |
| KYSE410_OESOPHAGUS | MS |
| KYSE450_OESOPHAGUS | CL |
| KYSE510_OESOPHAGUS | AT |
| KYSE520_OESOPHAGUS | MS |
| KYSE70_OESOPHAGUS | CL |
| OE19_OESOPHAGUS | AT |
| OE33_OESOPHAGUS | AT |
| TE11_OESOPHAGUS | CL |
| TE15_OESOPHAGUS | AT |
| TE1_OESOPHAGUS | MS |
| TE5_OESOPHAGUS | AT |
| TE9_OESOPHAGUS | AT |
| TT_OESOPHAGUS | CL |
| BICR31_UPPER_AERODIGESTIVE_TRACT | MS |
| CAL27_UPPER_AERODIGESTIVE_TRACT | BA |
| DETROIT562_UPPER_AERODIGESTIVE_TRACT | MS |
| FADU_UPPER_AERODIGESTIVE_TRACT | AT |
| HS840T_UPPER_AERODIGESTIVE_TRACT | MS |
| HSC2_UPPER_AERODIGESTIVE_TRACT | BA |
| HSC3_UPPER_AERODIGESTIVE_TRACT | BA |
| HSC4_UPPER_AERODIGESTIVE_TRACT | AT |
| PECAPJ15_UPPER_AERODIGESTIVE_TRACT | AT |
| PECAPJ34CLONEC12_UPPER_AERODIGESTIVE_TRACT | BA |
| PECAPJ41CLONED2_UPPER_AERODIGESTIVE_TRACT | MS |
| PECAPJ49_UPPER_AERODIGESTIVE_TRACT | MS |
| SCC15_UPPER_AERODIGESTIVE_TRACT | MS |
| SCC25_UPPER_AERODIGESTIVE_TRACT | MS |
| SCC4_UPPER_AERODIGESTIVE_TRACT | MS |
| SCC9_UPPER_AERODIGESTIVE_TRACT | BA |
| SNU1076_UPPER_AERODIGESTIVE_TRACT | AT |
| SNU899_UPPER_AERODIGESTIVE_TRACT | AT |

### DISCUSSION

Our primary results are that four gene expression subtypes exist in HNSCC—basal, mesenchymal, atypical, and classical—and that these subtypes exhibit distinct patterns of chromosomal gain and loss. We also show that these subtypes have biological and clinical relevance, and therefore that they provide a useful and informative method of classifying HNSCC tumors that complements existing methods based on histology and tumor site. Analysis of publicly available expression datasets reveals that these subtypes are reproducible in HNSCC (8) and are similar to those found in LSCC (7). All of the expression subtypes were detected in HNSCC cell lines, a finding that may provide the basis for future studies.

The expression patterns found in the subtypes suggests the presence of fundamental differences in the underlying biology of the associated tumors. Gene expression in BA shows a strong similarity to the signature found in basal cells from the human airway epithelium, including high expression of genes associated with the extracellular matrix (LAMA3, KRT17), receptors and ligands (EGFR, EREG), and transcription factors (TP63). Tumors in MS are exemplified by elevated expression of genes associated with EMT, including mesenchymal markers (VIM, DES), relevant transcription factors (TWIST1), and growth factors (HGF). In contrast to what is typically seen in HNSCC, tumors in AT exhibit no EGFR gains, as well as few gains of CCND1 or losses of CDKN2A. AT tumors also have a strong HPV+ signature, as evidenced by elevated expression of CDKN2A, RPA2, and E2F2. Tumors in CL show high expression of genes associated with exposure to cigarette smoke, including AKR1C1/3 and GPX2, and also have the heaviest smoking histories. CN gains and losses in the CL subtype tend to have greater magnitude when compared to what is found in the other subtypes, which reflects the increased level of chromosomal instability present in this class (Table 5).

The differences in the expression patterns found in the subtypes are clinically relevant. TP63 produces six distinct proteins—TAp63α/β/γ and ΔNp63α/β/γ —and ΔNp63 is the most abundant isoform in HNSCC (33). Yang et al. (33) show that ΔNp63 promotes cell proliferation, in part through its interactions with NF-κB proteins Re1A and cRel. Chatterjee et al. (34) noted that exposure to cisplatin led to decreased levels of ΔNp63, so this treatment may be particularly effective for patients in BA. Barbieri et al. (35) showed that loss of TP63 in HNSCC cell lines led to the acquisition of a mesenchymal phenotype, which is compelling in light of the low expression levels of TP63 seen in MS. Martin and Cano (36) indicate the elevated expression of TWIST1 or BMI1 in HNSCC cell lines can increase the likelihood of invasiveness and migration. Because MS tumors exhibit an EMT phenotype and increased expression of both TWIST1 and BMI1, these subjects may be more likely to develop distant metastases. The fact that EGFR is overexpressed in the vast majority of HNSCC tumors makes EGFR inhibitors are an attractive treatment option for this disease. However, these therapies are less likely to be effective in AT tumors because EGFR expression is lower than in the other expression subtypes. SOX2 and ALDH1 are highly expressed in AT and CL, and both of these genes are putative cancer stem cell markers because of their contributions to self-renewal and a pleuripotent phenotype (37, 38). The protein product of PIK3CA is p110a, which phosphorylates Akt. Activated Akt contributes to the survival of tumor cells, and thus oncogenic transformation (39). West et al. (40) show that exposing normal lung epithelial cells to nicotine facilitates activation of Akt by making it dependent on PI3K alone. This observation, combined with the high levels of smoking seen in CL, suggests that PI3 kinase inhibitors provide an attractive treatment option for CL tumors.

There were several limitations to this study. First, we do not have GE, CN, and clinical data for all study subjects, which limits our ability to jointly analyze these variables. In part this was the result of the presence of a technical artifact that caused our quality control procedure to eliminate over 20% of the CN arrays. In addition, it is not clear which isoform(s) of TP63 is being assayed by our gene expression arrays, and unfortunately the role that TP63 plays in the basal subtype cannot be fully appreciated without knowledge of these isoforms. The incomplete nature of our HPV data is also problematic.

Materials and Methods

Tumor Collection and Genetic Assays

Frozen, surgically extracted, macrodissected head and neck tumors were collected at the University of North Carolina Hospital under Institutional Review Board protocol #01-1283. Tumor RNA was extracted and mRNA expression was assayed using Agilent 44K microarrays. Tumor DNA was extracted and DNA copy number was assayed using Affymetrix GenomeWide SNP 6.0 chips.

mRNA Expression Analysis

Quality control procedures were applied to microarray probe-level intensity files. A total of 138 tumor arrays remained after removing low-quality arrays, duplicate arrays, and arrays from non-HNSCC samples. The normexp background correction and loess normalization procedures (39) were applied to the probe-level data. After log transformation, probes were matched to a common gene database to produce expression values for 15597 genes.

Unsupervised Expression Subtype Discovery

The procedure described here is similar to that which appeared in Wilkerson et al. After expression values were gene median centered, gene variability was computed using the median absolution deviation. The 2500 most variable genes were selected. ConsensusClusterPlus (9) was used to perform unsupervised clustering for these genes in the 138 arrays, and henceforth we refer to the resulting class labels as the “UNC classes.” This procedure was performed with 1000 randomly selected sets of microarray samples using a sampling proportion of 80% and a distance metric equal to one minus the Pearson correlation coefficient.

Differentially Expressed Genes and Metabolic Pathways

Differentially expressed genes were detected with the R package samr (42) using an FDR threshold of 0.01. For each of the UNC classes we compared the gene expression values in the class to all other classes combined. DAVID (15) was then used to find KEGG pathways that show enrichment for the highly expressed genes in each class. In addition, differentially expressed genes with known functional categories, e.g. transcription factors, were found by comparing the class-specific gene lists to known gene ontology categories (43).

Published Expression Data

The microarray probe-level intensity files produced by Chung et al. were subjected to background correction, normalization, and gene-level summarization procedures similar to those described above. This produced gene expression values for 60 subjects and 8224 genes. The class labels for these 60 arrays that appeared in (7) are referred to as the “Chung classes.”

Validation of Expression Subtypes

Consensus clustering assigns a class label to every array. As a result, some arrays may not be representative of their class. Using silhouette widths (44), we identified a set of 125 “core” samples whose expression patterns are more similar to those of members of their own subtype than other subtypes. C1aNC (45), a classification method based on nearest centroids, was then applied to the UNC expression data from the core samples in an effort to create a set of classifier genes whose expression signature could be used to classify new samples. Minimizing the cross-validation error rate produced a list of 840 classifier genes (210 genes per class).

We identified the classifier genes whose expression values are also present in the Chung expression dataset, and then restricted the UNC and Chung expression datasets to these genes. After gene median centering each dataset separately, we found the centroid for each of the UNC and Chung classes by computing the median expression value for each gene over all arrays having the appropriate class label. As in (6), the distances between the UNC and Chung centroids were computed using the metric one minus the Pearson correlation coefficient. This validation process was repeated using the LSCC data of Wilkerson et al.

DNA Copy Number Analysis

CEL files were subjected to quality control procedures using the Affymetrix Genotyping Console, and arrays that produced contrast QC measurements above the default threshold of 0.4 were removed from subsequent analyses. The intensity values in the CEL files were then converted to log_{—}2 copy number values using the R package aroma (46) and a pooled collection of normal samples. A total of 107 arrays remained after manually reviewing the genome-wide copy number profiles, 82 of which have expression class labels. Missing values were imputed using the non-missing value from the closest probe. Segmentation was performed using DNAcopy (47). Recurrent copy number gains and losses were detected with DiNAMIC (48) after smoothing and median centering the copy number profiles, as was done in (49). Gains and losses are classified as statistically significant if the resulting DiNAMIC p-values are less than 0.05. Regions harboring recurrent CN gains and losses were found using the confidence interval procedure of Walter et al. (50) at level 0.95. This was performed for the collection of all 107 arrays, as well as after restricting to the arrays in each of the four UNC classes.

Copy Number Gains and Losses of Canonical Cancer Genes

The gene-specific copy number was determined by computing the mean of all segmented copy number values at probes lying within or immediately adjacent to the gene. For each subject we classify a gene as having a copy number gain (loss) if the gene-specific copy number is above 0.35 (below −0.35), which is approximately two standard deviations above (below) the mean of all segmented copy number values.

Statistical Analysis

R 2.12.2 was used to perform all data analysis. The statistical significance of associations between all categorical variables was assessed with Fisher's Exact Test or a Monte Carlo version of Fisher's Exact Test (p-values include an asterisk). Global F-tests were used to assess the statistical significance of associations of continuous variables with the expression subtypes. The survival package was used to perform all survival analyses. Recurrence-free survival (RFS) time was defined to be the time in months from surgery to death, recurrence, or loss to follow-up.

Chromosomal Instability Index

For a given subject, we compute the median of the absolute value of the smoothed, segmented copy number values in each chromosome arm. The median of the arm-specific medians is defined to be the chromosomal instability index, which is similar to the definition that appears in (49).

Cancer Cell Line Data

CN and GE data are available for 18 esophagus and 19 “upper aerodigestive tract” cell lines that are classified as squamous cell carcinoma in the CCLE. GE data in the cell lines is available for 803 of the 840 genes in our classifier. After restricting to these common genes, we normalized the GE data for the cell lines so that it had the same gene-specific means and standard deviations as in our classifier. We then used the centroid-based method described above to predict expression subtypes for the cell lines. See also Walter et al. 2013 PLOS ONE 8(2) e56823 (pub. online 2013 Feb. 22) the contents of which are hereby incorporated in their entity.

Tables 9-12 list gene signatures for the different head and cancer subtypes. See GeneCards (www.genecards.org), U.S. National Library of Medicine, National Center for Biotechnology Information (NCBI) Gene database (http://www.ncbi.nlm.nih.gov/sites/entrez?db=gene), or European Bioinformatics Institute (EBI) and the Wellcome Trust Sanger Institute (WTSI), Ensembl database (http://useast.ensembl.org/index.html) or BLAT on University of California Santa Cruz (UCSC) Genome Browser (http://genome.ucsc.edu/cgi-bin/hgBlat) for additional information such as sequences, single nucleotide polymorphisms (SNPs).

TABLE 9
Top 20 Gene signatures associated with the Basal, Mesenchymal,
Atypical, and Classical head and neck cancer subtypes

| GeneName | Basal | Mesenchymal | Atypical | Classical |
|---|---|---|---|---|
| ADAM23 | −0.15768498 | −0.28438884 | −0.03941938 | 1.63604138 |
| C21orf81 | −1.02178105 | −0.39055418 | 2.2240084 | −0.30592688 |
| CD74 | −0.42754269 | 0.86342545 | 0.21401578 | −1.63861677 |
| CYP26A1 | −0.57250721 | −0.07309238 | 0.42973868 | 2.20948577 |
| DSG1 | 1.8154619 | −0.80673989 | −0.74872168 | −0.73916802 |
| EPGN | 1.82052624 | −0.6381393 | −0.04295136 | −0.46785319 |
| FAM3B | −0.81447979 | −0.63995746 | 3.95042642 | −0.30066266 |
| FLRT3 | 1.4379561 | −0.00129233 | −1.65350203 | −0.75091586 |
| FNDC1 | −0.29837838 | 1.62151195 | −0.79040322 | −0.6913857 |
| HLA-DRA | −0.1586953 | 0.49384548 | −0.0019877 | −1.7012316 |
| INHBA | 0.43259054 | 0.73909525 | −2.82854664 | −0.2586106 |
| MAL | −0.13346434 | −0.9253673 | 4.44770445 | −0.42662375 |
| MICALCL | 1.80741144 | −0.11841642 | −0.94374877 | −0.48509442 |
| NNMT | −0.1886067 | 1.6402351 | −0.62415093 | −0.5797558 |
| PNLIPRP3 | 1.57187425 | −0.27236449 | −0.23471355 | −0.27185475 |
| PRAME | −0.69486884 | −0.42350375 | −0.55356105 | 1.405513 |
| RARRES2 | −0.70373832 | 1.68811559 | −0.00931006 | −0.44940697 |
| RGS16 | −0.33545592 | 1.25137566 | 0.01059367 | −0.05440816 |
| SFRP4 | −0.82161397 | 3.416405 | −0.6919744 | −0.47959283 |
| TMPRSS11B | 0.09453679 | −1.276955 | 4.01044768 | −0.66989218 |

TABLE 10
Top 40 Gene signatures associated with the Basal, Mesenchymal,
Atypical, and Classical head and neck cancer subtypes

| GeneName | Basal | Mesenchymal | Atypical | Classical |
|---|---|---|---|---|
| ADAM23 | −0.15768498 | −0.28438884 | −0.03941938 | 1.63604138 |
| AKR1C1 | 0.10955915 | −0.96795677 | 0.06966853 | 2.59033056 |
| ALDH1A1 | −1.14252432 | −0.26944278 | 2.43831265 | 2.81560864 |
| ATP6V0A4 | 0.03700116 | −0.40843006 | 1.90064795 | −0.15175878 |
| C21orf81 | −1.02178105 | −0.39055418 | 2.2240084 | −0.30592688 |
| CD74 | −0.42754269 | 0.86342545 | 0.21401578 | −1.63861677 |
| CT45A1 | −0.4542219 | 0.28208845 | −0.43551205 | 2.92245327 |
| CXCL12 | −0.94542857 | 2.14056825 | 0.25053663 | −1.24824181 |
| CYP26A1 | −0.57250721 | −0.07309238 | 0.42973868 | 2.20948577 |
| CYP2E1 | −0.22947631 | −0.27355637 | 1.82410689 | −0.24577868 |
| CYP4B1 | −0.5306774 | −0.41998758 | 2.66426944 | −1.0171877 |
| DSG1 | 1.8154619 | −0.80673989 | −0.74872168 | −0.73916802 |
| EPGN | 1.82052624 | −0.6381393 | −0.04295136 | −0.46785319 |
| EREG | 1.49106195 | −0.41934415 | −0.82872489 | −1.09958057 |
| FAM3B | −0.81447979 | −0.63995746 | 3.95042642 | −0.30066266 |
| FAM46B | 0.99484489 | −0.51747884 | 0.23970165 | −1.47275374 |
| FLRT3 | 1.4379561 | −0.00129233 | −1.65350203 | −0.75091586 |
| FNDC1 | −0.29837838 | 1.62151195 | −0.79040322 | −0.6913857 |
| HLA-DRA | −0.1586953 | 0.49384548 | −0.0019877 | −1.7012316 |
| HSPC159 | 1.19353826 | −0.81737074 | −0.37077017 | −0.63285687 |
| INHBA | 0.43259054 | 0.73909525 | −2.82854664 | −0.2586106 |
| KRT19 | −2.06146456 | −0.65158041 | 1.92393502 | 0.95873294 |
| MAL | −0.13346434 | −0.9253673 | 4.44770445 | −0.42662375 |
| MICALCL | 1.80741144 | −0.11841642 | −0.94374877 | −0.48509442 |
| NNMT | −0.1886067 | 1.6402351 | −0.62415093 | −0.5797558 |
| NTS | −0.67695536 | 0.11676859 | 0.83353897 | 2.50807266 |
| OLFML3 | −0.4126521 | 0.95819848 | −0.30628062 | −0.44250578 |
| PDGFRL | −0.44480374 | 1.19873582 | −0.16399951 | −0.1862348 |
| PLAC8 | −0.9620995 | 0.02425181 | 2.62687439 | −0.06422655 |
| PNLIPRP3 | 1.57187425 | −0.27236449 | −0.23471355 | −0.27185475 |
| PRAME | −0.69486884 | −0.42350375 | −0.55356105 | 1.405513 |
| RAB6B | −0.31507222 | 0.03054956 | −0.1395046 | 0.86236765 |
| RARRES2 | −0.70373832 | 1.68811559 | −0.00931006 | −0.44940697 |
| RGS16 | −0.33545592 | 1.25137566 | 0.01059367 | −0.05440816 |
| RGS20 | 0.98685367 | −0.12811884 | −1.29763847 | −0.37923251 |
| SFRP2 | −0.59354926 | 1.8237165 | −0.09747845 | −0.54071748 |
| SFRP4 | −0.82161397 | 3.416405 | −0.6919744 | −0.47959283 |
| TMPRSS11B | 0.09453679 | −1.276955 | 4.01044768 | −0.66989218 |
| TMPRSS2 | −0.70192603 | −0.44385887 | 1.80509878 | −0.22099086 |
| VCAN | −0.54948326 | 1.40546253 | −0.72685015 | 0.06185013 |

TABLE 11
Top 79 Gene signatures associated with the Basal, Mesenchymal,
Atypical, and Classical head and neck cancer subtypes

| GeneName | Basal | Mesenchymal | Atypical | Classical |
|---|---|---|---|---|
| ADAM23 | −0.15768498 | −0.28438884 | −0.03941938 | 1.63604138 |
| AEBP1 | −0.38230623 | 1.12002045 | −0.56598409 | −0.18350693 |
| AIF1 | −0.32842333 | 1.00544966 | 0.06343709 | −0.91262284 |
| AKR1C1 | 0.10955915 | −0.96795677 | 0.06966853 | 2.59033056 |
| ALDH1A1 | −1.14252432 | −0.26944278 | 2.43831265 | 2.81560864 |
| ANGPTL2 | −0.45249404 | 1.08646286 | −0.29539152 | −0.22380734 |
| ATP6V0A4 | 0.03700116 | −0.40843006 | 1.90064795 | −0.15175878 |
| C1orf54 | −0.29164441 | 0.92639232 | 0 | −0.36312862 |
| C21orf81 | −1.02178105 | −0.39055418 | 2.2240084 | −0.30592688 |
| C2orf54 | 0.10713098 | −1.45600938 | 1.25342756 | −1.41057974 |
| CABYR | −0.15315465 | −0.12156949 | 0.23834449 | 1.14630605 |
| CALB1 | 0.0232156 | −0.35916111 | −0.26291639 | 3.37488446 |
| CAND2 | −0.67367514 | 0.29651763 | 0.7447677 | 0.49467508 |
| CCL19 | −0.97716428 | 1.20781944 | 0.96728914 | −1.87543014 |
| CD74 | −0.42754269 | 0.86342545 | 0.21401578 | −1.63861677 |
| CLCA4 | −0.02356618 | −1.04558037 | 2.05425635 | −1.06207291 |
| CLDN10 | −0.59899409 | −0.19487387 | 2.57260031 | 0.26267966 |
| CT45A1 | −0.4542219 | 0.28208845 | −0.43551205 | 2.92245327 |
| CXCL12 | −0.94542857 | 2.14056825 | 0.25053663 | −1.24824181 |
| CYP26A1 | −0.57250721 | −0.07309238 | 0.42973868 | 2.20948577 |
| CYP2E1 | −0.22947631 | −0.27355637 | 1.82410689 | −0.24577868 |
| CYP4B1 | −0.5306774 | −0.41998758 | 2.66426944 | −1.0171877 |
| CYP4F11 | 0.14190333 | −0.52423233 | 0.30258293 | 2.20934753 |
| D4S234E | 1.18370891 | −1.24307146 | −0.30169237 | −0.33075613 |
| DSG1 | 1.8154619 | −0.80673989 | −0.74872168 | −0.73916802 |
| EPGN | 1.82052624 | −0.6381393 | −0.04295136 | −0.46785319 |
| EREG | 1.49106195 | −0.41934415 | −0.82872489 | −1.09958057 |
| FAM3B | −0.81447979 | −0.63995746 | 3.95042642 | −0.30066266 |
| FAM46B | 0.99484489 | −0.51747884 | 0.23970165 | −1.47275374 |
| FLRT3 | 1.4379561 | −0.00129233 | −1.65350203 | −0.75091586 |
| FNDC1 | −0.29837838 | 1.62151195 | −0.79040322 | −0.6913857 |
| FOXA1 | −0.83496453 | −0.72889492 | 1.92834742 | 0.29299191 |
| FUT6 | 0.08606845 | −0.42190621 | 1.3162495 | −0.31445622 |
| FUT7 | −0.07903759 | −0.15797373 | 1.06279338 | −0.27051573 |
| GPX2 | −0.58694075 | −0.82589032 | 0.80446085 | 1.97921624 |
| HLA-DMA | −0.21878457 | 0.69511371 | 0.4216724 | −1.16654857 |
| HLA-DPB1 | −0.37387631 | 0.59564051 | 0.1068635 | −1.14040262 |
| HLA-DRA | −0.1586953 | 0.49384548 | −0.0019877 | −1.7012316 |
| HSPC159 | 1.19353826 | −0.81737074 | −0.37077017 | −0.63285687 |
| INHBA | 0.43259054 | 0.73909525 | −2.82854664 | −0.2586106 |
| KLK5 | 0.71178548 | 0.07715915 | −1.3059174 | −1.45870068 |
| KLK7 | 1.10613942 | −0.96521669 | 0.04861242 | −1.45110867 |
| KRT19 | −2.06146456 | −0.65158041 | 1.92393502 | 0.95873294 |
| LRIG1 | −0.84805801 | 0.46565975 | 0.30989699 | 0.30327544 |
| MAL | −0.13346434 | −0.9253673 | 4.44770445 | −0.42662375 |
| MGP | −0.6042429 | 1.32936046 | 0.21361856 | −0.5990463 |
| MICALCL | 1.80741144 | −0.11841642 | −0.94374877 | −0.48509442 |
| MRAP2 | −0.44588194 | −0.13216983 | 0.34355464 | 1.23894695 |
| NID2 | −0.19358131 | 0.91236645 | −0.81283786 | −0.09929554 |
| NNMT | −0.1886067 | 1.6402351 | −0.62415093 | −0.5797558 |
| NR4A3 | −0.24002463 | 1.30827564 | −0.50303496 | −0.58789002 |
| NTRK2 | −1.00226189 | −0.12777169 | 0.69034178 | 2.05883128 |
| NTS | −0.67695536 | 0.11676859 | 0.83353897 | 2.50807266 |
| OLFML3 | −0.4126521 | 0.95819848 | −0.30628062 | −0.44250578 |
| PDGFRL | −0.44480374 | 1.19873582 | −0.16399951 | −0.1862348 |
| PDPN | 0.30356845 | 0.63656203 | −1.66343797 | 0.37567565 |
| PGLYRP4 | 0.88505084 | −0.61541037 | −0.33073663 | −0.53271523 |
| PLAC8 | −0.9620995 | 0.02425181 | 2.62687439 | −0.06422655 |
| PNLIPRP3 | 1.57187425 | −0.27236449 | −0.23471355 | −0.27185475 |
| PRAME | −0.69486884 | −0.42350375 | −0.55356105 | 1.405513 |
| RAB38 | 0.73827 | −0.53329397 | −0.44622498 | −0.52110327 |
| RAB6B | −0.31507222 | 0.03054956 | −0.1395046 | 0.86236765 |
| RARRES2 | −0.70373832 | 1.68811559 | −0.00931006 | −0.44940697 |
| RASSF4 | −0.39713767 | 1.21499992 | 0.16999988 | −0.59789479 |
| RGS16 | −0.33545592 | 1.25137566 | 0.01059367 | −0.05440816 |
| RGS20 | 0.98685367 | −0.12811884 | −1.29763847 | −0.37923251 |
| SERPINE1 | 0.2810981 | 0.78397534 | −1.50878669 | −0.09188151 |
| SFRP2 | −0.59354926 | 1.8237165 | −0.09747845 | −0.54071748 |
| SFRP4 | −0.82161397 | 3.416405 | −0.6919744 | −0.47959283 |
| SH3BGRL2 | −0.07755835 | −0.21264704 | 1.19251555 | −0.16710067 |
| SPINK6 | 1.89381372 | −0.8741867 | −1.15269841 | −0.53103374 |
| SPON1 | −0.26198125 | 1.96316318 | −0.31661166 | −0.47379678 |
| ST6GALNAC1 | 0.02963621 | −0.6947042 | 1.97280732 | −0.64699349 |
| TIMP1 | −0.42985033 | 1.12157327 | −0.23141444 | −0.35727128 |
| TMPRSS11B | 0.09453679 | −1.276955 | 4.01044768 | −0.66989218 |
| TMPRSS2 | −0.70192603 | −0.44385887 | 1.80509878 | −0.22099086 |
| UCHL1 | −1.11166132 | 0.01624072 | 0.44356831 | 1.71691127 |
| VCAN | −0.54948326 | 1.40546253 | −0.72685015 | 0.06185013 |

TABLE 12
Top 421 Gene signatures associated with the Basal, Mesenchymal,
Atypical, and Classical head and neck cancer subtypes

| Gene | Basal | Mesenchymal | Atypical | Classical |
|---|---|---|---|---|
| ABCA12 | 1.21E+000 | −0.950148924 | −0.393193535 | −5.61E−002 |
| ABCC1 | 1.38E−001 | −0.344406848 | −0.096857836 | 1.09E+000 |
| ABCC5 | −2.54E−001 | −0.451220573 | 0.277283771 | 8.54E−001 |
| ACSL5 | −1.47E−001 | 0.489959852 | 0.332493996 | −8.58E−001 |
| ACTA2 | −3.20E−001 | 1.050673029 | −0.236968617 | −3.13E−001 |
| ACTA2 | −4.65E−001 | 1.065454518 | −0.197492895 | −4.17E−001 |
| ADAM23 | −1.58E−001 | −0.284388836 | −0.039419379 | 1.64E+000 |
| ADAMTS2 | −1.87E−001 | 0.812777596 | −0.65855743 | −2.49E−001 |
| ADCY10 | −4.99E−002 | 0.016836912 | −0.0698643 | 4.73E−001 |
| AEBP1 | −3.82E−001 | 1.120020446 | −0.565984094 | −1.84E−001 |
| AIF1 | −3.28E−001 | 1.005449659 | 0.063437088 | −9.13E−001 |
| AIM1 | 5.56E−001 | −0.322890127 | 0.184168006 | −7.86E−001 |
| AKR1C1 | 1.10E−001 | −0.96795677 | 0.069668525 | 2.59E+000 |
| AKR1C3 | 5.27E−002 | −0.907348052 | −0.126891654 | 1.68E+000 |
| ALDH1A1 | −1.14E+000 | −0.269442782 | 2.438312649 | 2.82E+000 |
| ALOX5 | −3.62E−001 | 0.723336432 | 0.35602346 | −9.50E−001 |
| AMY1A | −4.29E−001 | 0.343379033 | 1.407517327 | −2.95E−001 |
| AMY2A | −2.94E−001 | 0.183748818 | 1.337762694 | −2.66E−001 |
| ANGPTL2 | −4.52E−001 | 1.086462864 | −0.295391517 | −2.24E−001 |
| ANKRD57 | 7.49E−001 | −0.655400763 | −0.246399809 | −6.17E−002 |
| APOL3 | −1.44E−002 | 0.15308668 | 0.286870296 | −9.81E−001 |
| APOLD1 | −2.97E−001 | 0.971440981 | −0.311515645 | −6.26E−001 |
| AQP3 | 3.90E−001 | −1.114290125 | 0.423704141 | −1.66E+000 |
| ARHGAP4 | −7.84E−001 | 0.584439323 | 1.042630893 | −6.82E−001 |
| ARMCX2 | −9.40E−001 | 0.358433701 | 0.084653447 | 2.99E−001 |
| ARMCX6 | −8.88E−001 | −0.215160715 | 0.339173819 | 3.27E−001 |
| ATP10A | −3.55E−001 | 0.997851564 | 0.041451716 | −4.52E−001 |
| ATP13A4 | −1.73E−001 | −0.648213399 | 1.527935514 | −1.79E−001 |
| ATP2B1 | −3.09E−003 | −0.115664063 | −0.243298764 | 6.58E−001 |
| ATP6V0A4 | 3.70E−002 | −0.408430056 | 1.90064795 | −1.52E−001 |
| ATP6V1D | 4.99E−001 | −0.234720733 | −0.04507946 | −2.31E−001 |
| BBOX1 | 9.67E−001 | −0.278776567 | 0.549622472 | −1.03E+000 |
| BEX2 | −1.01E+000 | −0.417678769 | 0.62501062 | 1.28E+000 |
| BGN | 7.29E−002 | 0.708886143 | −0.394615158 | −2.99E−001 |
| BNC1 | 5.62E−001 | −0.395348529 | −0.930228883 | −5.90E−002 |
| C11orf93 | −2.95E−001 | −0.126353086 | 1.30010065 | 2.10E−001 |
| C1orf113 | 7.11E−001 | −0.434313047 | −0.405814047 | −6.29E−001 |
| C1orf115 | −7.97E−001 | 0.010605671 | 0.621300432 | 2.21E−001 |
| C1orf31 | 9.13E−002 | −0.078617147 | −0.274659534 | 8.22E−001 |
| Cl orf38 | −1.96E−001 | 0.874861445 | −0.292423859 | −4.98E−001 |
| C1orf54 | −2.92E−001 | 0.92639232 | 0 | −3.63E−001 |
| C1R | −1.95E−001 | 0.91292909 | −0.157497446 | −2.41E−001 |
| C2 | −1.83E−001 | 0.819574023 | −0.065378702 | −2.49E−001 |
| C21orf81 | −1.02E+000 | −0.390554183 | 2.2240084 | −3.06E−001 |
| C2orf54 | 1.07E−001 | −1.456009377 | 1.253427557 | −1.41E+000 |
| C4orf19 | −4.02E−002 | −0.10528209 | 1.03831652 | −1.68E−001 |
| C6orf168 | −4.37E−001 | −0.065504472 | 0.133522012 | 1.22E+000 |
| CA2 | 7.22E−001 | −0.627105351 | −1.0503179 | −1.87E−001 |
| CABYR | −1.53E−001 | −0.121569488 | 0.238344489 | 1.15E+000 |
| CALB1 | 2.32E−002 | −0.359161113 | −0.262916386 | 3.37E+000 |
| CALD1 | 1.15E−001 | 0.442278144 | −0.895502258 | −2.02E−001 |
| CAND2 | −6.74E−001 | 0.296517633 | 0.744767695 | 4.95E−001 |
| CASK | 6.51E−003 | −0.216552123 | −0.074779265 | 8.59E−001 |
| CASP4 | 7.88E−001 | 0.196299843 | −0.315306251 | −8.59E−001 |
| CAV1 | 3.76E−001 | 0.303364619 | −1.443550652 | −3.98E−001 |
| CCDC74B | −8.17E−001 | 0.29084425 | 0.183772135 | 3.11E−001 |
| CCL19 | −9.77E−001 | 1.207819435 | 0.967289143 | −1.88E+000 |
| CCL2 | −3.93E−001 | 0.93893577 | −0.120667557 | −5.92E−001 |
| CCL26 | −1.03E−001 | 0.083962352 | −0.157271167 | 1.06E+000 |
| CCR7 | −4.45E−001 | 1.153506447 | 0.550936409 | −1.49E+000 |
| CCRL2 | 5.78E−002 | 0.768607143 | −0.341197435 | −2.25E−001 |
| CD14 | −9.28E−003 | 0.559022187 | −0.289945262 | −8.11E−001 |
| CD2 | −4.90E−001 | 0.71820187 | 0.447225533 | −1.81E+000 |
| CD48 | −2.55E−001 | 0.878807475 | 0.853103404 | −9.81E−001 |
| CD52 | −4.00E−001 | 0.714508082 | 0.604319566 | −1.20E+000 |
| CD74 | −4.28E−001 | 0.863425454 | 0.21401578 | −1.64E+000 |
| CDA | 9.34E−001 | −0.522369638 | −0.503438682 | −4.75E−001 |
| CDKN2B | 7.14E−001 | −0.367262427 | 0.403350772 | −1.27E+000 |
| CEACAM1 | −6.30E−002 | −0.479458098 | 1.770844652 | −2.08E−001 |
| CEACAM5 | 1.85E−001 | −1.195283878 | 1.617954188 | −2.46E−001 |
| CEACAM7 | 1.71E−001 | −0.633745355 | 1.53102362 | −3.41E−001 |
| CFB | 4.50E−002 | 0.467813126 | 0.21928251 | −1.20E+000 |
| CHPT1 | −7.19E−001 | 0.368415397 | 0.628036057 | 2.54E−002 |
| CHRDL2 | −1.66E−001 | 0.926154234 | 0.248935382 | −3.49E−001 |
| CHST7 | −1.30E−001 | −0.04841803 | −0.080050754 | 1.14E+000 |
| CIITA | −8.39E−002 | 0.43117938 | 0.060245327 | −8.19E−001 |
| CLCA4 | −2.36E−002 | −1.045580371 | 2.054256351 | −1.06E+000 |
| CLCN2 | −1.27E−001 | −0.184905444 | 0.068742005 | 7.66E−001 |
| CLDN10 | −5.99E−001 | −0.194873873 | 2.572600311 | 2.63E−001 |
| CLDN7 | −3.58E−001 | −0.853776492 | 0.808855878 | 9.01E−002 |
| CLIC3 | 5.95E−001 | −1.129656348 | 0.953548178 | −1.15E+000 |
| CNN1 | −5.54E−001 | 1.477090921 | −0.204070126 | 1.00E−001 |
| COCH | −5.53E−001 | 0.15654439 | 0.328640387 | 9.48E−001 |
| COL11A1 | 6.69E−003 | 1.945536129 | −1.412645754 | 6.23E−003 |
| COL12A1 | 1.16E−001 | 0.738700842 | −1.466283367 | −1.17E−001 |
| COL17A1 | 6.90E−001 | −0.010801932 | −0.499556185 | −2.82E−001 |
| COL1A2 | −6.65E−002 | 1.142146975 | −0.886236696 | −2.00E−001 |
| COL3A1 | −1.06E−001 | 1.244308903 | −0.740863105 | −5.81E−002 |
| COL5A1 | −7.35E−002 | 1.151596964 | −1.16498573 | −2.77E−001 |
| COL5A2 | −5.48E−002 | 0.980609593 | −1.116893849 | 8.42E−002 |
| COL6A2 | −2.49E−001 | 0.923534822 | −0.544214498 | −3.20E−001 |
| COL6A3 | −1.15E−001 | 0.851002646 | −0.675939468 | −7.27E−002 |
| COL8A1 | −6.06E−002 | 1.121631196 | −0.948411973 | 7.04E−002 |
| COLEC11 | −5.80E−001 | 0.378855189 | 0.342890781 | 4.25E−001 |
| COLEC12 | −3.74E−001 | 1.164203679 | −0.182241943 | −1.78E−001 |
| COMP | −4.50E−001 | 2.292566011 | 0.202252055 | −7.04E−001 |
| CRNN | 5.75E−001 | −2.790276929 | 1.879728017 | −1.74E+000 |
| CRYM | −9.22E−002 | −0.254687144 | 1.347846187 | −2.41E−001 |
| CSNK1A1L | 5.91E−001 | −0.332572255 | −0.258350663 | −2.30E−001 |
| CSNK1A1P | 5.77E−001 | −0.312049609 | −0.235780895 | −2.38E−001 |
| CSTA | 5.04E−001 | −1.487675475 | 0.589934632 | −5.30E−001 |
| CSTB | 3.90E−001 | −1.275928073 | 1.019320401 | −6.19E−001 |
| CT45A1 | −4.54E−001 | 0.282088446 | −0.435512052 | 2.92E+000 |
| CTGF | −3.62E−003 | 0.859044845 | −0.605880857 | −3.19E−001 |
| CTSK | −2.20E−001 | 0.902699745 | −0.280195695 | −1.45E−001 |
| CTSL1 | 4.65E−001 | 0.224164355 | −1.098862848 | −2.30E−001 |
| CWH43 | 1.54E+000 | −1.742619114 | 0.064535374 | −1.39E+000 |
| CXCL12 | −9.45E−001 | 2.140568245 | 0.25053663 | −1.25E+000 |
| CXCL17 | −1.79E−001 | −0.362373317 | 0.871895624 | −4.67E−001 |
| CXCR4 | −7.42E−001 | 0.900251746 | 0.284920242 | −8.27E−001 |
| CYBB | −2.38E−001 | 0.949327445 | 0.01247299 | −6.58E−001 |
| CYP26A1 | −5.73E−001 | −0.073092383 | 0.42973868 | 2.21E+000 |
| CYP2E1 | −2.29E−001 | −0.273556366 | 1.824106885 | −2.46E−001 |
| CYP3A5 | −3.06E−003 | −0.987738382 | 2.046142019 | −6.78E−001 |
| CYP4B1 | −5.31E−001 | −0.41998758 | 2.664269435 | −1.02E+000 |
| CYP4F11 | 1.42E−001 | −0.524232331 | 0.302582927 | 2.21E+000 |
| D4S234E | 1.18E+000 | −1.243071464 | −0.301692366 | −3.31E−001 |
| DAAM1 | 5.90E−001 | −0.336180228 | −0.303712221 | −2.75E−001 |
| DAB2 | −1.39E−001 | 0.56017232 | −0.126305333 | −1.20E−001 |
| DACT1 | −3.61E−001 | 1.396057661 | −0.60988427 | −6.10E−002 |
| DCN | −1.05E−001 | 1.344360064 | −0.358097035 | −3.01E−001 |
| DEFB103B | 7.16E−001 | −0.837189761 | 0.024616415 | −6.29E−001 |
| DLX6 | −1.57E−002 | −0.167945944 | −0.081944602 | 4.49E−001 |
| DMKN | 7.10E−001 | −0.456045359 | −0.274110081 | −5.21E−001 |
| DPYSL3 | −9.25E−001 | 0.967653022 | 0.034562746 | 5.49E−001 |
| DSC1 | 1.82E+000 | −0.566220415 | −0.247658391 | 1.01E−002 |
| DSC2 | 1.05E+000 | −0.802860155 | −0.067575541 | 2.19E−002 |
| DSG1 | 1.82E+000 | −0.806739891 | −0.748721684 | −7.39E−001 |
| DUSP14 | 7.54E−001 | −0.164593838 | −0.682547404 | −1.25E−001 |
| ECHDC2 | −4.73E−001 | −0.09680909 | 0.845506751 | −3.53E−001 |
| EFHA2 | −5.39E−001 | 0.983489516 | 0.714371694 | −2.40E−002 |
| EMP1 | −4.65E−002 | −0.508642742 | 0.929375326 | −3.32E−001 |
| ENAH | 3.66E−001 | 0.126332879 | −0.725650768 | −3.18E−002 |
| EPCAM | −3.69E−001 | −0.30894192 | 0.369204313 | 1.31E+000 |
| EPGN | 1.82E+000 | −0.6381393 | −0.042951356 | −4.68E−001 |
| EPHX2 | −2.95E−001 | −0.42759907 | 1.037638242 | −1.57E−002 |
| EREG | 1.49E+000 | −0.419344153 | −0.828724891 | −1.10E+000 |
| EYA2 | −1.10E+000 | −0.063192112 | 2.462963338 | 2.61E−001 |
| F13A1 | −2.06E−001 | 0.961770797 | −0.123770247 | −7.78E−001 |
| FABP5 | 8.23E−001 | −0.479845895 | −0.568350316 | −3.45E−001 |
| FAM101A | −5.76E−001 | 1.657262199 | −0.338461109 | −2.41E−001 |
| FAM119A | −1.59E−001 | −0.012745809 | −0.066611106 | 6.27E−001 |
| FAM176B | −1.61E−001 | 0.843824636 | −0.156323284 | −2.52E−001 |
| FAM198B | −1.14E−001 | 1.39244454 | −0.38970484 | −3.09E−001 |
| FAM3B | −8.14E−001 | −0.639957464 | 3.950426423 | −3.01E−001 |
| FAM3D | −5.10E−001 | −0.992650089 | 0.95347955 | −3.49E−001 |
| FAM46B | 9.95E−001 | −0.517478836 | 0.239701649 | −1.47E+000 |
| FAM48B2 | 3.34E−002 | −0.061438843 | 1.127843706 | −1.88E−001 |
| FAM71F1 | −1.40E−001 | 0.176350132 | −0.085010284 | 1.18E+000 |
| FAM83A | 9.59E−001 | −0.36992713 | −0.629867112 | −8.49E−002 |
| FAM83B | 7.54E−001 | −0.474890902 | −0.247830538 | 1.07E−001 |
| FBLIM1 | 8.35E−001 | 0.042751069 | −0.623410986 | −2.99E−001 |
| FCER1A | −3.79E−004 | 0.038815521 | 0.689284346 | −1.06E+000 |
| FCGR1A | −1.85E−001 | 0.84696684 | −0.177685474 | −5.03E−001 |
| FCGR1C | −9.75E−002 | 0.99171583 | −0.148322565 | −7.76E−001 |
| FGL2 | −1.38E−001 | 0.585849484 | −0.050457001 | −1.19E+000 |
| FLRT3 | 1.44E+000 | −0.001292334 | −1.653502027 | −7.51E−001 |
| FMO2 | −1.46E−001 | −0.001407586 | 1.454534272 | −4.36E−001 |
| FN1 | −2.84E−001 | 1.200495988 | −0.680625828 | −4.45E−002 |
| FNDC1 | −2.98E−001 | 1.621511951 | −0.790403218 | −6.91E−001 |
| FOXA1 | −8.35E−001 | −0.728894919 | 1.928347421 | 2.93E−001 |
| FOXP1 | −4.29E−002 | 0.296978331 | 0.124054717 | −5.56E−001 |
| FSTL1 | −2.97E−001 | 1.04990607 | −0.5895564 | −7.29E−002 |
| FSTL3 | 4.66E−001 | 0.159592837 | −0.966925955 | 0.00E+000 |
| FUT3 | 8.97E−002 | −0.789402684 | 1.016480721 | −7.39E−001 |
| FUT5 | 1.35E−001 | −0.805645409 | 1.424360829 | −7.62E−001 |
| FUT6 | 8.61E−002 | −0.421906208 | 1.316249504 | −3.14E−001 |
| FUT7 | −7.90E−002 | −0.157973732 | 1.062793383 | −2.71E−001 |
| FYB | 2.05E−002 | 0.826238248 | 0.062676415 | −9.32E−001 |
| FZD7 | −6.11E−001 | 0.1075121 | 0.281277546 | 1.00E+000 |
| GABRP | −3.12E−001 | −0.15934957 | 1.716288795 | −2.71E−001 |
| GALNT12 | −6.05E−001 | −0.284094438 | 1.393745467 | 3.29E−001 |
| GALNT6 | 6.42E−001 | −0.06315521 | −0.593857338 | −2.18E−001 |
| GAS1 | 1.16E−001 | 1.371254269 | −0.725452696 | −1.63E−001 |
| GBP6 | 2.77E−001 | −1.247128333 | 1.083369022 | −1.50E−001 |
| GCNT2 | −5.33E−001 | 0.012055685 | 0.608172602 | 5.23E−001 |
| GCNT3 | −6.87E−002 | −0.322144853 | 1.270432155 | 2.03E−001 |
| GGT5 | 5.86E−003 | 0.898025205 | −0.491348042 | −6.62E−001 |
| GGTA1 | −7.00E−002 | 0.512066407 | 0.461744488 | −1.39E+000 |
| GIMAP5 | −1.63E−001 | 0.482118654 | 0.343314671 | −1.12E+000 |
| GIMAP8 | −2.19E−002 | 0.665904948 | 0.158309732 | −1.03E+000 |
| GMFG | −4.31E−001 | 1.059175666 | 0.300434155 | −8.79E−001 |
| GNG11 | −8.85E−002 | 1.028699462 | −0.143932691 | −5.37E−001 |
| GPD1L | −1.87E−001 | −0.119665323 | 0.902202893 | −5.51E−017 |
| GPR110 | 5.38E−001 | −2.275284677 | 1.761816909 | −1.49E+000 |
| GPR115 | 6.04E−001 | −0.497623987 | 0.006236637 | −6.46E−001 |
| GPX2 | −5.87E−001 | −0.825890323 | 0.804460854 | 1.98E+000 |
| GRASP | −4.30E−001 | 1.091608781 | 0.016389431 | −3.85E−001 |
| GRHL3 | 4.30E−001 | −1.287770156 | 0.508830019 | −2.17E−001 |
| GSDMC | 1.01E+000 | −0.38696587 | −0.276940216 | −6.43E−001 |
| GSPT2 | −9.47E−001 | 0.031716948 | 0.166420024 | 4.33E−001 |
| GSTA1 | −3.17E−001 | −0.098617521 | 0.941564612 | 1.45E+000 |
| GSTA5 | −4.84E−001 | −0.074616686 | 1.131279584 | 2.14E+000 |
| GSTM2 | −7.22E−001 | −0.022082186 | 0.561979952 | 1.20E+000 |
| GSTM3 | −6.57E−001 | −0.264352657 | 0.336978378 | 1.91E+000 |
| GZMA | 4.55E−002 | 0.452230643 | −0.005311119 | −1.64E+000 |
| GZMK | −4.34E−001 | 0.642294874 | 0.204380268 | −1.37E+000 |
| HAVCR2 | −5.70E−002 | 0.655635781 | −0.108186339 | −2.13E−001 |
| HEY1 | −6.59E−001 | 0.4344292 | 0.010896057 | 1.48E+000 |
| HLA-DMA | −2.19E−001 | 0.695113708 | 0.4216724 | −1.17E+000 |
| HLA-DPA1 | −2.22E−001 | 0.659680433 | 0.351926047 | −1.05E+000 |
| HLA-DPB1 | −3.74E−001 | 0.595640509 | 0.106863499 | −1.14E+000 |
| HLA-DQB1 | −2.46E−001 | 0.456097998 | 0.105134601 | −9.62E−001 |
| HLA-DQB2 | −2.16E−001 | 0.587336292 | 0.097074221 | −1.19E+000 |
| HLA-DRA | −1.59E−001 | 0.49384548 | −0.0019877 | −1.70E+000 |
| HLA-DRB5 | −8.79E−002 | 0.408821096 | 0.065328037 | −8.01E−001 |
| HLF | −4.16E−001 | −0.2107738 | 1.080707583 | 4.60E−002 |
| HOXC9 | −2.96E−001 | 0.160622497 | −0.840930437 | 9.40E−001 |
| HPGD | −9.51E−002 | −0.221819554 | 1.313337434 | −1.69E−001 |
| HS3ST4 | −2.17E−001 | −0.066871907 | 1.009513545 | 5.06E−002 |
| HSD11B1 | 5.89E−003 | 1.084941302 | −0.395927019 | −3.16E−001 |
| HSPB2 | −4.53E−001 | 1.198778446 | 0.090387023 | −5.95E−001 |
| HSPC159 | 1.19E+000 | −0.817370744 | −0.370770166 | −6.33E−001 |
| HTRA3 | −2.16E−001 | 0.712168782 | −0.482745998 | −3.53E−001 |
| ICAM2 | −2.19E−001 | 0.942499285 | 0.495706842 | −8.79E−001 |
| IFFO1 | −2.03E−001 | 1.107885243 | 0.023168847 | −4.41E−001 |
| IGFBP7 | −2.11E−001 | 0.751716204 | −0.492782742 | −2.46E−001 |
| IL18 | 9.13E−001 | −0.534686107 | −0.109637416 | −8.84E−001 |
| IL1F5 | 8.74E−001 | −1.458971317 | −0.325621594 | −6.36E−001 |
| IL21R | −3.98E−001 | 0.707131951 | 0.096541091 | −7.96E−001 |
| IL4I1 | −4.00E−001 | 0.703123196 | 0.013698058 | −5.73E−001 |
| IL6 | −2.01E−001 | 1.836447371 | −1.27590628 | 6.36E−001 |
| INHBA | 4.33E−001 | 0.739095246 | −2.828546637 | −2.59E−001 |
| IRF8 | −1.77E−001 | 0.820674728 | 0.197117289 | −7.93E−001 |
| JAM2 | −3.55E−001 | 0.901604923 | 0.242368553 | −3.87E−001 |
| KCNMB3 | −2.40E−001 | −0.008715607 | 0.004102137 | 4.68E−001 |
| KCTD12 | 4.28E−003 | 0.59076735 | −0.211490302 | −8.97E−001 |
| KIAA1609 | 5.58E−001 | −0.067105624 | −0.514647103 | −4.98E−001 |
| KLK5 | 7.12E−001 | 0.07715915 | −1.305917404 | −1.46E+000 |
| KLK7 | 1.11E+000 | −0.965216685 | 0.048612422 | −1.45E+000 |
| KRT10 | 1.23E+000 | −0.448967067 | −0.189985135 | −3.29E−001 |
| KRT13 | 6.01E−002 | −0.124661297 | 0.898367593 | −5.32E−001 |
| KRT15 | −3.26E−001 | −0.098274628 | 0.965763844 | −4.68E−001 |
| KRT19 | −2.06E+000 | −0.651580405 | 1.923935022 | 9.59E−001 |
| KRT24 | 8.37E−001 | −2.241276957 | 2.206810114 | −9.18E−001 |
| KRT4 | 3.39E−001 | −1.463831609 | 1.613425676 | −6.65E−001 |
| KRT75 | 1.28E+000 | −0.507514716 | −0.073749228 | −9.49E−001 |
| KRT79 | 9.15E−001 | −0.949427397 | −0.173583825 | −4.34E−001 |
| LAMA4 | −1.80E−001 | 0.729756482 | −0.461642439 | −9.31E−002 |
| LGALS1 | 5.78E−002 | 0.506926904 | −0.933634247 | 1.01E−001 |
| LHFP | −1.97E−001 | 0.795241179 | −0.371213406 | −4.96E−001 |
| LMO4 | −2.39E−001 | 0.004579139 | 0.916506975 | −3.16E−001 |
| LOC284233 | −8.66E−002 | −0.075513205 | 0.601901656 | −2.79E−002 |
| LOC643008 | 5.94E−002 | −0.513693947 | 1.195838212 | −5.40E−001 |
| LPAR3 | 7.10E−001 | −0.201561335 | −0.602439428 | −4.30E−002 |
| LPPR1 | −2.49E−001 | 0.029149685 | −0.010962796 | 5.39E−001 |
| LRIG1 | −8.48E−001 | 0.465659751 | 0.309896986 | 3.03E−001 |
| LRP12 | −1.38E−002 | 0.003655851 | −0.655861118 | 8.33E−001 |
| LST1 | −1.45E−001 | 0.756034756 | −0.117600035 | −6.91E−001 |
| LTB | −2.81E−001 | 0.857247403 | 0.969622399 | −1.22E+000 |
| LTF | −8.54E−001 | 0.019220535 | 3.301548333 | −6.53E−001 |
| LXN | −9.23E−001 | 0.568056227 | 0.344398221 | −1.10E−001 |
| LYPD5 | 1.06E+000 | −0.208231225 | −0.339530658 | −3.28E−001 |
| MAGED4B | −7.95E−001 | 0.484853086 | −0.015310825 | 7.47E−001 |
| MAL | −1.33E−001 | −0.925367297 | 4.447704447 | −4.27E−001 |
| MANSC1 | −3.78E−001 | −0.407750833 | 1.127746846 | 3.01E−001 |
| MARVELD1 | −1.52E−001 | 0.435500631 | −0.83800127 | 3.05E−002 |
| MDK | −6.50E−001 | 0.483205903 | 0.479151689 | 4.71E−001 |
| MEF2C | −3.93E−001 | 1.180427339 | 0.298298448 | −6.15E−001 |
| MEI1 | −4.78E−001 | 1.234899004 | 0.863330186 | −1.13E+000 |
| MGP | −6.04E−001 | 1.329360455 | 0.213618556 | −5.99E−001 |
| MGST2 | −1.06E−002 | −0.244645467 | 0.800977315 | −1.76E−001 |
| MICALCL | 1.81E+000 | −0.118416415 | −0.943748767 | −4.85E−001 |
| MMP1 | 6.72E−001 | 0.979687473 | −1.878844495 | −2.20E−001 |
| MMP28 | 5.22E−001 | 0.356597648 | −0.146391962 | −1.36E+000 |
| MMP3 | 1.08E+000 | 1.715223971 | −1.581331989 | −4.47E−001 |
| MOBKL2B | 1.01E+000 | −0.016470486 | −0.313515306 | −4.65E−001 |
| MPPED1 | −1.53E−001 | −0.113987147 | 0.373664548 | 1.09E+000 |
| MRAP2 | −4.46E−001 | −0.132169834 | 0.343554635 | 1.24E+000 |
| MRAS | −2.02E−001 | 0.717206907 | −0.196574497 | −2.38E−001 |
| MS4A1 | −3.20E−001 | 0.407476329 | 1.143888025 | −3.35E−001 |
| MS4A4A | −6.56E−002 | 1.109400548 | −0.258099264 | −4.01E−001 |
| MT1B | 4.47E−001 | 0.970442168 | −1.359072766 | −6.71E−001 |
| MT1L | 5.17E−001 | 1.145063942 | −1.314245508 | −6.17E−001 |
| MT2A | 4.88E−001 | 0.820682518 | −1.450635935 | −5.60E−001 |
| MUC20 | −6.57E−001 | −0.870145692 | 1.23306638 | 3.22E−001 |
| MUC4 | −4.09E−001 | −0.625152662 | 1.256719702 | −1.20E−001 |
| MXRA5 | −3.71E−001 | 0.719318376 | −0.400035237 | −3.65E−001 |
| MXRA8 | −6.16E−001 | 1.045510927 | −0.290165861 | −3.17E−001 |
| MYL9 | −1.89E−001 | 0.690048362 | −0.227352552 | −8.09E−002 |
| MYO5C | −4.22E−001 | −0.365249698 | 1.424082092 | −1.56E−001 |
| NAPSB | −2.56E−001 | 0.582354582 | 0.539135244 | −1.12E+000 |
| NDFIP2 | 5.93E−001 | −0.456026039 | −0.188857234 | −1.91E−001 |
| NEXN | 1.14E−001 | 1.010894729 | −0.603585775 | −4.07E−001 |
| NID2 | −1.94E−001 | 0.912366445 | −0.812837859 | −9.93E−002 |
| NLRP3 | 2.94E−001 | 0.846170471 | −0.587313005 | −8.09E−001 |
| NMU | 8.35E−002 | −1.050425878 | 1.327115427 | −6.36E−001 |
| NNMT | −1.89E−001 | 1.640235097 | −0.624150926 | −5.80E−001 |
| NR4A3 | −2.40E−001 | 1.308275635 | −0.503034963 | −5.88E−001 |
| NT5E | 3.93E−001 | 0.634629418 | −1.017419268 | −5.73E−002 |
| NTNG2 | −1.80E−001 | 0.913473705 | −0.237999633 | −2.29E−001 |
| NTRK2 | −1.00E+000 | −0.127771687 | 0.690341777 | 2.06E+000 |
| NTS | −6.77E−001 | 0.116768592 | 0.833538965 | 2.51E+000 |
| OLFML2B | −1.41E−001 | 0.875062615 | −0.490966953 | −1.94E−001 |
| OLFML3 | −4.13E−001 | 0.958198475 | −0.306280624 | −4.43E−001 |
| ORC6L | 1.65E−002 | −0.157330082 | −0.25137985 | 9.88E−001 |
| OTUD1 | 6.36E−001 | −0.138747676 | −0.270354361 | −3.17E−001 |
| P4HA2 | 2.97E−001 | 0.372331755 | −0.840507231 | 3.25E−002 |
| PANX1 | 5.20E−001 | 0.048483572 | −0.746129946 | −2.56E−001 |
| PAQR5 | 7.70E−001 | −0.371418661 | −0.543595823 | −1.16E−001 |
| PCDH7 | 9.52E−001 | 0.092326466 | −0.507123335 | −6.44E−001 |
| PCOLCE | −4.99E−001 | 0.743430165 | −0.193312325 | −1.68E−001 |
| PDE6B | −7.21E−001 | 0.36501016 | 0.671461399 | 6.12E−002 |
| PDGFRL | −4.45E−001 | 1.198735819 | −0.163999513 | −1.86E−001 |
| PDPN | 3.04E−001 | 0.636562033 | −1.66343797 | 3.76E−001 |
| PDZD2 | 3.59E−001 | −0.212871078 | 0.25947319 | −5.98E−001 |
| PFN2 | −8.79E−002 | −0.302079218 | −0.199807355 | 9.70E−001 |
| PGLYRP4 | 8.85E−001 | −0.615410366 | −0.330736627 | −5.33E−001 |
| PIR | −2.49E−001 | −0.641982926 | 0.546876899 | 1.24E+000 |
| PITX1 | 1.27E−001 | −0.516701658 | 0.850515437 | −4.17E−001 |
| PKP1 | 4.91E−001 | −0.997312703 | −0.386056098 | −1.07E−001 |
| PLAC8 | −9.62E−001 | 0.024251813 | 2.626874385 | −6.42E−002 |
| PLAU | 1.53E−001 | 0.620269115 | −0.922246794 | −7.32E−002 |
| PLCE1 | −7.80E−001 | 0.381542762 | 0.62464978 | 8.11E−002 |
| PMP22 | −2.93E−001 | 0.750200735 | −0.344285191 | −2.47E−001 |
| PNLIPRP3 | 1.57E+000 | −0.272364493 | −0.234713546 | −2.72E−001 |
| POSTN | −5.36E−002 | 1.724039041 | −1.131864401 | −8.42E−002 |
| PP14571 | −2.53E−001 | −0.272608281 | 1.632484639 | −1.87E−002 |
| PPAPDC3 | −2.03E−001 | 1.359125039 | −0.230492835 | 6.48E−002 |
| PPIF | 9.33E−001 | −0.206867623 | −0.565759743 | −1.96E−001 |
| PPL | 1.56E−001 | −0.903933684 | 0.923949495 | −3.69E−001 |
| PPP2R2C | 7.86E−001 | −0.810020889 | −0.488639207 | −1.49E−001 |
| PRAME | −6.95E−001 | −0.423503751 | −0.553561053 | 1.41E+000 |
| PRR15L | −4.94E−002 | −0.488258756 | 1.55338868 | −1.61E−001 |
| PRSS27 | 5.48E−001 | −0.866789057 | 1.401474857 | −6.01E−001 |
| PSCA | 8.90E−002 | −0.203947184 | 1.026601633 | −6.95E−002 |
| PTN | −1.15E+000 | −0.44442481 | 1.30377896 | 6.46E−001 |
| PTX3 | −2.24E−001 | 1.04068096 | −0.603490611 | −3.23E−001 |
| RAB38 | 7.38E−001 | −0.533293969 | −0.446224982 | −5.21E−001 |
| RAB6B | −3.15E−001 | 0.030549563 | −0.139504601 | 8.62E−001 |
| RAET1E | 8.37E−001 | −0.889120545 | 0.421788242 | −1.07E+000 |
| RARRES2 | −7.04E−001 | 1.688115591− | 0.009310057 | −4.49E−001 |
| RASAL3 | −4.59E−001 | 0.883390902 | 0.620088517 | −9.12E−001 |
| RASSF4 | −3.97E−001 | 1.214999917 | 0.169999883 | −5.98E−001 |
| RECK | −3.48E−001 | 0.915397213 | −0.137288515 | −2.27E−001 |
| RFTN1 | 1.66E−001 | 0.424058022 | −0.698685897 | −9.20E−001 |
| RGMA | −4.86E−001 | −0.088937863 | 0.812784065 | 6.84E−001 |
| RGS16 | −3.35E−001 | 1.251375664 | 0.010593665 | −5.44E−002 |
| RGS20 | 9.87E−001 | −0.128118835 | −1.29763847 | −3.79E−001 |
| RIMKLA | −3.58E−001 | −0.096768783 | 0.065960074 | 8.03E−001 |
| RNASE1 | −9.30E−002 | 0.702909505 | −0.175733976 | −3.63E−001 |
| RRAS2 | 7.80E−001 | −0.036759041 | −0.791859069 | −8.88E−002 |
| S100A7A | 1.08E+000 | −0.970462557 | −0.187283443 | −9.67E−001 |
| S100B | −2.78E−001 | 0.517766965 | 0.245907777 | −9.52E−001 |
| SAMD9 | 7.38E−001 | −0.41885608 | −0.030798084 | −4.10E−001 |
| SCEL | 5.34E−001 | −1.941481153 | 1.440181122 | −9.78E−001 |
| SCN1A | −1.41E−001 | 0.015081415 | −0.012787683 | 6.82E−001 |
| SCNN1A | −9.99E−002 | −0.846940154 | 1.013202708 | 2.98E−001 |
| SERPINB5 | 7.15E−001 | −0.658371864 | −0.528747928 | −2.00E−001 |
| SERPINB7 | 1.01E+000 | −0.621534154 | −0.434656433 | −1.79E−001 |
| SERPINB8 | 8.59E−001 | −0.295912049 | −0.090311073 | −3.49E−001 |
| SERPINE1 | 2.81E−001 | 0.783975335 | −1.508786685 | −9.19E−002 |
| SFRP1 | 4.55E−001 | 0.486706716 | −0.067784494 | −1.55E+000 |
| SFRP2 | −5.94E−001 | 1.823716495 | −0.097478448 | −5.41E−001 |
| SFRP4 | −8.22E−001 | 3.416405004 | −0.691974401 | −4.80E−001 |
| SGEF | −5.40E−001 | −0.249424711 | 0.724305259 | 7.67E−001 |
| SH2D5 | 6.95E−001 | 0.159615404 | −1.188955617 | −3.18E−001 |
| SH3BGRL2 | −7.76E−002 | −0.212647039 | 1.192515554 | −1.67E−001 |
| SLAMF7 | 3.84E−001 | 0.587330427 | 0.053700803 | −9.68E−001 |
| SLC2A9 | 7.53E−001 | −0.128242655 | −0.350632989 | −2.48E−001 |
| SLC31A2 | 6.29E−001 | 0.162950411 | −0.737063883 | −9.58E−001 |
| SLC37A1 | −1.44E−001 | −0.023661789 | 0.74120191 | −1.70E−001 |
| SLC6A10P | −3.74E−002 | −0.483363477 | −0.007945994 | 1.04E+000 |
| SMARCD3 | −5.76E−001 | 0.628277357 | 0.240236806 | 2.57E−001 |
| snai1 | −7.37E−001 | 0.241186675 | −1.146558906 | −8.84E−001 |
| SNAI2 | 3.20E−001 | 0.286204446 | −0.824516111 | 2.60E−001 |
| SOD3 | −4.30E−001 | 0.993904834 | 0.268514443 | −5.54E−001 |
| SORBS2 | −1.59E−001 | 0.348002018 | 1.340047916 | −4.55E−001 |
| SOSTDC1 | −4.09E−001 | 0.034772276 | 0.35515764 | 1.72E+000 |
| SOX2 | −3.77E−002 | 0.364903257 | 2.043139358 | 2.80E+000 |
| SPARC | −2.60E−001 | 1.108141191 | −0.612318629 | −8.48E−002 |
| SPINK5 | 3.63E−001 | −1.45981084 | 1.257121929 | −1.57E+000 |
| SPINK6 | 1.89E+000 | −0.874186699 | −1.152698411 | −5.31E−001 |
| SPON1 | −2.62E−001 | 1.963163183 | −0.316611655 | −4.74E−001 |
| SPRR2G | 1.35E+000 | −0.207539078 | −1.438005447 | −7.50E−001 |
| ST6GALNAC1 | 2.96E−002 | −0.694704204 | 1.972807315 | −6.47E−001 |
| STAB1 | −8.66E−002 | 0.412693496 | 0 | −9.30E−001 |
| SYTL3 | −3.12E−002 | 0.353209685 | 0.221679767 | −8.27E−001 |
| TAGLN | −2.19E−001 | 0.982287635 | −0.190960072 | −2.16E−001 |
| TBC1D10C | −3.37E−001 | 0.807839442 | 0.907144543 | −1.32E+000 |
| TCEA3 | −4.09E−001 | −0.318754503 | 0.824220959 | −1.16E−002 |
| TFRC | −2.28E−001 | −0.169600761 | 0.068624092 | 9.10E−001 |
| TGFB3 | −2.16E−001 | 0.570768508 | −0.283777878 | −3.48E−001 |
| TGFBI | 5.38E−001 | 0.434142377 | −1.261382146 | 7.56E−002 |
| TGM3 | 1.10E+000 | −1.531682998 | 1.689610881 | −3.61E−001 |
| THBS2 | −2.41E−002 | 1.166108448 | −1.464472491 | −2.90E−001 |
| THSD1 | 6.61E−001 | 0.125693416 | −0.815532483 | −1.68E−001 |
| THY1 | −2.15E−001 | 0.900786117 | −0.351780204 | −2.03E−001 |
| TIMP1 | −4.30E−001 | 1.121573271 | −0.231414439 | −3.57E−001 |
| TLR5 | −2.25E−001 | −0.09643289 | 0.937118222 | −1.75E−001 |
| TMEM154 | 5.86E−001 | −0.991609548 | −0.017129383 | −5.12E−001 |
| TMEM176B | −2.70E−001 | 0.857396566 | 0.013984741 | −8.46E−001 |
| TMEM51 | 1.38E−001 | 0.24390605 | −0.122716246 | −6.21E−001 |
| TMPRSS11A | −8.28E−002 | −0.282031761 | 1.138391199 | −6.80E−002 |
| TMPRSS11B | 9.45E−002 | −1.276954995 | 4.010447682 | −6.70E−001 |
| TMPRSS2 | −7.02E−001 | −0.44385887 | 1.805098776 | −2.21E−001 |
| TNFRSF12A | 2.73E−001 | 0.289677633 | −1.293145772 | −8.01E−002 |
| TPM1 | 2.40E−002 | 0.718612311 | −0.940222411 | −8.12E−002 |
| TPM2 | −1.70E−001 | 0.926795312 | −0.652770846 | −4.85E−002 |
| TRAF3IP3 | −5.84E−001 | 0.776529718 | 0.884191079 | −1.29E+000 |
| TRPV2 | −2.67E−001 | 0.796700139 | 0.056708965 | −4.71E−001 |
| TUBB2A | 7.70E−001 | −0.416790337 | −0.238473276 | −3.38E−001 |
| TXNRD1 | −1.10E−001 | −0.117942165 | −0.099655341 | 1.24E+000 |
| UCHL1 | −1.11E+000 | 0.01624072 | 0.443568311 | 1.72E+000 |
| UPP1 | 9.29E−001 | −0.462177801 | −0.995891621 | −1.77E−001 |
| VASN | 5.96E−002 | 0.662201692 | 0.198931984 | −5.86E−001 |
| VAV3 | 4.66E−005 | −0.156264926 | 0.63234442 | −8.89E−001 |
| VCAN | −5.49E−001 | 1.405462528 | −0.726850148 | 6.19E−002 |
| VEGFC | 1.53E+000 | 1.166583704 | −0.928987979 | −4.13E−001 |
| VGLL3 | −1.49E−001 | 1.08110238 | −0.415371405 | −2.77E−002 |
| VIM | −4.16E−001 | 0.916888837 | −0.220035876 | −2.27E−001 |
| WDFY4 | −1.59E−001 | 0.358696707 | 0.136868747 | −6.87E−001 |
| WISP2 | −2.50E−001 | 1.194329758 | 0.02677436 | −2.25E−001 |
| WNT4 | 7.68E−001 | −0.295506514 | 0.253170282 | −6.08E−001 |
| ZBED3 | −5.64E−001 | 0.554377602 | 0.334041109 | 2.86E−002 |
| ZDHHC2 | −6.16E−001 | −0.019832412 | 0.348255585 | 6.49E−001 |
| ZEB2 | −1.36E−001 | 0.659646956 | −0.097190947 | −2.19E−001 |
| ZIC1 | −6.15E−001 | 0.226137728 | 0.26693427 | 1.65E+000 |
| ZNF521 | −4.19E−001 | 0.826159968 | 0.035153403 | −2.56E−001 |
| ZNF639 | −1.65E−001 | −0.026730814 | −0.160356293 | 6.85E−001 |

### 6.4. References (Sec. 2.2 and 6.3)

1. R. Siegel, E. Ward, 0. Brawley, A. Jemal, Cancer Statistics 2011. CA: Cancer J. Clin. 61, 212-236 (2011).
2. H. Mehanna, C. M. L. West, C. Nutting, V. Paleri, Head and Neck Cancer—Part 2: Treatment and Prognostic Factors. Brit. Med. J. 341, c4690 (2010).
3. Ang, K. K., Harris, J., Wheeler, R., Weber, R., Rosenthal, D. I., Nguyen-Tan, P. F., Westra, W. H., Chung, C. H., Jordan, R. C., Lu, C., Kim, H., Axelrod, R., Silverman, C. C., Redmond, K. P., Gillison, M. L., Human Papillomavirus and Survival of Patients with Oropharyngeal Cancer. New Eng. J. Med. 363, 24-35 (2010).
4. AJCC Cancer Staging Manual, S. B. Edge, D. R. Byrd, C. C. Compton, A. G. Fritz, F. L. Greene, A. Trotti, Eds. (Springer, New York, 2009).
5. C. M. Perou, T. Sorlie, M. B. Eisen, M. van de Rijn, S. S. Jeffrey, C. A. Rees, J. R. Pollack, D. T. Ross, H. Johnsen, L. A. Akslen, O. Fluge, A. Pergamenschikov, C. Williams, S. X. Zhu, P. E. Lonning, A-L. Borresen-Dale, P. O. Brown, D. Botstein, Molecular Portraits of Human Breast Tumors. Nature. 406, 747-752 (2000).
6. T. Sorlie, R. Tibshirani, J. Parker, T. Hastie, J. S. Marron, A. Nobel, S. Deng, H. Johnsen, R. Pesich, S. Geisler, J. Demeter, C. M. Perou, P. E. Lenning, P. O. Brown, A-L. Borresen-Dale, D. Botstein, Repeated Observations of Breast Tumor Subtypes in Independent Gene Expression Data Sets. Proc. Nat. Acad. Sci. 100(14), 8418-8423 (2003).
7. M. D. Wilkerson, X. Yin, K. A. Hoadley, Y. Liu, M. C. Hayward, C. R. Cabanski, K. Muldrew, C. R. Miller, S. H. Randell, M. A. Socinski, A. M. Parsons, W. K. Funkhouser, C. B. Lee, P. J. Roberts, L. Thorne, P. S. Bernard, C. M. Perou, D. N. Hayes, Lung Squamous Cell Carcinoma mRNA Expression Subtypes are Reproducible, Clinically Important, and Correspond to Normal Cell Types. Clin. Cancer Res. 16(19), 4864-4875 (2010).
8. C. H. Chung, J. S. Parker, G. Karaca, J. Wu, W. K. Funkhouser, D. Moore, D. Butterfoss, D. Xiang, A. Zanation, X. Yin, W. W. Shockley, M. C. Weissler, L. G. Dressler, C. G. Shores, W. G. Yarbrough, C. M. Perou, Molecular Classification of Head and Neck Squamous Cell Carcinoma Using Patterns of Gene Expression. Cancer Cell. 5(5), 489-500 (2004).
9. M. D. Wilkerson, D. N. Hayes, ConsensusClusterPlus: A Class Discovery Tool with Confidence Assessments and Item Tracking. Bioinformatics. 26(12), 1572-1573 (2010).
10. Y. Liu, D. N. Hayes, A. Nobel, J. S. Marron, Statistical Significance of Clustering for High-Dimension, Low-Sample Size Data. J. Amer. Stat. Assoc. 103(483), 1281-1293 (2008).
11. Gillison, M. L., Koch, W. M., Capone, R. B., Spafford, M., Westra, W. H., Wu, L., Zahurak, M. L., Daniel, R. W., Viglione, M., Symer, D. E., Shah, K. V., Sidransky, D., Evidence for a Causal Association Between Human Papillomavirus and a Subset of Head and Neck Cancers. J. Nat. Cancer Inst. 92 (9) 709-720 (2000).
12. Patmore, H. S., Cawkwell, L., Stafford, N. D., Greenman, J., Unraveling the Chromosomal Aberrations of Head and Neck Squamous Cell Carcinoma: A Review. Ann. Surg. Oncology. 12 (10) 831-842 (2005).
13. B. Singh, S. K. Gogineni, P. G. Sacks, A. R. Shaha, J. P. Shag, A. Stoffel, P. H. Rao, Molecular Cytogenetic Characterization of Head and Neck Squamous Cell Carcinoma and Refinement of 3q Amplification. Cancer Res. 61, 4506-4513 (2001).
14. The Cancer Genome Atlas Research Network, Comprehensive Genomic Characterization of Squamous Cell Lung Cancers, submitted.
15. D. W. Huang, B. T. Sherman, R. A. Lempicki, Systematic and Integrative Analysis of Large Gene Lists Using DAVID Bioinformatics Resources. Nature Protocols. 4(1), 44-57 (2009).
16. R. Kalluri, R. A. Weinberg, The Basics of Epithelial-Mesenchymal Transition. J. Clin. Investigation. 119(6), 1420-1428 (2009).
17. D. Susuki, S. Kimura, S. Naganuma, K. Tsuchiyama, T. Tanaka, N. Kitamura, S. Fujieda, H. Itoh, Regulation of microRNA Expression by Hepatocyte Growth Factor in Human Head and Neck Squamous Cell Carcinoma. Cancer Sci. 102(12), 2164-2171.
18. K. K. Ang, B. A. Berkey, X. Tu, H-Z. Zhang, R. Katz, E. H. Hammond, K. K. Fu, L. Milas, Impact of Epidermal Growth Factor Receptor Expression on Survival and Pattern of Relapse in Patients with Advanced Head and Neck Carcinoma. Cancer Res. 62, 7350-7356 (2002).
19. B. Kumar, K. G. Cordell, J. S. Lee, F. P. Worden, M. E. Price, H. H. Tran, G. T. Wolf, S. G. Urba, D. B. Chepeha, T. N. Teknos, A. Eisbruch, C. I. Tsien, J. M. G. Taylor, N. J. D'Silva, K. Yang, D. M. Kurnit, J. A. Bauer, C. R. Bradford, T. E. Carey, EGFR, p16, HPV Titer, Bc1-xL and p53, Sex, and Smoking as Indicators of Response to Therapy and Survival in Oropharyngeal Cancer. J. Clin. Oncology. 26, 3128-3137 (2008).
20. R. J. C. Slebos, Y. Yi, K. Ely, J. Carter, A. Evjen, X. Zhang, Y. Shyr, B. M. Murphy, A. J. Cmelak, B. B. Burkey, J. L. Netterville, S. Levy, W. G. Yarbrough, C. H. Chung, Gene Expression Differences Associated with Human Pappilomavirus Status in Head and Neck Squamous Cell Carcinoma. Clin. Cancer Res. 12(3), 701-709 (2006).
21. G. Muzio, M. Maggiora, E. Paiuzzi, R. A. Canuto, Aldehyde Dehydrogenases and Cell Proliferation. Free Radical Bio. Med. 52, 735-746 (2012).
22. A. Spira, J. Beane, V. Shah, G. Liu, F. Schembri, X. Yang, F. Palma, J. S. Brody, Effects of Cigarette Smoke on the Human Airway Epithelial Cell Transcriptome. Proc. Nat. Acad. Sci. 101(27), 10143-10148 (2004).
23. N. R. Hackett, A. Heguy, B-G. Harvey, T. P. O'Connor, K. Luettich, D. B. Flieder, R. Kaplan, R. G. Crystal, Variability of Antioxidant-Related Gene Expression in the Airway Epithelium of Cigarette Smokers. Amer. J. Respir. Cell and Mol. Bio. 29, 331-343.
24. M. Ji, H. Guan, C. Gao, B. Shi, P. Hou, Highly Frequent Promoter Methylation and PIK3CA Amplification in Non-Small Cell Lung Cancer (NSCLC). BMC Cancer. 11,147 (2011).
25. O. Kawano, H. Sasaki, K. Okuda, H. Yukiue, T. Yokoyama, M. Yano, Y. Fujii, PIK3CA Gene Amplification in Japanese Non-Small Cell Lung Cancer. Lung Cancer. 58, 159-160 (2007).
26. I. Imoto, Z-Q. Yang, A. Pimkhaokham, H. Tsuda, Y. Shimada, M. Imamura, M. Ohki, J. Inazawa, Identification of cIAP1 as a Candidate Target Gene within an Amplicon at 11q22 in Esophageal Squamous Cell Carcinoma. Cancer Res. 61, 6629-6634 (2001).
27. A. M. Lena, R. Shalom-Feuerstein, P. R. di Val Cervo, D. Aberdam, R. A. Knight, G. Melino, E. Candi, miR-203 Represses ‘Sternness” by Repressing ΔNp63. Cell Death Differentiation. 15, 1187-1195 (2008).
28. A. J. Bass, H. Watanabe, C. H. Mermel, S. Yu, S. Perner, R. G. Verhaak, S Y Kim, L. Wardwell, P. Tamayo, I. Gat-Viks, A. H. Ramos, M. S. Woo, B. A. Weir, G. Getz, R. Beroukhim, M. O'Kelly, A. Dutt, O. Rozenblatt-Rosen, P. Dziunycz, J. Komisarof, L. R. Chirieac, C. J. LaFargue, V. Scheble, T. Wilbertz, C. Ma, S. Rao, H. Nakagawa, D. B. Stairs, L. Lin, T. J. Giordano, P. Wagner, J. D. Minna, A. F. Gazdar, C. Q. Zhu, M. S. Brose, I. Cecconello, U. Ribeiro Jr., S. K. Marie, O. Dahl, R. A. Shivdasani, M-S. Tsao, M. A. Rubin, K. K. Wong, A. Regev, W. C. Hahn, D. G. Beer, A. K. Rustgi, M. Meyerson, SOX2 is an Amplified Lineage-Survival Oncogene in Lung and Esophageal Squamous Cell Carcinoma. Nature Genetics. 41(11), 1238-1242.
29. K. Okami, A. L. Reed, P. Cairns, W. M. Koch, W. H. Westra, S. Wehage, J. Jen, D. Sidransky, Cyclin D1 Amplification is Independent of p16 Inactivation in Head and Neck Squamous Cell Carcinoma. Oncogene. 18, 3541-3545 (1999).
30. A. Namazie, S. Alavi, O. I. Olopade, G. Pauletti, N. Aghamohammadi, M. Aghamohammadi, J. A. Gornbein, T. C. Calcaterra, D. J. Slamon, M. B. Wang, E. S. Srivatsan, Cyclin D1 Amplification and p16(MTS1/CDK4I) Deletion Correlate with Poor Prognosis in Head and Neck Tumors. Laryngoscope. 112, 472-481 (2002).
31. M. Fujii, R. Ishiguro, T. Yamashita, M. Tashiro, Cyclin D1 Amplification Correlates with Early Recurrence of Squamous Cell Carcinoma of the Tongue. Cancer Let. 172, 187-192 (2001).
32. Barretina, J., Caponigro, G., Stransky, N., Venkatsan, K., Margolin, A. A., Kim, S, Wilson, C. J., Lehar, J., Kryukov, G. V., Sonkin, D, Reddy, A., Liu, M., Murray, L., Berger, M. F., Monahan, J. E., Morais, P., Meltzer, J., Korejwa, A., Jane-Valbuena, J., Mapa, F. A., Thibault, J., Bric-Furlong, E., Raman, P., Shipway, A., Engels, I. H., Cheng, J., Yu, G. K., Yu, J., Aspesi, P. Jr, de Silva, M., Jagtap, K., Jones, M. D., Wang, L., Hatton, C., Palescandolo, E., Gupta, S., Mahan, S., Sougnez, C., Onofrio, R. C., Liefeld, T., MacConaill, L., Winckler, W., Reich, M., Li, N., Mesirov, J. P., Gabriel, S. B., Getz, G., Ardlie, K., Chan, V., Myer, V. E., Weber, B. L., Porter, J., Warmuth, M., Finan, P., Harris, J. L., Meyerson, M., Golub, T. R., Morrissey, M. P., Sellers, W. R., Schlegel, R., Garraway, L. A., The Cancer Cell Line Encyclopedia Enables Predictive Modelling of Anticancer Drug Sensitivity. Nature. 483 603-607 (2012).
33. X. Yang, H. Lu, B. Yan, R-A. Romano, Y. Bian, J. Friedman, P. Duggal, C. Allen, R. Chuang, R. Ehsanian, H. Si, S. Sinha, C. Van Waes, Z. Chen, ΔNp63 Versatilely Regulates a Broad NF-κB Gene Program and Promotes Squamous Epithelial Proliferation, Migration, and Inflammation. Cancer Res. 71, 3688-3700 (2011).
34. A. Chatterjee, X. Chang, T. Sen, R. Ravi, A. Bedi, D. Sidransky, Regulation of p53 Member Isoform ΔNp63a by the Nuclear Factor-κB Targeting Kinase IκB Kinase β. Cancer Res. 70, 1419-1429 (2010).
35. C. E. Barbieri, L. J. Tang, K. A. Brown, J. A. Pietenpol, Loss of p63 Leads to Increased Cell Migration and Up-Regulation of Genes Involved in Invasion and Metastatis. Cancer Res. 66, 7589-7597 (2006).
36. A. Martin, A. Cano, Tumorigensis: Twistl Links EMT to Self-Renewal. Nature Cell Bio. 12(10), 924-925 (2010).
37. T. Hussenet, S. Dali, J. Exinger, B. Monga, B. Jost, D. Dembele, N. Marinet, C. Thibault, J. Huelsken, E. Brambrilla, S. du Manoir, SOX2 is an Oncogene Activated by Recurrent 3q26.3 Amplifications in Human Lung Sqamous Cell Carcinomas. PLoS One. 5(1), e8960 (2010).
38. C. Chen, B. Koberle, A. M. Kaufmann, A. E. Albers, A Quest for Initiating Cells of Head and Neck Cancer and Their Treatment. Cancers. 2, 1528-1554 (2010).
39. J. M. G. Pedrero, D. G. Carracedo, C. M, Pinto, A. H. Zapatero, J. P. Rodrigo, C. S. Nieto, M. V. Gonzalez, Frequent Genetic and Biochemical Alterations of the PI 3-K/AKT/PTEN Pathway in Head and Neck Squamous Cell Carcinoma. Int. J. Cancer. 114, 242-248 (2005).
40. K. A. West, J. Brognard, A. S. Clark, I. R. Linnoila, X. Yang, S. M. Swain, C. Harris, S. Belinsky, P. A. Dennis, Rapid Akt Activation by Nicotine and A Tobacco Carcinogen Modulates the Phenotype of Normal Human Airway Epithelial Cells. J. Clin. Investigation. 111(1), 81-90 (2003).
41. M. E. Ritchie, J. Silver, A. Olshack, M. Holmes, D. Diyagama, A. Holloway, G. K. Smyth, A Comparison of Background Correction Methods for Two-Colour Microarrays. Bioinformatics. 23(20), 2700-2707 (2007).
42. V. G. Tusher, R. Tibshirani, G. Chu, Significance Analysis of Microarrays Applied to Transcriptional Responses to Ionizing Radiation. Proc. Nat. Acad. Sci. 98, 5116-5121 (2001).
43. M. Ashburner, C. A. Ball, J. A. Blake, D. Botstein, H. Butler, J. M. Cherry, A. P. Davis, K. Dolinski, S. S. Dwight, J. T. Eppig, M. A. Harris, D. P. Hill, L. Issel-Tarver, A. Kasarskis, S. Lewis, J. C. Matese, J. E. Richardson, M. Ringwald, G. M. Rubin, G. Sherlock, Gene Ontology: Tool for the Unification of Biology. Nature Genetics. 25, 25-29 (2000).
44. P. J. Rousseeuw, Silhouettes: A Graphical Aid to the Interpretation and Validation of Cluster Analysis. J. Comp. Appl. Math. 20, 53-65 (1987).
45. A. R. Dabney, ClaNC: Point-and-Click Software for Classifying Microarrays to Nearest Centroids. Bioinformatics. 22(1), 122-123 (2006).
46. H. Bengtsson, P. Wirapati, T. P. Speed, A Single-Array Preprocessing Method for Estimating Full-Resolution Raw Copy Numbers from All Affymetrix Genotyping Arrays Including GenomeWideSNP 5 & 6. Bioinformatics. 25(17) 2149-2156 (2009).
47. E. S. Venkatraman, A. B. Olshen, A Faster Circular Binary Segmentation Algorithm for the Analysis of Array CGH Data. Bioinformatics. 23(6), 657-663 (2007).
48. V. Walter, A. B. Nobel, F. A. Wright, DiNAMIC: A Method to Identify Recurrent DNA Copy Number Aberrations in Tumors. Bioinformatics. 27(5), 678-685 (2011).
49. M. D. Wilkerson, X. Yin, V. Walter, N. Zhao, C. R. Cabanski, M. C. Hayward, C. R. Miller, M. A. Socinski, A. M. Parsons, L. B. Thorne, B. E. Haithcock, N. K. Veeramachaneni, W. K. Funkhouser, S. H. Randell, P. S. Bernard, C. M. Perou, D. N. Hayes, Differential Pathogenesis of Lung Adenocarcinoma Subtypes Involving Sequence Mutations, Copy Number, Chromosomal Instability, and Methylation, PLoS ONE. 7 (5) e36530.
50. V. Walter, M. D. Wilkerson, D. N. Hayes, A. B. Nobel, F. A. Wright, unpublished material.

It is to be understood that, while the invention has been described in conjunction with the detailed description, thereof, the foregoing description is intended to illustrate and not limit the scope of the invention. Other aspects, advantages, and modifications of the invention are within the scope of the claims set forth below. All publications, patents, and patent applications cited in this specification are herein incorporated by reference as if each individual publication or patent application were specifically and individually indicated to be incorporated by reference.

1. A method for determining a prognosis for a patient with head and neck cancer which comprises:
(a) obtaining a suitable patient sample;
(b) measuring a nuclear p16 expression level; and
(c) comparing the nuclear p16 expression level from the patient sample with an expression level for a control sample, wherein the nuclear p16 expression level is indicative of the prognosis for the patient with head and neck cancer.

2. The method of claim 1, wherein the nuclear p16 expression level is reduced and the reduction is due to mutations or copy number loss.

3. The method of claim 1, which further comprises measuring levels of RB1 and p53 and a reduced level of RB1 or p53 in combination with a reduced nuclear p16 expression level indicates a poor prognosis.

4. The method of claim 1, which further comprises measuring levels of CCND1 wherein increased levels of CCND1 are indicative of a poor prognosis.

5. The method of claim 1, which further comprises measuring levels of expression associated with the atypical subtype wherein expression of the atypical subtype is indicative of a poor prognosis.

6. The method of claim 1, which further comprises measuring a cytoplasmic p16 expression level, wherein if the nuclear p16 expression level is reduced and the cytoplasmic p16 level is elevated in indicative of a particularly poor prognosis.

7. The method of claim 1, wherein the nuclear p16 expression levels are measured by an mRNA assay.

8. The method of claim 1, wherein the nuclear p16 expression levels are measured by a protein assay.

9. The method of claim 8, wherein the nuclear p16 expression levels are measured using antibodies.

10. The method of claim 1, wherein the patient sample is a biopsy sample.

11. The method of claim 10, wherein the biopsy sample is a lymph node biopsy sample.

12. The method of claim 1, wherein the head and neck cancer is a squamous cell carcinoma (SCC).

13. The method of claim 1, wherein the head and neck cancer is a hypopharynx, a glottis larynx, a larynx, a lip, a nasopharynx, an oral cavity, a salivary gland, a sinus, or a superglottic larynx cancer.

14. A method for determining a prognosis for a patient with head and neck cancer which comprises:
(a) obtaining a suitable patient sample;
(b) measuring a level of CCND1; and
(c) comparing the level of CCND1 from the patient sample with a level of CCND1 for a control sample, wherein the level of CCND1 is indicative of the prognosis for the patient with head and neck cancer.

15. A method for determining a prognosis for a patient with a solid tumor which comprises:
(a) obtaining a suitable patient sample;
(b) measuring p16 and RB1 genotypes, a CCND1 copy number, and a p16 nuclear protein expression level; and
(c) comparing the p16 and RB1 genotypes, the CCND1 copy number, and the p16 nuclear protein expression level from the patient sample with p16 and RB1 genotypes, a CCND1 copy number, and a p16 nuclear protein expression level associated with a control sample, wherein the p16 and RB1 genotypes, the CCND1 copy number, and the p16 nuclear protein expression level are indicative of the prognosis for the patient with the solid tumor.

16. The method of claim 13, further comprising measuring the expression of genes associated with an atypical subtype.

17. The method of claim 13, wherein the solid tumor is a solid tumor of epithelial origin.

18. The method of claim 13, wherein the solid tumor is a squamous cell carcinoma or a melanoma.

19-24. (canceled)
